(19) United States
(12) Patent Application Publication
(10) Pub. No.: US 2024/0218466 A1
(43) Pub. Date: Jul. 4, 2024

(54) DEVICES AND METHODS FOR DETECTION OF TARGET VIRUSES

(71) Applicant: Visby Medical, Inc., San Jose, CA (US)

(72) Inventors: Mackenzie HUNT, Soquel, CA (US); Karl GUEGLER, Menlo Park, CA (US); David SWENSON, Santa Clara, CA (US); Shaunak ROY, Sunnyvale, CA (US); Paul DENTINGER, Sunol, CA (US); Brian CIOPYK, Santa Clara, CA (US); Samira DASWANI, Redwood City, CA (US); Gary SCHOOLNIK, Palo Alto, CA (US); Teresa ABRAHAM, Washington, DC (US)

(21) Appl. No.: 17/907,095

(22) PCT Filed: Mar. 23, 2021
(86) PCT No.: PCT/US2021/023781

§ 371 Date: Sep. 22, 2022

Related U.S. Application Data
(60) Provisional application No. 62/993,637, filed on Mar. 23, 2020.

(51) Int. Cl. C12Q 1/70; C12Q 1/6818; C12Q 1/6876

(52) U.S. Cl. CPC C12Q 1/701; C12Q 1/6818; C12Q 1/6876; C12Q 2600/16

(57) ABSTRACT

A stand-alone molecular diagnostic test device includes a housing, a reverse transcription module, an amplification module, and a detection module. The reverse transcription module is configured to heat a biological sample to produce a target cDNA molecule associated with an RNA virus thereby producing an amplification solution. The amplification module defines a reaction volume configured to receive the amplification solution and amplify the target cDNA molecule within the amplification solution to produce an output. The detection module is configured to receive the output from the amplification module and includes one or more probes specific to a polynucleotide sequence of the RNA virus. The one or more probes is designed to facilitate production of a signal indicating the presence of the RNA virus in the biological sample.

### CROSS-REFERENCE TO RELATED APPLICATIONS

This application is a U.S. National Phase application, filed under 35 U.S.C. § 371(c), of International Application No. PCT/US2021/023781, filed Mar. 23, 2021, which claims benefit of priority to U.S. Provisional Application No. 62/993,637, filed Mar. 23, 2020, the entire contents of both of which are incorporated herein by reference.

### REFERENCE TO AN ELECTRONIC SEQUENCE LISTING

The contents of the electronic sequence listing (VISB-017_01US_sequencelisting_ST25; Size: 2,376 bytes; and Date of Creation: Apr. 26, 2021) is herein incorporated by reference in its entirety.

### BACKGROUND

The embodiments described herein relate to devices and methods for molecular diagnostic testing. More particularly, the embodiments described herein relate to disposable, self-contained devices and methods for molecular diagnostic testing that include reverse transcription capabilities. The embodiments described herein can be used to determine the presence of viruses, including influenza and SARS-COV-2.

There are over one billion infections in the U.S. each year, many of which are treated incorrectly due to inaccurate or delayed diagnostic results. Many known point of care (POC) tests have poor sensitivity (30-70%), while the more highly sensitive tests, such as those involving the specific detection of nucleic acids or molecular testing associated with a pathogenic target, are only available in laboratories. Thus, molecular diagnostics testing is often practiced in centralized laboratories. Known devices and methods for conducting laboratory-based molecular diagnostics testing, however, require trained personnel, regulated infrastructure, and expensive, high throughput instrumentation. Known high throughput laboratory equipment generally processes many (96 to 384 and more) samples at a time, therefore central lab testing is often done in batches. Known methods for processing test samples typically include processing all samples collected during a time period (e.g., a day) in one large run, resulting in a turn-around time of many hours to days after the sample is collected. Moreover, such known instrumentation and methods are designed to perform certain operations under the guidance of a skilled technician who adds reagents, oversees processing, and moves sample from step to step. Thus, although known laboratory tests and methods are very accurate, they often take considerable time, and are very expensive.

Although some known laboratory-based molecular diagnostics test methods and equipment offer flexibility (e.g., the ability to test for multiple different indications), such methods and equipment are not easily adaptable for point of care (“POC”) use or in-home use by an untrained user. Specifically, such known devices and methods are complicated to use and include expensive and sophisticated components. Thus, the use of such known laboratory-based methods and devices in a decentralized setting (e.g., POC or in-home use) would likely result in an increase in misuse, leading to inaccurate results or safety concerns. For example, many known laboratory-based systems include sophisticated optics and laser light sources, which can present a safety hazard to an untrained user. Some known systems can also require the user to handle or be exposed to reagents, which can be a safety risk for an untrained user. For example, some known systems use relatively large amounts of reagents and/or require replenishment of the reagents (e.g., within an instrument). In addition to being unsuitable for decentralized use, these known systems are also not suitable for long-term storage and shipping. Long-term storage can be desirable, for example to allow for stockpiling of assays for military applications, as a part of the CDC strategic national stockpile program, or other emergency preparedness initiatives.

Moreover, because of the flexibility offered by many known laboratory-based systems, such systems do not include lock-outs or mechanisms that prevent an untrained user from completing certain actions out of the proper sequence. For example, many known systems and methods include several distinct sample preparation operations, such as filtering, washing, lysing, and addition of sample preparation reagents to preserve the target nucleic acids. If such operations are not performed in a predetermined order and/or within predetermined time limits, the accuracy of the test can be compromised. Some known systems attempt to limit the complexities associated with sample preparation by limiting the analysis to only “clean” samples. As a result, such systems do not enable true end-to-end molecular diagnostic methods, because the detailed sample preparation must still be performed by an upstream process.

Although recent advances in technology have enabled the development of “lab on a chip” devices, such devices are often not optimized for point-of-care testing or in-home use. For example, some known devices and methods require an expensive or complicated instrument to interface with the test cartridge, thus increasing the likelihood of misuse. Additionally, many known “lab on a chip” devices amplify a very small volume of sample (e.g., less than one microliter), and are therefore not suited for analyzing for multiple different indications (e.g., a 3-plex or 4-plex test). Moreover, devices that produce such small sample volumes often include optical detection using photocells, charge coupled devices (CCD cameras) or the like, because the sample volumes are too small to produce an output that can be read by the naked eye or less sophisticated (and costly) detectors.

Some known molecular diagnostic systems and methods facilitate detection of viral pathogens by performing reverse transcription polymerase chain reaction (RT-PCR). Although such methods are useful isolating and detecting viruses, they can be complex, thus rendering many know systems and methods unsuitable for decentralized and/or point-of-care use. For example, some known RT-PCR methods include additional steps to isolate and protect the target RNA from rapid degradation from ribonuclease (RNase). Inconsistencies when performing such methods can lead to inaccurate results due to variation in the RNA degradation. Thus, known RT-PCR devices and methods not suitable for use by untrained users.

In addition to the need for decentralized testing (e.g., testing at a point-of-care, a patient's home, work sites, or other public venues), there has been increased interest in combining or “pooling” samples to allow multiple users (or specimens) to be tested with one diagnostic test. This approach increases the number of individuals that can be tested using the same amount of resources. For example, multiple individuals can be tested together, using only the resources and time needed for a single test. In this manner, groups of individuals can be tested more quickly to facilitate rapid screening (e.g., for entry or participation in public events, to ensure workplace safety, to facilitate safe travel, etc.). In such pooled sample testing, if the test produces a negative result, then all of the individuals (or specimens) are considered as negative. If, however, the test produces a positive result, then each individual sample can be tested individually to determine which individual or specimen is positive.

Because the samples are diluted, however, there is a greater likelihood of false negatives, thereby potentially reducing the sensitivity of the diagnostic test. Moreover, some forms of sample pooling include placing multiple swab samples into a standard amount of transport media. This can produce a higher concentration of constituents (e.g., mucin, swab materials, and the like) that can increase inhibition of the downstream molecular diagnostic test. Thus, many known molecular diagnostic systems and methods are not suitable for pooled sample testing.

Thus, a need exists for improved devices and methods for molecular diagnostic testing. In particular, a need exists for improved devices and methods that are suitable for long-term storage. A need also exists for improved devices and methods that are easy to use and that can be performed with minimal user input. A need also exists for improved devices and methods that can receive a wide range of samples (e.g., raw samples, such as throat swabs, sputum, nasal swabs, urine, saliva, and blood). A need also exists for improved devices and methods that include a reverse transcription module or that otherwise allows for detection of a target RNA. A need also exists for improved devices, kits and methods that are suitable for testing pooled samples.

### SUMMARY

Molecular diagnostic test devices for amplifying a nucleic acid within a sample and producing an indicator of a target molecule (e.g., DNA or RNA) in the sample are described herein. In some embodiments, a stand-alone molecular diagnostic test device includes a housing, a reverse transcription module, an amplification module, and a detection module. The reverse transcription module is configured to heat a biological sample to produce a target cDNA molecule associated with an RNA virus thereby producing an amplification solution. The amplification module defines a reaction volume configured to receive the amplification solution and amplify the target cDNA molecule within the amplification solution to produce an output. The detection module is configured to receive the output from the amplification module and includes one or more probes specific to a polynucleotide sequence of the RNA virus. The one or more probes is designed to facilitate production of a signal indicating the presence of the RNA virus in the biological sample.

In some embodiments, a method of detecting a target pathogen in multiple biological samples using a stand-alone molecular diagnostic test device that includes a sample preparation module, an amplification module, and a detection module integrated therein. The method includes combining each of the biological samples to form a combined input sample. The combined input sample is conveyed into the stand-alone molecular diagnostic test device via an input opening. The stand-alone molecular diagnostic test device is actuated to cause the stand-alone molecular diagnostic test device to heat the combined input sample within the sample preparation module to produce a complementary DNA (cDNA) associated with the target pathogen. The device further heats the combined input sample within the amplification module to amplify the cDNA within the combined input sample to produce an output solution containing a target amplicon. The output solution and a reagent formulated to produce a signal that indicates a presence of the target amplicon within the output solution are reacted within the detection module. The detection module including one or more probes specific to a polynucleotide sequence of the target pathogen.

In some embodiments, the target pathogen is a virus and each of the biological samples is from a swab. In some embodiments, each of the biological samples is collected from one of a nasal swab, a mid-turbinate swab, a nasopharyngeal swab, or an oropharyngeal swab.

In some embodiments, the number of biological sample includes between two biological samples and twenty biological samples. In some embodiments, the number of biological sample includes between three biological samples and ten biological sample. In some embodiments, the number of biological sample includes between four biological samples and eight biological samples.

In some embodiments, the multiple biological samples are combined by conveying each of the biological samples into a sample tube containing transport media and/or a buffer. In some embodiments, the multiple biological samples are combined by conveying each of the biological samples into a corresponding sample tube containing transport media and/or a buffer and mixing the contents of each of the sample tubes.

In some embodiments, at least one of the sample tube or the sample preparation module includes a reducing agent. The reducing agent can be any of lithium, sodium, potassium, aluminum amalgam, lithium aluminum hydride, sodium borohydride, sodium cyanoborohydride, lithium tri-butoxy aluminum hydride, dibutoxy aluminum hydride, lithium triethyl borohydride, tris(2-carboxyethyl)phosphine (TCEP), dithiothreitol (DTT), mercaptoethanol, cysteine, thioglycerol, thioglycolic acid, cysteamine, glutathione, N-acetylcysteine (NAC), β-mercaptoethanol, sodium dodecyl sulfate (SDS), sodium sulfite, ammonium sulfamate, sodium bisulfite, dithionite metabisulfite sulfur dioxide, vitamin C, ascorbic acid, and dimethylsulfoxide (DMSO). In some embodiments, the reducing agent is N-acetylcysteine (NAC), e.g. N-acetyl-L-cysteine. In some embodiments, the reducing agent is a pellet stored within the sample preparation module. The pellet can include any of enzymes, nucleic acids, or a buffer composition, and optionally a surfactant.

### BRIEF DESCRIPTION OF THE DRAWINGS

FIG. 1 is a diagram illustrating an enzyme linked reaction, according to an embodiment, resulting in the production a signal.

FIG. 2 is a schematic illustration of a molecular diagnostic test device, according to an embodiment.

FIGS. 3 and 4 are a perspective view and a top view, respectively, of a molecular diagnostic test device, according to an embodiment.

FIG. 5 is a perspective view of the molecular diagnostic test device shown in FIGS. 3 and 4, with the lid removed to show the sample input opening.

FIG. 6 is a perspective view of the molecular diagnostic test device shown in FIGS. 3 and 4, with the top portion of the housing removed to show the internal components.

FIG. 7 is an exploded view of the molecular diagnostic test device shown in FIGS. 3 and 4.

FIG. 8 is a top perspective exploded view of a portion of the housing assembly of the molecular diagnostic test device shown in FIGS. 3 and 4.

FIG. 9 is an enlarged view of a portion of the top housing shown in FIG. 8, showing lid engagement portion.

FIGS. 10 and 11 are a front perspective view and a rear perspective view, respectively, of the housing assembly of the molecular diagnostic test device shown in FIGS. 3 and 4.

FIG. 12 is a perspective exploded view of the housing assembly shown in FIGS. 10 and 11.

FIG. 13 is a perspective view of the vertical manifold of the housing assembly shown in FIGS. 10 and 11.

FIG. 14 is a perspective view of the sample transfer manifold of the housing assembly shown in FIGS. 10 and 11, including a filter assembly and an inactivation assembly coupled thereto.

FIGS. 15-18 are a front perspective view, a rear perspective view, a bottom view, and a top view, respectively, of the sample transfer manifold of the housing assembly shown in FIGS. 10 and 11.

FIGS. 19-22 are cross-sectional views of the sample transfer manifold shown in FIGS. 15-18 taken along line X₁-X₁, X₂-X₂, X₃-X₃, and X₄-X₄, respectively.

FIGS. 23-25 are a top perspective view, a bottom perspective view, and a side perspective view, respectively, of a sample input actuator of the molecular diagnostic test device shown in FIGS. 3 and 4.

FIGS. 26 and 27 are a top perspective view and a bottom perspective view, respectively, of a sample lid of the molecular diagnostic test device shown in FIGS. 3 and 4.

FIGS. 28-30 are a side perspective view, a bottom perspective view, and a rear perspective view, respectively, of a first reagent actuator of the molecular diagnostic test device shown in FIGS. 3 and 4.

FIGS. 31 and 32 are a rear perspective view and a bottom perspective view, respectively, of a second reagent actuator of the molecular diagnostic test device shown in FIGS. 3 and 4.

FIG. 33 is a perspective view of a spring clip that can be coupled to the sample input actuator, as shown in FIG. 25.

FIGS. 34 and 35 are a front perspective view and a rear perspective view, respectively, of a lock lever coupled to the sample transfer manifold shown in FIG. 16.

FIGS. 36 and 37 are a perspective view and a top view, respectively, of a filter assembly of the molecular diagnostic test device shown in FIGS. 3 and 4.

FIGS. 38 and 39 are cross-sectional views of the filter assembly shown in FIGS. 36 and 37 taken along line X-X in FIG. 37, with the filter assembly in a first configuration and a second configuration, respectively.

FIGS. 40-42 are a top perspective view, a bottom perspective view, and a bottom view, respectively, of a lysing module of the molecular diagnostic test device shown in FIGS. 3 and 4.

FIGS. 43 and 44 are cross-sectional views of the lysing module shown in FIGS. 40 and 41 taken along line X₁-X₁ and line X₂-X₂ in FIG. 42, respectively.

FIG. 45 is an enlarged view of a portion of the top housing of the molecular diagnostic test device shown in FIGS. 3 and 4, showing the lid in a closed position.

FIG. 46 is perspective cross-sectional view of a portion of the top housing of the molecular diagnostic test device shown in FIGS. 3 and 4, showing the lid in the closed position.

FIGS. 47 and 48 are a perspective cross-sectional view and a rear view, respectively, of a portion of the molecular diagnostic test device shown in FIGS. 3 and 4, showing the lid in the closed position and the sample input actuator in its first position.

FIGS. 49 and 50 are a perspective view and a rear view, respectively, of the molecular diagnostic test device shown in FIGS. 3 and 4, showing the lid in the closed position and the sample input actuator in its second position.

FIGS. 51 and 52 are cross-sectional views of a portion of the molecular diagnostic test device shown in FIGS. 3 and 4 with the sample input actuator in its second position taken along line X₁-X₁ in FIG. 48 and line X₂-X₂ in FIG. 50, respectively.

FIGS. 53 and 54 are a perspective view and a rear view, respectively, of the molecular diagnostic test device shown in FIGS. 3 and 4, showing the sample input actuator in its second position and the first reagent actuator in its second position.

FIGS. 55 and 56 are a perspective view and a side cross-sectional view, respectively, of the molecular diagnostic test device shown in FIGS. 3 and 4, showing the sample input actuator in its second position, the first reagent actuator in its second position, and the second reagent actuator in its second position.

FIGS. 57 and 58 are a perspective exploded view and a front view, respectively, of a detection module of the molecular diagnostic test device shown in FIGS. 3 and 4.

FIG. 59 is a schematic illustration of a molecular diagnostic test device, according to an embodiment.

FIGS. 60 and 61 are a perspective view and a top view, respectively, of a molecular diagnostic test device, according to an embodiment.

FIGS. 62 and 63 are exploded views of the molecular diagnostic test device shown in FIGS. 60 and 61.

FIGS. 64 and 65 are a front perspective view (FIG. 64) and a rear perspective view (FIG. 65) of the molecular diagnostic test device shown in FIGS. 60 and 61, with the housing removed to show the modules therein.

FIG. 66 is an exploded perspective view of the housing assembly of the molecular diagnostic test device shown in FIGS. 60 and 61.

FIG. 67 is a bottom perspective view of the top housing of the molecular diagnostic test device shown in FIGS. 60 and 61.

FIGS. 68-70 are a front perspective view (FIG. 68), a rear perspective view (FIG. 69), and a bottom perspective view (FIG. 70) of the lid of the molecular diagnostic test device shown in FIGS. 60 and 61.

FIGS. 71 and 72 are a top perspective view (FIG. 71) and a bottom perspective view (FIG. 72) of the flexible plate of the molecular diagnostic test device shown in FIGS. 60 and 61.

FIGS. 73 and 74 are side cross-sectional views taken along line X-X in FIG. 61, showing the molecular diagnostic test device in a first (pre-actuated) configuration and a second (post-actuated) configuration, respectively.

FIGS. 75 and 76 are a top perspective view (FIG. 75) and a bottom perspective view (FIG. 76) of the deformable support member of the molecular diagnostic test device shown in FIGS. 60 and 61.

FIGS. 77 and 78 are a perspective view (FIG. 77) and a top view (FIG. 78) of the sample preparation (or staging) module of the molecular diagnostic test device shown in FIGS. 60 and 61.

FIGS. 79 and 80 are a cross-sectional view (FIG. 79) and an exploded view (FIG. 80) of the sample preparation module shown in FIGS. 77 and 78.

FIG. 81 is a cross-sectional view taken along line X-X in FIG. 78 of the mixing assembly of the sample preparation module shown in FIGS. 77 and 78.

FIG. 82 is atop view of a flow member of the amplification module of the molecular diagnostic test device shown in FIGS. 60 and 61.

FIG. 83 is an exploded view of the detection module of the molecular diagnostic test device shown in FIGS. 60 and 61.

FIGS. 84 and 85 are a top perspective view (FIG. 84) and a bottom perspective view (FIG. 85) of the reagent module of the molecular diagnostic test device shown in FIGS. 60 and 61.

FIG. 86 is a front perspective view of rotary valve assembly of the molecular diagnostic test device shown in FIGS. 60 and 61.

FIGS. 87-92 are front views of the rotary valve assembly shown in FIG. 86 with the vent housing being “transparent” to show the valve disc in each of six different operational configurations.

FIGS. 93A-93C are perspective views of the molecular diagnostic device shown in FIGS. 60 and 61 in various stages of operation, according to an embodiment.

FIGS. 94 and 95 are a flow charts of a methods of detecting a target pathogen in a combined biological sample, each according to an embodiment.

FIG. 96 is a flow chart of a method of detecting a coronavirus, according to an embodiment.

FIG. 97 is a photograph showing results from Flu A/B tests conducted on several stand-alone molecular diagnostic test devices as described herein.

FIG. 98A shows output Cycle Threshold (Ct) values, assessed by amplification of input solution in the presence of NAC by quantitative PCR on a LightCycler. FIG. 98B shows the data of FIG. 98 plotted as a bar graph

FIG. 99A shows detection of coronavirus genomic RNA in a handheld device without the NAC reducing agent present in the buffer. FIG. 99B shows detection of coronavirus genomic RNA in a handheld device with NAC reducing agent present in the buffer.

FIG. 100A shows detection of coronavirus genomic RNA in a handheld device without NAC present in the device sample port. FIG. 100B shows detection of coronavirus genomic RNA in a handheld device with lyophilized NAC present in the device sample port at 2.5 mM when rehydrated with the sample. FIG. 100C shows detection of coronavirus genomic RNA in a handheld device with lyophilized NAC present in the device sample port at 5 mM when rehydrated with the sample.

### DETAILED DESCRIPTION

In some embodiments, an apparatus is configured for a disposable, portable, single-use, inexpensive, molecular diagnostic approach. The apparatus can include one or more modules configured to perform high quality molecular diagnostic tests, including, but not limited to, sample preparation, nucleic acid amplification (e.g., via polymerase chain reaction, isothermal amplification, or the like), and detection. In some embodiments, sample preparation can be performed by isolating the target pathogen/entity and removing unwanted amplification (e.g., PCR) inhibitors. The target entity can be subsequently lysed to release target nucleic acid for amplification. A target nucleic acid in the target entity can be amplified with a polymerase undergoing temperature cycling or via an isothermal incubation to yield a greater number of copies of the target nucleic acid sequence for detection.

In some embodiments, the devices described herein are stand-alone devices that include all necessary substances, mechanisms, and subassemblies to perform any of the molecular diagnostic tests described herein. Such stand-alone devices do not require any external instrument to manipulate the biological samples, and only require connection to a power source (e.g., a connection to an A/C power source, coupling to a battery, or the like) to complete the methods described herein. For example, the device described herein do not require any external instrument to heat the sample, agitate or mix the sample, to pump (or move) fluids within a flow member, or the like. Rather, the embodiments described herein are fully-contained and upon add a biological sample and being coupled to a power source, the device can be actuated to perform the molecular diagnostic tests described herein. Thus, the stand-alone molecular diagnostic test devices described herein can be described as “instrument-free”devices. In some embodiments, the method of actuating the device can be such that the device is a CLIA-waived device and/or can operate in accordance with methods that are CLIA waived.

In some embodiments, a method of detecting a target molecule includes “one-step” or “single button” actuation of a device. For example, in some embodiments, a method includes coupling the molecular diagnostic test device to a power source. A biological sample is conveyed into a sample preparation module within the molecular diagnostic test device via an input opening. The molecular diagnostic test device is then actuated by only a single action to cause the molecular diagnostic test device to perform the following functions without further user action. First, the device heats the biological sample via a heater of the sample preparation module to lyse a portion of the biological sample to produce an input sample. Second, the device conveys the input sample to an amplification module within the molecular diagnostic test device. The device then heats the input sample within a reaction volume of the amplification module to amplify the nucleic acid within the input sample thereby producing an output solution containing a target amplicon. The device then reacts, within a detection module of the molecular diagnostic test device, each of (i) the output solution and (ii) a reagent formulated to produce a signal that indicates a presence of the target amplicon within the output solution. The detection module includes a detection surface configured to capture the target amplicon to produce the signal. A result associated with the signal is then read.

In some embodiments, a method of detecting a target pathogen includes combining multiple samples to allow multiple individuals (or specimens) to be tested in a single test device and in a single test event. The embodiments described herein increases the number of individuals that can be tested using the same amount of resources and in the same time period that is routinely used for testing a single sample. In some embodiments, the method includes combining each of the biological samples to form a combined input sample. The combined input sample is conveyed into the stand-alone molecular diagnostic test device via an input opening. The stand-alone molecular diagnostic test device is actuated to cause the stand-alone molecular diagnostic test device to heat the combined input sample within the sample preparation module to produce a complementary DNA (cDNA) associated with the target pathogen. The device further heats the combined input sample within the amplification module to amplify the cDNA within the combined input sample to produce an output solution containing a target amplicon. The output solution and a reagent formulated to produce a signal that indicates a presence of the target amplicon within the output solution are reacted within the detection module. The detection module including one or more probes specific to a polynucleotide sequence of the target pathogen.

In some embodiments, the target pathogen is a virus and each of the biological samples is from a swab. In some embodiments, each of the biological samples is collected from one of a nasal swab, a mid-turbinate swab, a nasopharyngeal swab, or an oropharyngeal swab.

In some embodiments, the number of biological sample includes between two biological samples and twenty biological samples. In some embodiments, the number of biological sample includes between three biological samples and ten biological sample. In some embodiments, the number of biological sample includes between four biological samples and eight biological samples.

In some embodiments, the multiple biological samples are combined by conveying each of the biological samples into a sample tube containing transport media and/or a buffer. In some embodiments, the multiple biological samples are combined by conveying each of the biological samples into a corresponding sample tube containing transport media and/or a buffer and mixing the contents of each of the sample tubes.

In some embodiments, at least one of the sample tube or the sample preparation module includes a reducing agent. The reducing agent can be any of lithium, sodium, potassium, aluminum amalgam, lithium aluminum hydride, sodium borohydride, sodium cyanoborohydride, lithium tri-butoxy aluminum hydride, dibutoxy aluminum hydride, lithium triethyl borohydride, tris(2-carboxyethyl)phosphine (TCEP), dithiothreitol (DTT), mercaptoethanol, cysteine, thioglycerol, thioglycolic acid, cysteamine, glutathione, N-acetylcysteine (NAC), β-mercaptoethanol, sodium dodecyl sulfate (SDS), sodium sulfite, ammonium sulfamate, sodium bisulfite, dithionite metabisulfite sulfur dioxide, vitamin C, ascorbic acid, and dimethylsulfoxide (DMSO). In some embodiments, the reducing agent is N-acetylcysteine (NAC). In some embodiments, the reducing agent is a pellet stored within the sample preparation module. The pellet can include any of enzymes, nucleic acids, or a buffer composition, and optionally a surfactant.

In some embodiments, the signal is produced within about 30 minutes after the actuating the stand-alone molecular diagnostic test device.

In some embodiments, the method includes reading a result associated with the signal. For example, in some embodiments, the signal can be a visible signal (e.g., a color spot) produced within the detection module that is visible to the user via an external surface of the stand-alone molecular diagnostic test device. To read the result, the user can visually inspect for the presences of the signal. In other embodiments, the detection module need not be viewable via an external surface of the stand-alone molecular diagnostic test device. In such embodiments, the stand-alone molecular diagnostic test device can produce one or more electronic outputs (e.g., light signals, audible signals, haptic signals, wireless signals, etc.) and the result can be read by the user receiving such electronic outputs. In some embodiments, the stand-alone molecular diagnostic test device includes a radio (e.g., a Bluetooth® processor) configured to electronically communicate with a computing device via a short-range wireless communication protocol and the electronic output includes a wireless signal indicating the presence of the target pathogen.

In some embodiments, the method includes conducting a second test on a second stand-alone molecular diagnostic device when the result from the first test indicates the presence of the target pathogen. The second test can be conducted on one of the biological samples.

In some embodiments, a kit for detecting a target RNA virus in multiple biological samples that are combined (i.e., pooled) includes a sample tube, a sample transfer device, and a stand-alone molecular diagnostic test device. The sample tube contains a buffer and is configured to receive the biological samples to form a combined input sample. The stand-alone molecular diagnostic test device includes a housing, a sample preparation module, an amplification module, and a detection module. The sample preparation module, the amplification module, and the detection module are each within the housing. The housing define an input opening through which the combined input sample can be conveyed from the sample tube into the device using the sample transfer device. The sample preparation module is configured to heat the combined input sample to produce complementary DNA (cDNA) associated with the RNA virus thereby producing an amplification solution. The amplification module defines a reaction volume configured to receive the amplification solution and amplify the cDNA molecule within the amplification solution to produce an output. The detection module is configured to receive the output from the amplification module, and includes one or more probes specific to a polynucleotide sequence of the RNA virus. The one or more probes are designed to facilitate production of a signal indicating the presence of the RNA virus in the combined input sample.

In some embodiments, the kit further includes a reducing agent stored within one of the sample tube or the sample preparation module. The reducing agent can be any of lithium, sodium, potassium, aluminum amalgam, lithium aluminum hydride, sodium borohydride, sodium cyanoborohydride, lithium tri-butoxy aluminum hydride, dibutoxy aluminum hydride, lithium triethyl borohydride, tris(2-carboxyethyl)phosphine (TCEP), dithiothreitol (DTT), mercaptoethanol, cysteine, thioglycerol, thioglycolic acid, cysteamine, glutathione, N-acetylcysteine (NAC), β-mercaptoethanol, sodium dodecyl sulfate (SDS), sodium sulfite, ammonium sulfamate, sodium bisulfite, dithionite metabisulfite sulfur dioxide, vitamin C, ascorbic acid, and dimethylsulfoxide (DMSO). In some embodiments, the reducing agent is N-acetylcysteine (NAC). In some embodiments, the reducing agent is a pellet stored within the sample preparation module. The pellet can include any of enzymes, nucleic acids, or a buffer composition, and optionally a surfactant.

In some embodiments, a method of detecting a coronavirus from multiple biological samples using a stand-alone molecular diagnostic test device includes combining a biological sample from each of the patients to form a combined input sample. The input sample is conveyed to a reverse transcription module within a housing of the stand-alone molecular diagnostic test device, and then is heated therein to produce a target DNA molecule associated with the coronavirus. The input sample is conveyed from the reverse transcription module to an amplification module within the housing. The amplification module defines a reaction volume within which the input sample is heated to amplify the target DNA molecule within the input sample thereby producing an output solution. The method includes conveying into a detection module each of A) the output solution and B) a reagent formulated to produce a signal that indicates a presence of the coronavirus within the output solution. The detection module includes one or more probes specific to a coronavirus polynucleotide sequence.

In some embodiments, the number of patients is between two and twenty. In some embodiment, the number of patients is between three and ten. In some embodiments, the number of patients is between four and eight.

In some embodiments, a stand-alone molecular diagnostic test device includes a housing, a reverse transcription module, and amplification module, and a detection module. The sample preparation module, the amplification module, and the detection module are each within the housing. The reverse transcription module is configured to heat a biological sample to produce a target cDNA molecule associated with an RNA virus thereby producing an amplification solution. The amplification module defines a reaction volume configured to receive the amplification solution and amplify the target cDNA molecule within the amplification solution to produce an output. The detection module is configured to receive the output from the amplification module and includes one or more probes specific to a polynucleotide sequence of the RNA virus. The one or more probes are designed to facilitate production of a signal indicating the presence of the RNA virus in the biological sample.

In some embodiments, the RNA virus is influenza, optionally influenza A or influenza B. In some embodiments, the RNA virus is a paramyxovirus, optionally respiratory syncytial virus. In some embodiments, the RNA virus is a coronavirus, optionally a betacoronavirus. In some embodiments, the RNA virus is a coronavirus, optionally Covid-19.

In some embodiments, the one or more probes includes a first probe and a second probe. The first probe is specific to a first polynucleotide sequence of one of an influenza virus, a paramyxovirus, or a coronavirus and the second probe specific to a second polynucleotide sequence of another of the influenza virus, the paramyxovirus, or the coronavirus. The detection module includes a first detection surface to which the first probe is adhered and a second detection surface to which the second probe is adhered. A first signal is produced from the first detection surface when the first polynucleotide sequence is present and a second signal is produced from the second detection surface when the second polynucleotide sequence is present.

In some embodiments, the stand-alone molecular diagnostic test device includes a reagent module within the housing. The reagent module includes a reagent formulated produce a first color product from the first detection surface and a second color product from the second detection surface when the reagent is conveyed into the detection module. In some embodiments, the detection module is positioned within the housing such that the first color product and the second color product are viewable via a detection opening of the housing. In some embodiments, the stand-alone molecular diagnostic test device includes an electronic system including a digital read module implemented in at least one of a memory or a processing device. The digital read module is configured to: A) detect the presence of the first color product and the second color product and B) produce an electronic output associated with the presence of at least one the first color product and the second color product.

In some embodiments, a stand-alone molecular diagnostic test device includes a housing, a reverse transcription module, and amplification module, and a detection module. The sample preparation module, the amplification module, and the detection module are each within the housing. The reverse transcription module is configured to heat a biological sample to produce a target cDNA molecule associated with a coronavirus thereby producing an amplification solution. The amplification module defines a reaction volume configured to receive the amplification solution and amplify the target cDNA molecule within the amplification solution to produce an output. The detection module is configured to receive the output from the amplification module. The detection module includes one or more probes specific to a coronavirus polynucleotide sequence, which are designed to facilitate production of a signal indicating the presence of the coronavirus in the biological sample.

In some embodiments, a method of detecting a coronavirus using a disposable molecular diagnostic test device includes conveying a biological sample to a reverse transcription module within a housing of the disposable molecular diagnostic test device. The biological sample is heated within the reverse transcription module to produce a target DNA molecule associated with the coronavirus. The biological sample is conveyed from the reverse transcription module to an amplification module within the housing. The biological sample is heated within a reaction volume of the amplification module via a heater to amplify the target DNA molecule within the input sample thereby producing an output solution. Each of the output solution and a reagent formulated to produce a signal that indicates a presence of the coronavirus within the output solution are conveyed into the detection module. The detection module includes one or more probes specific to a coronavirus polynucleotide sequence. The one or more probes are adhered to a detection surface within the detection module.

In some embodiments, a probe set for detecting the presence of at least any of an influenza virus, a paramyxovirus, or a coronavirus comprises a first probe and a second probe. The first probe is specific to a first polynucleotide sequence of one of the influenza virus, the paramyxovirus, or the coronavirus. The second probe is specific to a second polynucleotide sequence of another of the influenza virus, the paramyxovirus, or the coronavirus. Each of the first probe and the second probe has a melting temperature within about 5 degrees C. of a reference melting temperature.

In some embodiments, the first probe is specific to the first polynucleotide sequence of the influenza virus and the second probe is specific to the second polynucleotide sequence of the paramyxovirus. The probe set further comprises a third probe specific to a third polynucleotide sequence of the coronavirus.

As used in this specification and the appended claims, the term “reagent” includes any substance that is used in connection with any of the reactions described herein. For example, a reagent can include an elution buffer, a PCR reagent, an enzyme, a substrate, a wash solution, a blocking solution, or the like. A reagent can include a mixture of one or more constituents. A reagent can include such constituents regardless of their state of matter (e.g., solid, liquid or gas). Moreover, a reagent can include the multiple constituents that can be included in a substance in a mixed state, in an unmixed state and/or in a partially mixed state. A reagent can include both active constituents and inert constituents. Accordingly, as used herein, a reagent can include non-active and/or inert constituents such as, water, colorant or the like.

The term “nucleic acid molecule,” “nucleic acid,” or “polynucleotide” may be used interchangeably herein, and may refer to deoxyribonucleic acid (DNA) or ribonucleic acid (RNA), including known analogs or a combination thereof unless otherwise indicated. Nucleic acid molecules to be profiled herein can be obtained from any source of nucleic acid. The nucleic acid molecule can be single-stranded or double-stranded. In some cases, the nucleic acid molecules are DNA The DNA can be mitochondrial DNA, complementary DNA (cDNA), or genomic DNA. In some cases, the nucleic acid molecules are genomic DNA (gDNA). The DNA can be plasmid DNA, cosmid DNA, bacterial artificial chromosome (BAC), or yeast artificial chromosome (YAC). The DNA can be derived from one or more chromosomes. For example, if the DNA is from a human, the DNA can be derived from one or more of chromosomes 1, 2, 3, 4, 5, 6, 7, 8, 9, 10, 11, 12, 13, 14, 15, 16, 17, 18, 19, 20, 21, 22, X, or Y. In some cases, the nucleic acid molecules are RNA can include, but is not limited to, mRNAs, tRNAs, snRNAs, rRNAs, retroviruses, small non-coding RNAs, microRNAs, polysomal RNAs, pre-mRNAs, intronic RNA, viral RNA, cell free RNA and fragments thereof. The non-coding RNA, or ncRNA can include snoRNAs, microRNAs, siRNAs, piRNAs and long nc RNAs. The source of nucleic acid for use in the devices, methods, and compositions described herein can be a sample comprising the nucleic acid.

Unless indicated otherwise, the terms apparatus, diagnostic apparatus, diagnostic system, diagnostic test, diagnostic test system, test unit, and variants thereof, can be interchangeably used.

The methods described herein can be performed on any suitable molecular diagnostic device, such as any of the diagnostic devices shown and described herein or in International Patent Publication No. WO2016/109691, entitled “Devices and Methods for Molecular Diagnostic Testing,” International Patent Publication No. WO2017/185067, entitled “Printed Circuit Board Heater for an Amplification Module,” International Patent Publication No. WO2018/005710, entitled “Devices and Methods for Detection of Molecules Using a Flow Cell,” International Patent Publication No. WO2018/005870, entitled “Devices and Methods for Nucleic Acid Extraction,” and U.S. Patent Publication No. 2019/0169677, entitled “Portable Molecular Diagnostic Device and Methods for the Detection of Target Viruses,” each of which is incorporated herein by reference in its entirety.

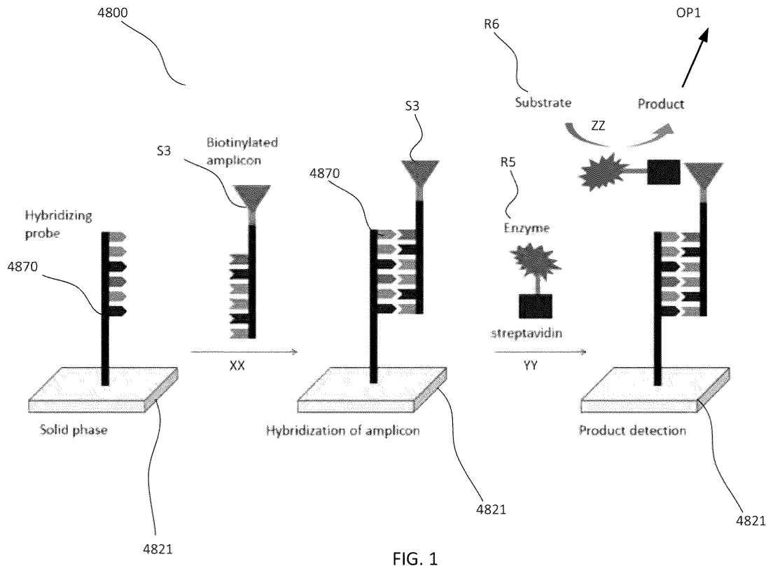

FIG. 1 illustrates a portion of the operations and/or features associated with an enzymatic reaction, according to an embodiment, that can be conducted by or within the detection the detection module 4800, or any other detection module described herein. In some embodiments, the enzymatic reaction can be carried out to facilitate visual detection of a molecular diagnostic test result using any devices or systems described herein. In other embodiments, the enzymatic reaction need not be performed to produce visual detection. For example, as described herein, in some embodiments, the methods that employ the illustrated enzymatic reaction can employ alternative methods to read a result associated with signal produced.

In some embodiments, the reaction, the detection module 4800, and/or the remaining components within the device 4000 (or any devices described herein) can be collectively configured such that the device is a single-use device that can be used in a point-of-care setting and/or in a user's home. Similarly stated, in some embodiments, the device 4000 (and any of the other devices shown and described herein) can be configured for use in a decentralized test facility. Further, in some embodiments, the reaction shown in FIG. 1 can facilitate the device 4000 (and any of the other devices shown and described herein) operating with sufficient simplicity and accuracy to be a CLIA-waived device. Similarly stated, in some embodiments, the reaction shown in FIG. 1 can provide the output signal OP1 in a manner that poses a limited likelihood of misuse and/or that poses a limited risk of harm if used improperly. In some embodiments, the reaction can be successfully completed within the device 4000 (or any other device described herein) upon actuation by a user with minimal (or no) scientific training, in accordance with methods that require little judgment of the user, and/or in which certain operational steps are easily and/or automatically controlled.

As shown, the detection module 4800 includes a detection surface 4821 within a read lane or flow channel. The detection surface 4821 is spotted and/or covalently bonded with a specific hybridizing probe 4870, such as an oligonucleotide. The hybridizing probe 4870 (also referred to as a capture probe) can be similar to any of the capture probes described herein. In some embodiments, the hybridizing probe 4870 is specific for a target organism, nucleic acid, and/or amplicon. The bonding of the hybridizing probe 4870 to the detection surface 4821 can be performed using any suitable procedure or mechanism. For example, in some embodiments, the hybridizing probe 4870 can be covalently bound to the detection surface 4821.

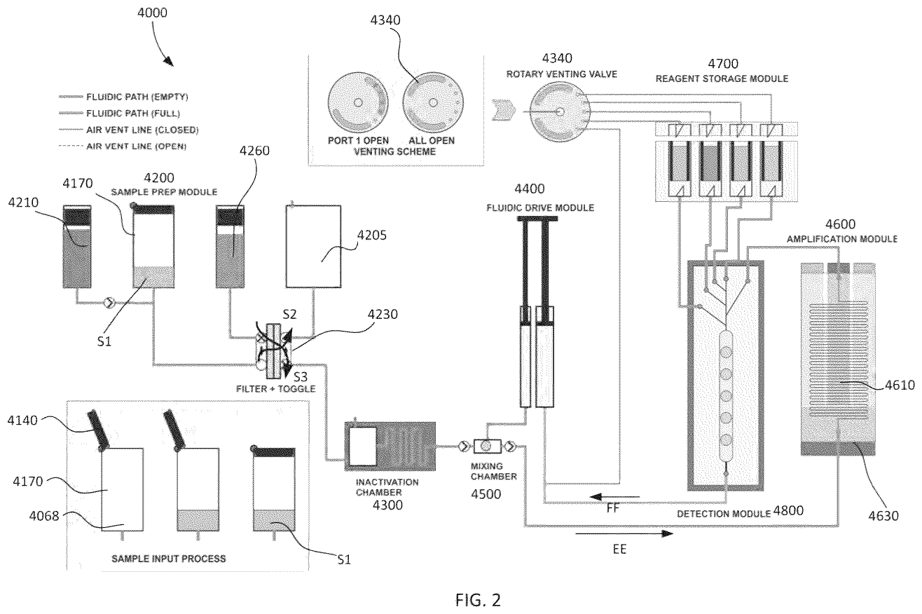

Reference S3 illustrates the biotinylated amplicon that is produced from the amplification step such as, for example, by the amplification module 4600 of FIG. 2 (or any other amplification modules described herein). The biotin can be incorporated within the amplification operation and/or within the amplification module 4600 in any suitable manner. As shown by the arrow XX, the output from the amplification module, including the biotinylated amplicon S3 is conveyed within the read lane and across the detection surface 4821. The hybridizing probe 4870 is formulated to hybridize to the target amplicon S3 that is present within the flow channel and/or in proximity to the detection surface 4821. The detection module 4800 and/or the detection surface 4821 is heated to incubate the biotinylated amplicon S3 in the read lane in the presence of the hybridizing probe 4870 for a few minutes allowing binding to occur. In this manner, the target amplicon S3 is captured and/or is affixed to the detection surface 4821, as shown. Although disclosed as being labeled with biotin, in other embodiments, the target molecules can be labeled in any suitable manner that will allow binding of the complex comprising a sample molecule binding moiety and an enzyme capable of facilitating a colorimetric reaction. For example, in some embodiments, the target molecules can be labeled with one or more of the following: streptavidin, fluorescein, Texas Red, digoxigenin, or Fucose.

In some embodiments, a first wash solution (not shown in FIG. 1) can be conveyed across the detection surface 4821 and/or within the flow channel to remove unbound PCR products and/or any remaining solution. In other embodiments, however, no wash operation is conducted.

As shown by the arrow YY, a detection reagent R5 is conveyed within the read lane and across the detection surface 4821. The detection reagent R5 can be any of the detection reagents described herein. In some embodiments, the detection reagent R5 can be a horseradish peroxidase (HRP) enzyme (“enzyme”) with a streptavidin linker. In some embodiments, the streptavidin and the HRP are cross-linked to provide dual functionality. As shown, the detection reagent is bound to the captured amplicon S3. The detection module 4800 and/or the detection surface 4821 is heated to incubate the detection reagent R5 within the read lane in the presence of the biotinylated amplicon S3 for a few minutes to facilitate binding.

In some embodiments, a second wash solution (not shown in FIG. 1) can be conveyed across the detection surface 4821 and/or within the flow channel to remove unbound detection reagent R5. In other embodiments, however, no second wash operation is conducted.

As shown by the arrow ZZ, a detection reagent R6 is conveyed within the read lane and across the detection surface 4821. The detection reagent R6 can be any of the detection reagents described herein. The detection reagent R6 can be, for example, a substrate formulated to enhance, catalyze and/or promote the production of the signal OP1 when reacted with the detection reagent R5. Specifically, the substrate is formulated such that upon contact with the detection reagent R5 (the HRP/streptavidin) color molecules are produced. As such, a colorimetric output signal OP1 is developed where HRP attaches to the amplicon. The color of the output signal OP1 indicates the presence of bound amplicon: if the target pathogen, target amplicon and/or target organism is present, the color product is formed, and if the target pathogen, target amplicon and/or target organism is not present, the color product does not form.

In some embodiments the detection reagent R6 can be continuously flowed across the detection surface 4821 to ensure that the reaction producing the color molecules does not become limited by the availability of the detection reagent. Moreover, in some embodiments, the detection reagent R6 can be a precipitating substrate.

### Stand-Alone Molecular Diagnostic Test Devices

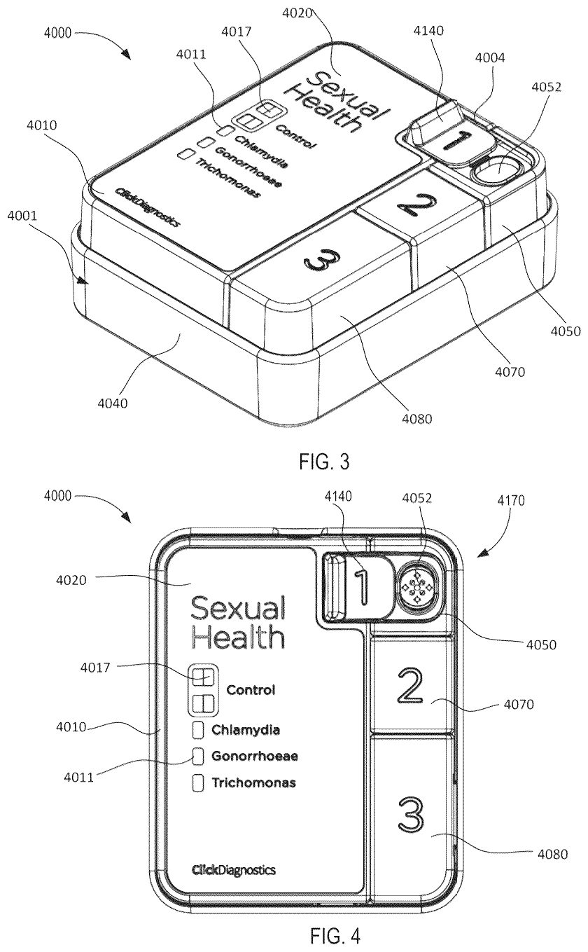

In some embodiments, a method includes lysing a raw sample and performing a reverse transcription polymerase chain reaction (PCR) on the lysed sample to facilitate detection of target RNA, for example to detect a target virus. To facilitate such methods, in some embodiments, a device can include a reverse transcription module to facilitate such methods of isolating and detecting viruses. As one example, FIG. 2 is a schematic illustration of a molecular diagnostic test device 4000 (also referred to as a “test device” or “device”) that includes a reverse transcription module 4270, according to an embodiment. The schematic illustration describes the primary components of the test device 4000 as shown in FIG. 3.

The test device 4000 is an integrated device (i.e., the modules are contained within a single housing) that is suitable for use within a point-of-care setting (e.g., doctor's office, pharmacy or the like), decentralized test facility, or at the user's home. In some embodiments, the device 4000 can have a size, shape and/or weight such that the device 4000 can be carried, held, used and/or manipulated in a user's hands (i.e., it can be a “handheld” device). A handheld device may have dimensions less than 15 cm×15 cm×15 cm, or less than 15 cm×15 cm×10 cm, or less than 12 cm×12 cm×6 cm. In other embodiments, the test device 4000 can be a self-contained, single-use device. Similarly stated, the test device 4000 is a stand-alone device that includes all necessary substances, mechanisms, and subassemblies to perform any of the molecular diagnostic tests described herein. As such, the device 4000 does not require any external instrument to manipulate the biological samples, and only requires a connection to a power source (e.g., a connection to an A/C power source, coupling to a battery, or the like) to complete the methods described herein. In some embodiments, the test device 4000 can be configured with lock-outs or other mechanisms to prevent re-use or attempts to re-use the device.

Further, in some embodiments, the device 4000 can be a CLIA-waived device and/or can operate in accordance with methods that are CLIA waived. Similarly stated, in some embodiments, the device 4000 (and any of the other devices shown and described herein) is configured to be operated in a sufficiently simple manner, and can produce results with sufficient accuracy to pose a limited likelihood of misuse and/or to pose a limited risk of harm if used improperly. In some embodiments, the device 4000 (and any of the other devices shown and described herein), can be operated by a user with minimal (or no) scientific training, in accordance with methods that require little judgment of the user, and/or in which certain operational steps are easily and/or automatically controlled. In some embodiments, the molecular diagnostic test device 4000 can be configured for long term storage in a manner that poses a limited likelihood of misuse (spoilage of the reagent(s), expiration of the reagents(s), leakage of the reagent(s), or the like). In some embodiments, the molecular diagnostic test device 4000 is configured to be stored for up to about 36 months, up to about 32 months, up to about 26 months, up to about 24 months, up to about 20 months, up to about 48 months, or any values there between.

The test device 4000 is configured to manipulate a biological sample S1 to produce one or more output signals associated with a target cell. Specifically, the device 4000 includes an actuator 4050, a sample preparation (or staging) module 4200, a fluidic drive (or fluid transfer) module 4400, a mixing module 4250, an amplification module 4600, a detection module 4800, a reagent module 4700, a valve 4340, and a power and control module (not shown). The test device and certain components therein can be similar to many of the components of the devices shown and described in U.S. patent application Ser. No. 16/186,067, entitled “Portable Molecular Diagnostic Devices and Methods for the Detection of Target Viruses,” filed Nov. 9, 2018, which is incorporated herein by reference in its entirety.

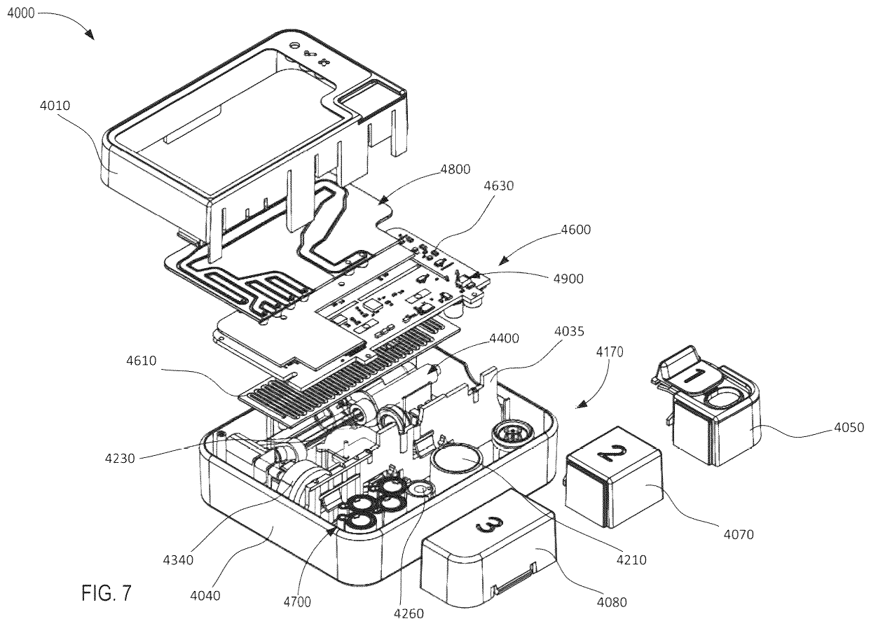

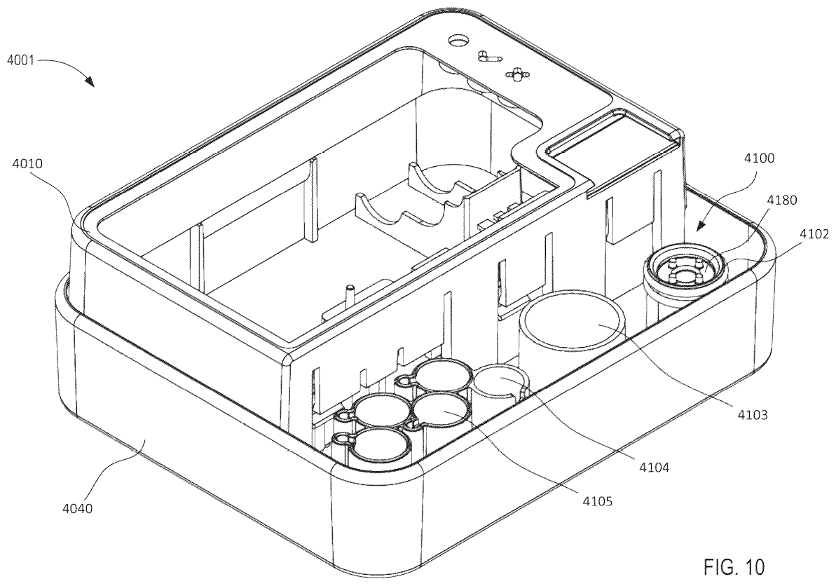

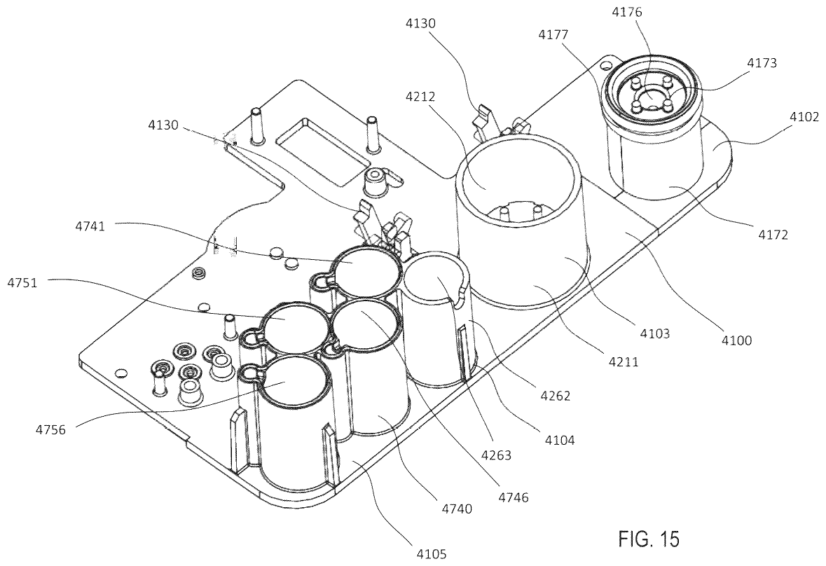

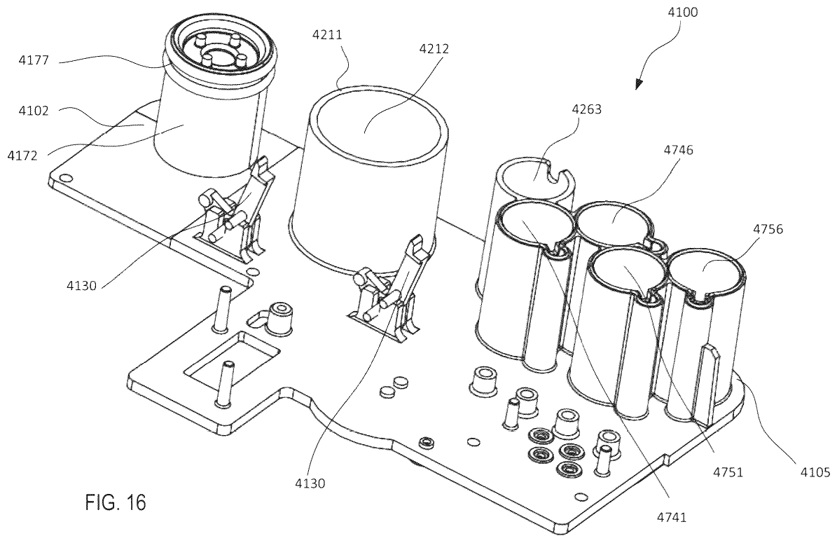

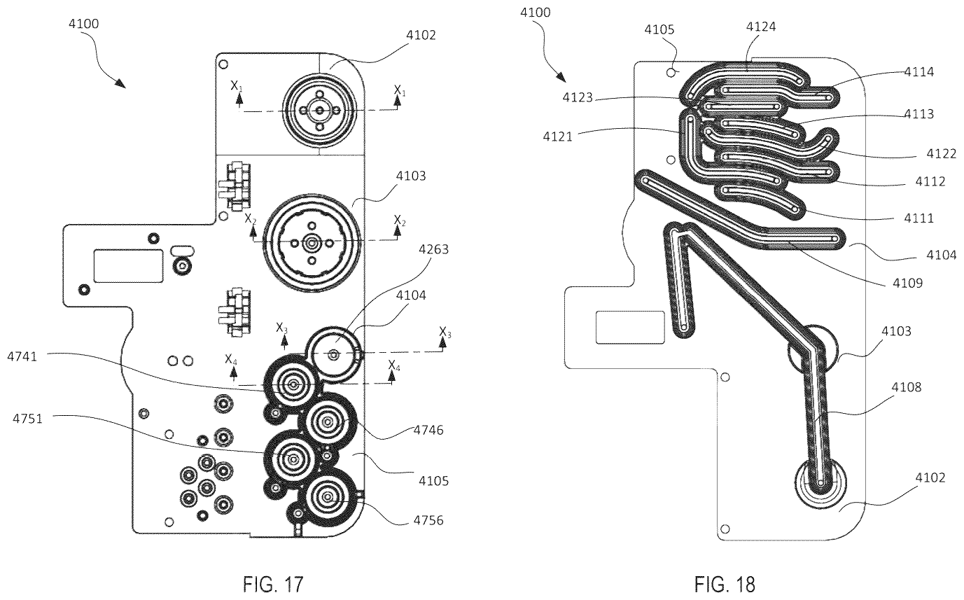

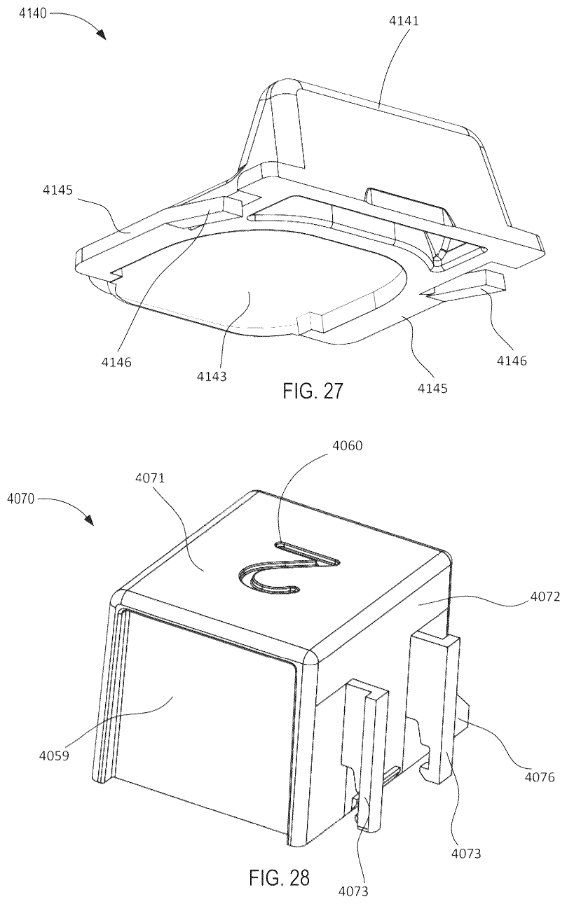

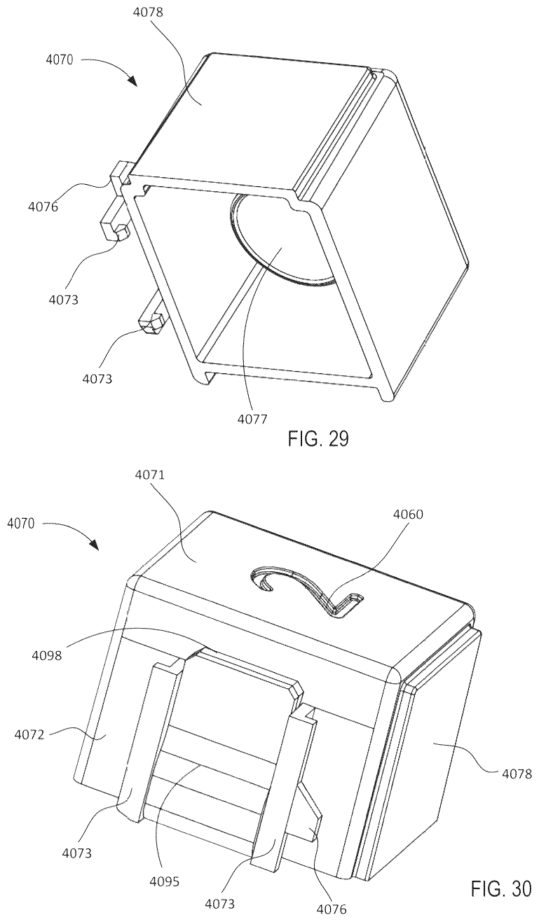

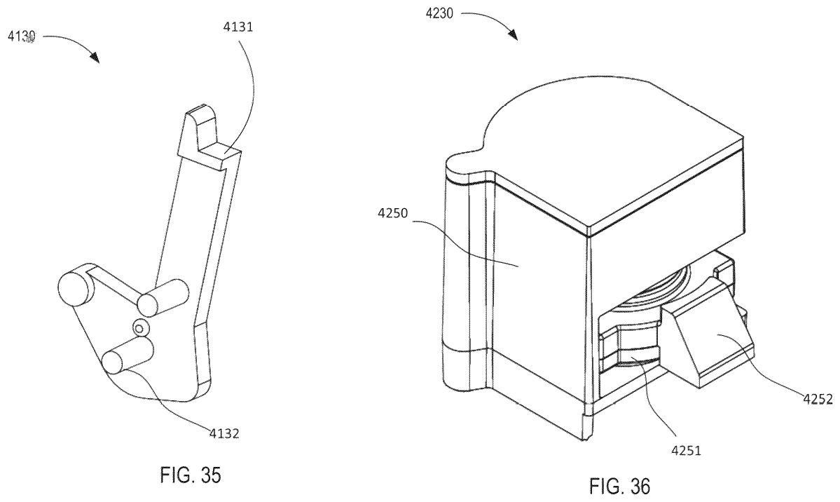

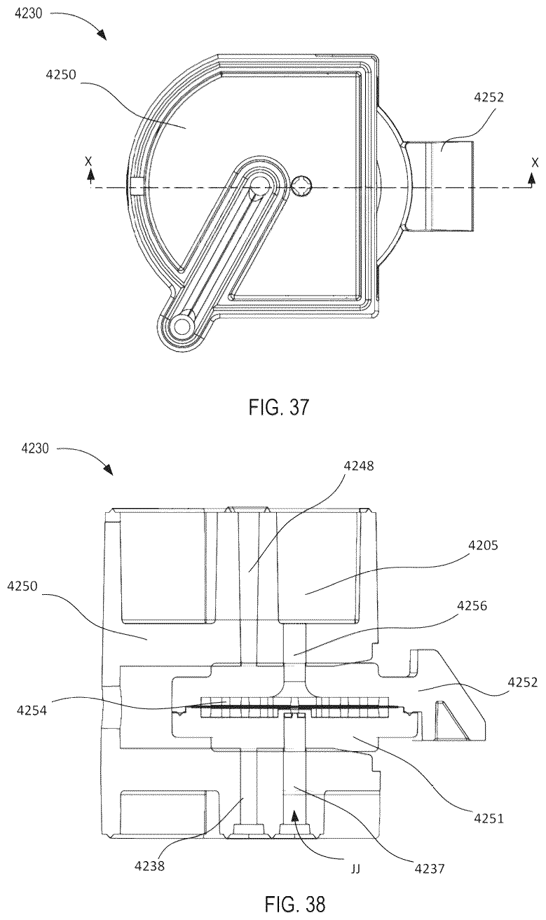

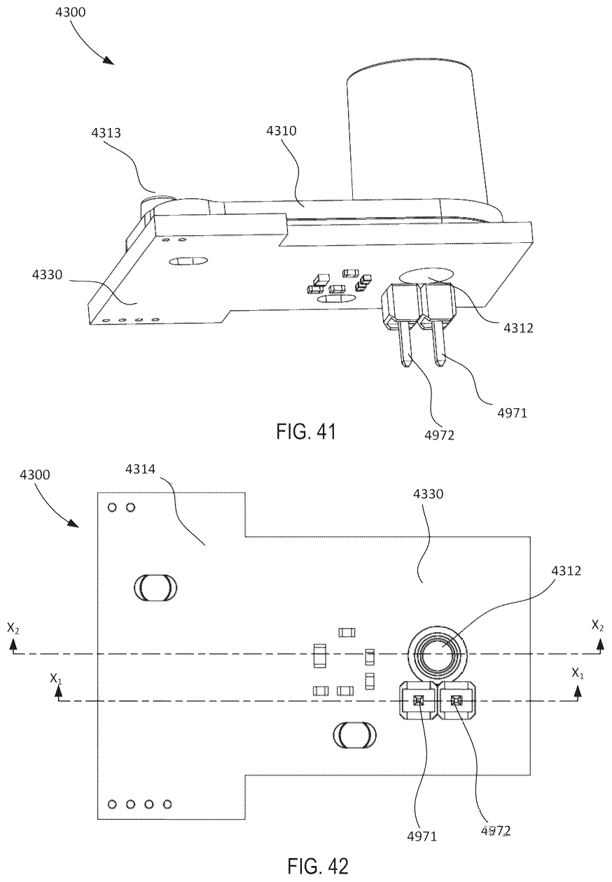

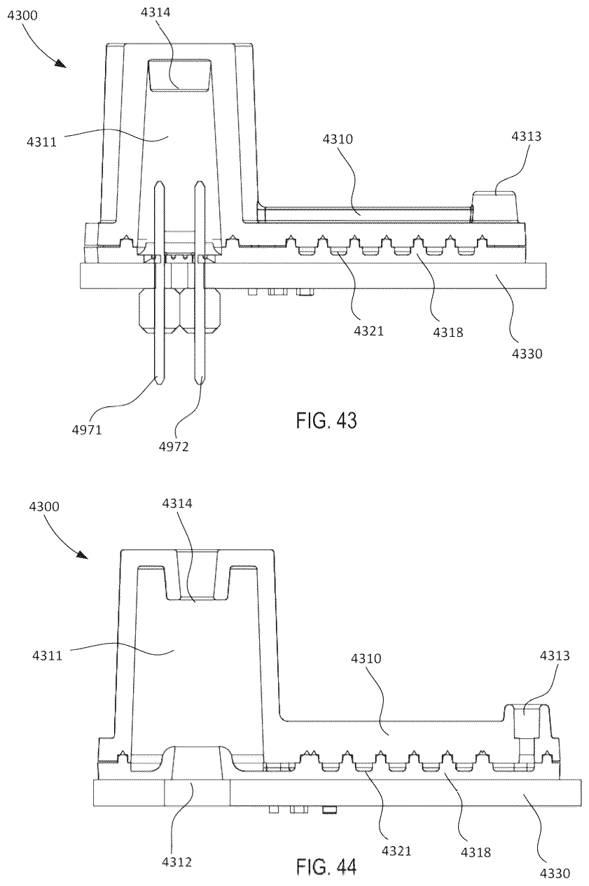

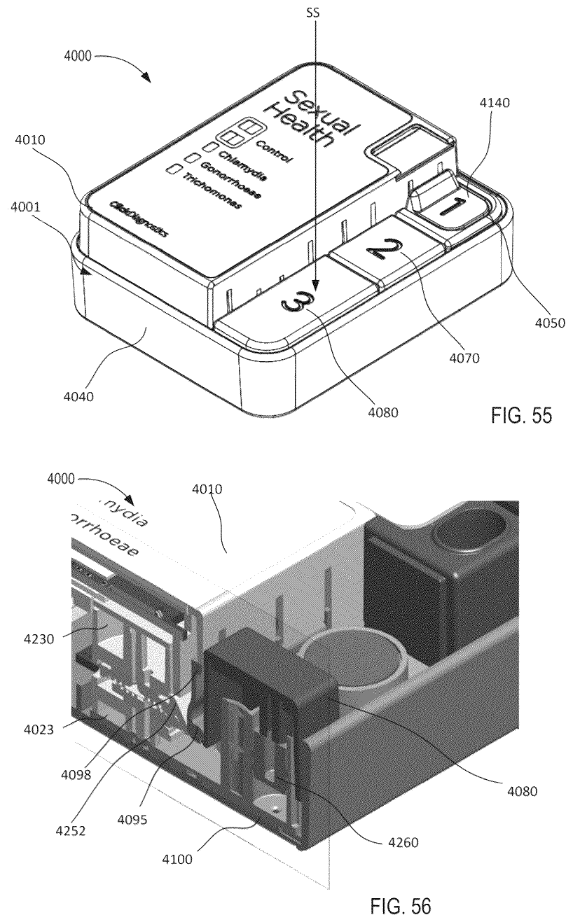

FIGS. 3-58 show various views of the molecular diagnostic test device 4000. The test device 4000 is configured to manipulate an input sample to produce one or more output signals associated with a target cell, according to any of the methods described herein. The diagnostic test device 4000 includes a housing 4001 (including a top portion 4010 and a bottom portion 4030), within which the modules described herein are fully or partially contained. Similarly stated, the housing 4001 (including the top portion 4010 and/or the bottom portion 4030) at least partially surround and/or enclose the modules. As shown in FIG. 7, the device 4000 includes a sample input module 4170, a sample preparation module 4200, a reverse transcription module 4300 (which may also be referred to as a lysing module or an inactivation module, and which may be considered a part of the sample preparation module 4200), a fluidic drive (or fluid transfer) module 4400, a mixing chamber 4500, an amplification module 4600, a detection module 4800, a reagent storage module 4700, a rotary venting valve 4340, and a power and control module 4900. In some embodiments, the sample preparation module 4200 can be considered as including the sample input module 4170 and/or the reverse transcription module (also referred to as the lysing or inactivation module) 4300, but in other embodiments, these modules can be considered as distinct from the sample preparation module 4200. A description of the housing assembly 4001 is followed by a description of each module and/or subsystem.

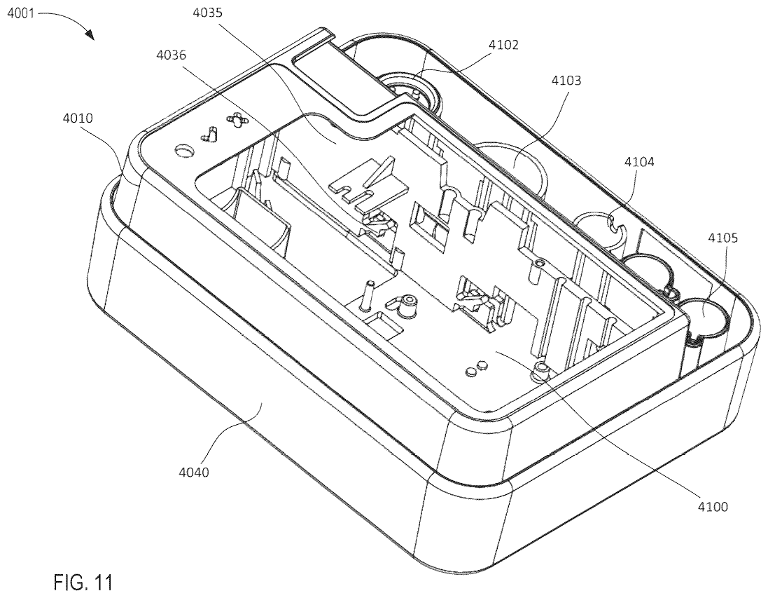

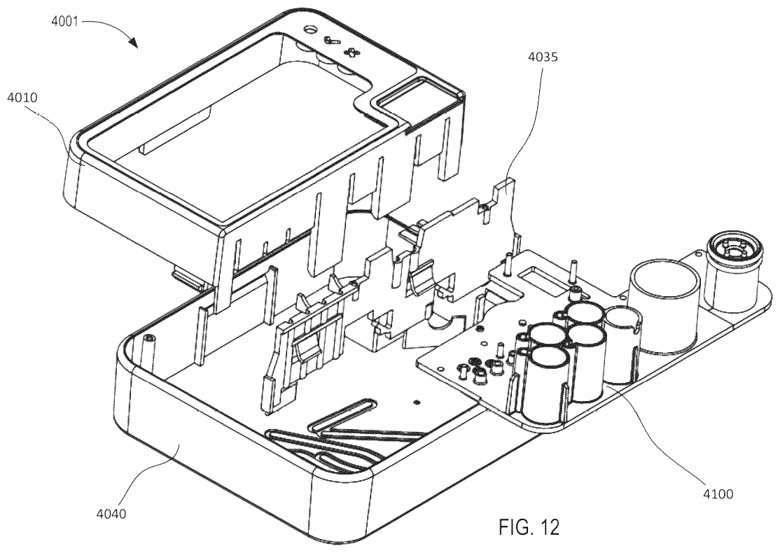

The housing assembly 4001 includes the top housing 4010, the bottom housing 4040, the vertical manifold 4035 (see FIGS. 11 and 12), the sample transfer manifold 4100. As shown, the top housing 4010 includes a label 4020 that defines a series of detection openings 4011 that are aligned with the detection module 4800. In this manner, the signal produced by and/or on each detection surface of the detection module 4800 is visible through the appropriate detection opening 4011. In some embodiments, the top housing 4010 and/or the label 4020 is opaque (or semi-opaque), thereby “framing” or accentuating the detection openings. In some embodiments, for example, the top housing 4010 can include markings 4017 (e.g., thick lines, colors or the like) to highlight the detection opening 4011. For example, in some embodiments, the top housing 4010 can include indicia 4017 identifying the detection opening to a specific disease or target pathogen (e.g., Chlamydia trachomatis (CT), Neisseria gonorrhea (NG) and Trichomonas vagina/is (TV)) or control. In some embodiments, the indicia 4017 can include labels for an of Flu A, Flu B, RSV, or COVID-19. In other embodiments, the top housing 4010 can include a series of color spots having a range of colors associated with a range of colors that is likely produced by the signals produced during the test. In this manner, the housing and/or the label 4020 can contribute to reducing the amount of user judgment required to accurately read the test.

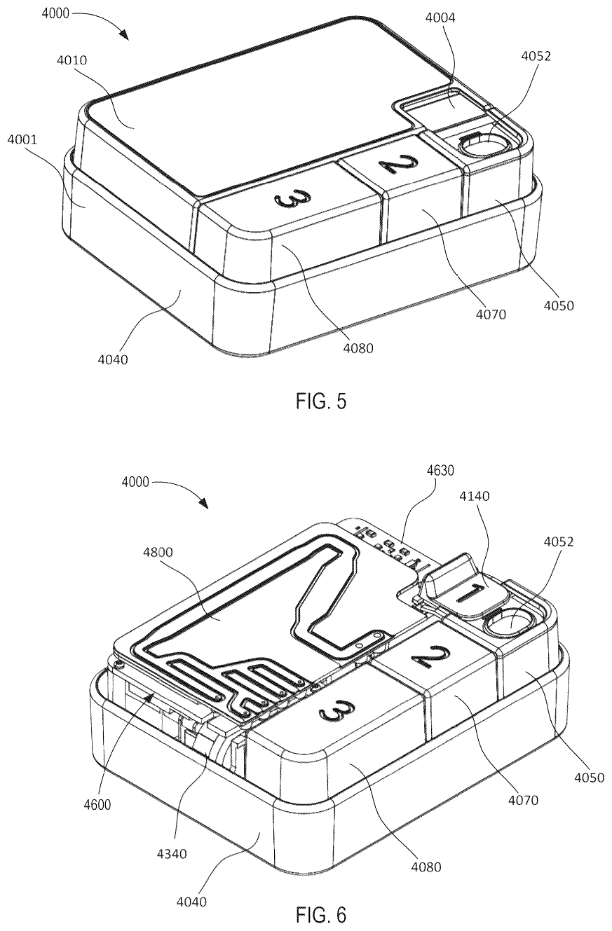

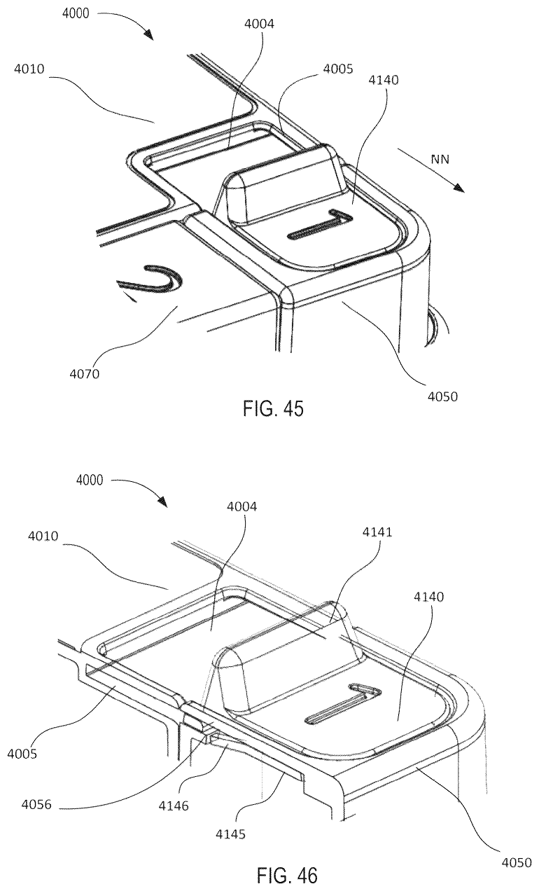

Referring to FIGS. 5, 8, 45 and 46, the top housing 4010 includes a lid portion 4004 to which the sample lid 4140 is movably coupled. The lid portion 4004 includes a recessed surface against which the lid 4140 can be moved, and defines a series of channels 4005. As shown in FIGS. 45 and 46, the channels 4005 slidably receive the rails (also referred to as protrusions) 4145 of the lid 4140. In this manner, as described below, when the lid 4140 is in the first lid (i.e., “opened”) position, the rails 4145 engage a shoulder or lock surface that defines the channels 4005 thereby preventing movement of the lid 4140 and a direction nonparallel to the direction shown by the arrow NN in FIG. 45. In this manner, the top housing 4010 includes a lock surface to which the lid 4140 engages to prevent downward motion of the lid 4140 and the sample input actuator 4050 when the lid 4140 is in the opened position.

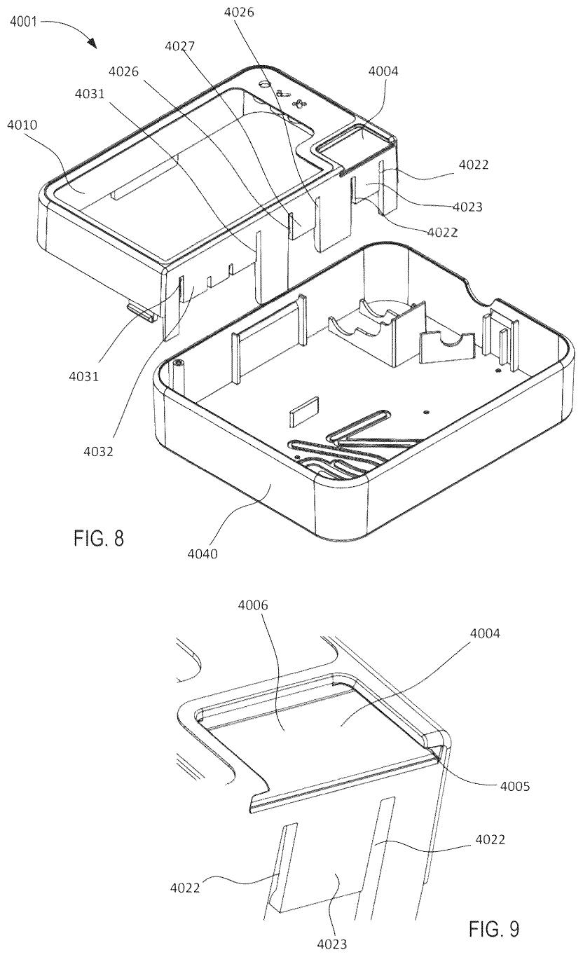

Referring to FIG. 8, the top housing 4010 defines a series of actuator guide slots, each pair of which engages one of the actuator buttons, as described herein. In particular, the top housing 4010 defines a first pair of actuator guide slots 4022 that slidingly receive the mounting protrusions 4063 of the first (or sample input) actuator 4050. The top housing 4010 includes a lock protrusion 4023 between the two guide slots 4022 that engages the spring clip 4095 coupled to the first actuator 4050 when the first actuator 4050 is in its second (or actuated) position. In this manner, the top housing 4010 includes a lock surface (i.e., the lock protrusion 4023) that maintains the first actuator 4050 in its second (or actuated) position to prevent reuse of the diagnostic device 4000, transfer of additional samples into the sample input module 4170, or attempts to actuate the first actuator 4050 multiple times. The top housing 4010 defines a second pair of actuator guide slots 4026 that slidingly receive the mounting protrusions 4073 of the second (or wash) actuator 4070. The top housing 4010 includes a lock protrusion 4027 between the two guide slots 4026 that engages the spring clip 4095 coupled to the second actuator 4070 when the second actuator 4070 is in its second (or actuated) position. In this manner, the top housing 4010 includes a lock surface (i.e., the lock protrusion 4027) that maintains the second actuator 4070 in its second (or actuated) position to prevent reuse of the diagnostic device 4000, or attempts to actuate the second actuator 4070 multiple times. The top housing 4010 defines a third pair of actuator guide slots 4031 that slidingly receive the mounting protrusions 4083 of the third (or reagent) actuator 4080. The top housing 4010 includes a pair of lock protrusions 4032 between the two guide slots 4031 that engage the spring clips 4095 coupled to the third actuator 4080 when the third actuator 4080 is in its second (or actuated) position. In this manner, the top housing 4010 includes a lock surface (i.e., the lock protrusions 4032) that maintain the third actuator 4080 in its second (or actuated) position to prevent reuse of the diagnostic device 4000, or attempts to actuate the third actuator 4080 multiple times.

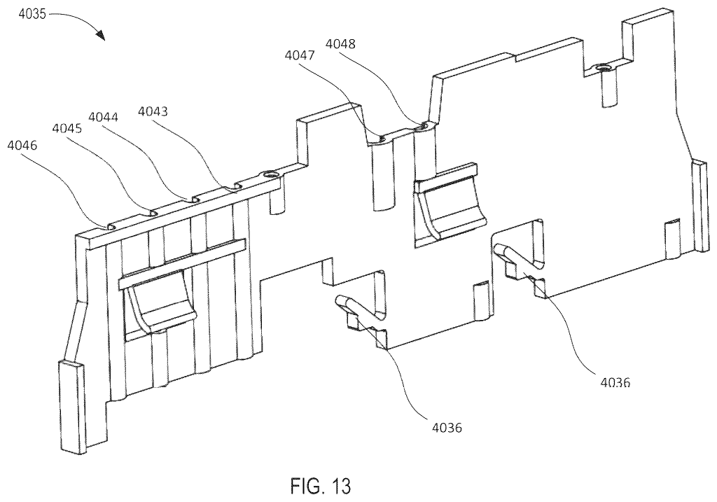

Referring to FIG. 13, the housing assembly 4001 includes the vertical manifold 4035, which provides both structural support and defines flow paths for various fluids that are conveyed within the device 4000. In particular, the vertical manifold 4035 includes two lock arms 4036. As shown in FIG. 11, the lock arms 4036 are positioned against the reagent locks (or lock levers) 4130 (see FIGS. 48 and 50) to maintain the reagent locks 4130 in their locked positions. The vertical manifold 4035 defines a series of reagent passages through which reagents are conveyed from the reagent module 4700 to the detection module 4800. Specifically, the vertical manifold 4035 defines a first reagent passage 4043 through which a first reagent (e.g., a wash) can flow into the detection module 4800, a second reagent passage 4044 through which a second reagent (e.g., a detection reagent) can flow into the detection module 4800, a third reagent passage 4045 through which a third reagent (e.g., a detection reagent, such as a substrate) can flow into the detection module 4800, and a fourth reagent passage 4046 through which a fourth reagent (e.g., a second volume of a detection reagent, such as a substrate) can flow into the detection module 4800. Additionally, the vertical manifold 4035 defines a first vent passage 4047 and a second vent passage 4048.

Although described as being in a particular order or defining a passage through which a particular reagent can flow, any of the reagents described herein can be conveyed into the detection module 4800 via any of the reagent passages. Moreover, in some embodiments, two successive volumes of the same reagent can be conveyed into the detection module via two different reagent passages. For example, in some embodiments, a substrate reagent formulated to enhance, catalyze and/or promote the production of the signal OP1 from the detection reagent (e.g., similar to the reagent R2 described above) can be stored in two independent volumes. In use, a first volume of the substrate reagent can be conveyed into the detection module 4800 via the third reagent passage 4045 at a first time, and a second volume of the substrate reagent can be conveyed into the detection module 4800 via the fourth reagent passage 4046 at a second time.

The housing assembly 4001 includes the sample transfer manifold 4100, which provide both structural support and defines flow paths for various fluids that are conveyed within the device 4000. In particular, the sample transfer manifold 4100 includes a sample input portion 4102, a wash portion 4103, an elution portion 4104, and a reagent portion 4105. Details of each of these portions is discussed below in conjunction with the various modules of the device 4000.

The sample preparation module 4200 includes a sample input module 4170, a wash module 4210, an elution module 4260, a filter assembly 4230, and various fluidic conduits (e.g., tubes, lines, valves, etc.) connecting the various components. The device 4000 also includes the reverse transcription module 4300, which, together with the sample preparation module 4200, performs the nucleic acid extraction and reverse transcription (to produce complementary DNA, or cDNA) according to any of the methods described herein. Thus, although the sample preparation module 4200, the sample input module 4170, and the reverse transcription module 4300 are described as separate modules, in other embodiments, the structure and function of the sample preparation module 4200 can be included within or performed by the reverse transcription module 4300 and/or the sample input module 4170, and vice-versa. Similarly stated, any of the sample input modules, sample preparation modules, lysing modules, reverse transcription modules, and/or inactivation modules described herein can include any of the structure and/or perform any of the functions of the other modules to perform any of the methods of sample preparation, nucleic acid extraction, and/or reverse transcription described herein. By eliminating the need for external sample preparation and a cumbersome instrument, the device 4000 is suitable for use within a point-of-care setting (e.g., doctor's office, pharmacy, or the like), at a decentralized location (e.g., an office building, a public forum, or the like) or at the user's home, and can receive any suitable biological sample S1. The biological sample S1 (and any of the input samples described herein) can be, for example, blood, urine, male urethral specimens, vaginal specimens, cervical swab specimens, and/or nasal swab specimens gathered using a commercially available sample collection kit.

Moreover, although the sample preparation module 4200 is shown and described as including a wash module 4210, an elution module 4260, and a filter assembly 4230, in other embodiments the sample preparation module 4200 need not include any of a wash module 4210, an elution module 4260, or a filter assembly 4230. For example, in some embodiments, the sample preparation module 4200 can be devoid of a filter assembly.

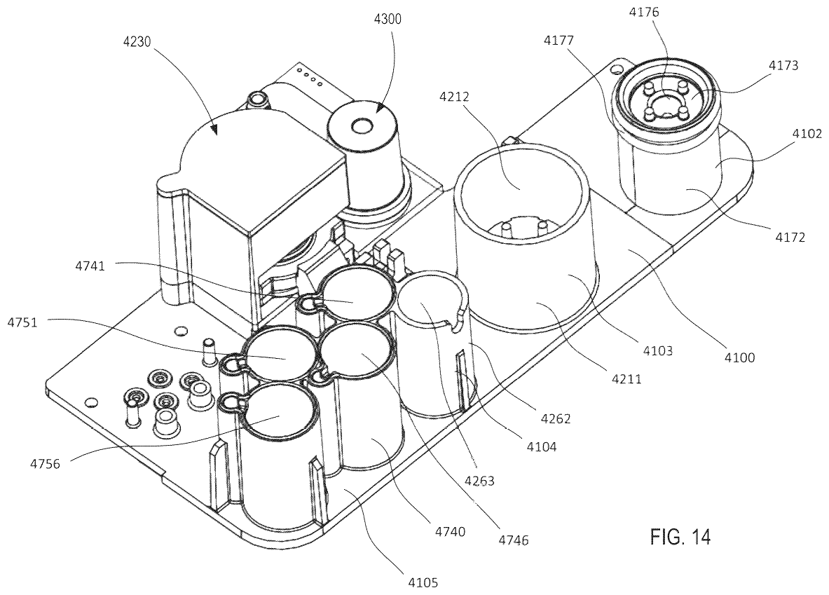

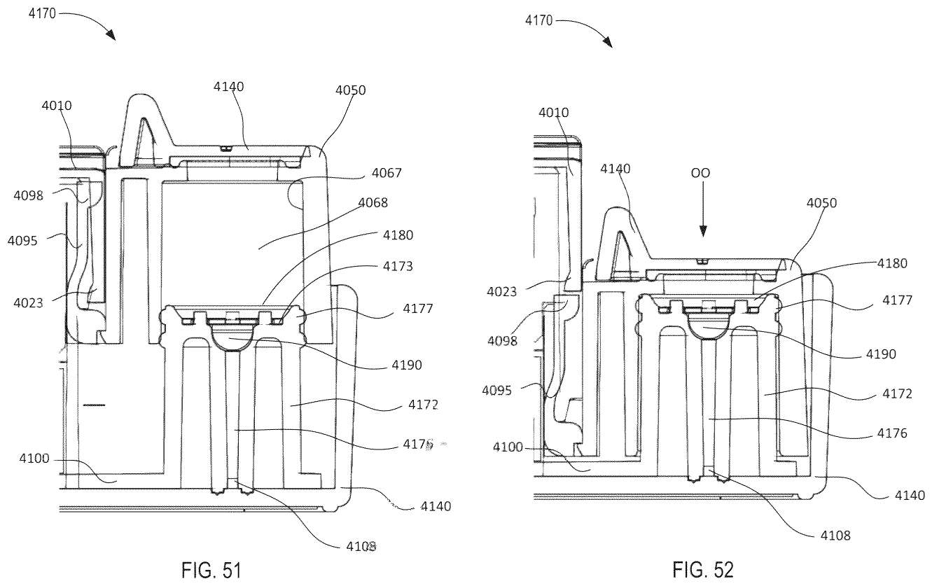

The sample input module 4170 is configured to receive a biological sample S1 containing a biological entity, and convey the biological sample toward the remaining elements of the sample preparation module 4200 (e.g., the filter assembly 4230). The biological sample S1 can be any of the sample types described herein, and the biological entity can be any of the entities described herein. In some embodiments, the biological sample S1 can be a combined (or pooled) sample that includes multiple individual samples (e.g., from multiple different patients). The sample input module 4170 includes the sample input portion 4102 of the sample transfer manifold 4100, the sample input (or first) actuator 4050, and the lid 1140. Referring to FIGS. 14-19, the sample input portion 4102 of the sample transfer manifold 4100 includes a cylindrical housing 4172 and a cover 4180. As shown in FIGS. 51 and 52, the top surface of the cylindrical housing 4172 (including the top surface 4173 and/or portions of the cover 4180) and the inner surface 4067 of the first actuator 4050 define a sample input volume 4068, within which the biological sample is conveyed at the start of a test. The outer portion of the cylindrical housing 4172 includes one or more seals 4177 slidingly engage the inner surface 4067 of the first actuator 4050 to form a fluid-tight seal.

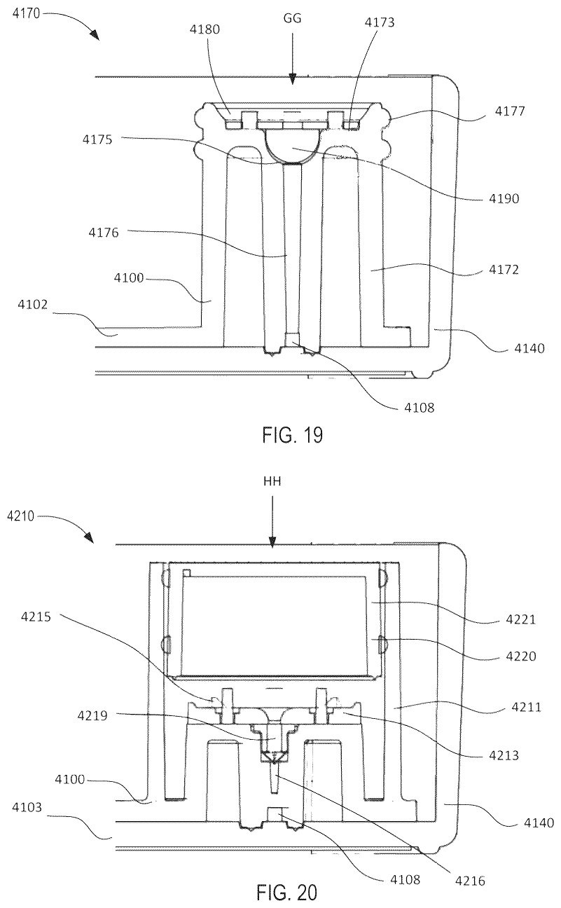

The cylindrical housing 4172 includes the top surface 4173 (see FIG. 19) that defines a reagent volume 4175 within which one or more lyophilized reagents 4190 are contained. In particular, the lyophilized bead 4190 (which is also referred to herein as a pellet) is retained within the reagent volume 4175 by the cover 4180, which defines a series of openings. In this manner, the biological sample can be conveyed through the openings of the cover 4180 and into the reagent volume 4175 to mix with and reconstitute the lyophilized reagent 4190. In other embodiments, the lyophilized reagent is applied via a dry reagent coating process to one or more surfaces within the reagent volume. Thus, the sample input module 4170 functions both to convey the sample into the device and also to ensure that the desired reagents are mixed into the biological sample.

The lyophilized reagent 4190 can be any of the reagents described herein. In some embodiments, the lyophilized reagent 4190 can be a positive control organism, such as Aliivibrio fischeri, N. subflava, or any other suitable organism. Specifically, Aliivibrio fischeri is suitable because it is gram negative, nonpathogenic, bio safety level 1, not harmful to the environment, and is extremely unlikely to be found on a human. The positive control surface within the detection module contains capture probes for both the control organism (e.g., A. fischeri) as well as each of the target organisms. This arrangement ensures that the positive control surface always produces color if the device functions correctly. If only the control organism were present, a very strong positive for one of the target organisms could “swamp out” or “outcompete” the amplification of the control organism during PCR. Under such circumstances, the positive control spot would not produce a color change which would be confusing for the user. This arrangement facilitates the detection method and the device 4000 being operated by a user with minimal (or no) scientific training, in accordance with methods that require little judgment.

In other embodiments, the lyophilized reagent 4190 can be any of the reducing agents described herein. For example, in some embodiments, the lyophilized reagent 4190 can be lithium, sodium, potassium, aluminum amalgam, lithium aluminum hydride, sodium borohydride, sodium cyanoborohydride, lithium tri-butoxy aluminum hydride, dibutoxy aluminum hydride, lithium triethyl borohydride, tris(2-carboxyethyl)phosphine (TCEP), dithiothreitol (DTT), mercaptoethanol, cysteine, thioglycerol, thioglycolic acid, cysteamine, glutathione, N-acetylcysteine (NAC), β-mercaptoethanol, sodium dodecyl sulfate (SDS), sodium sulfite, ammonium sulfamate, sodium bisulfite, dithionite metabisulfite sulfur dioxide, vitamin C, ascorbic acid, and dimethylsulfoxide (DMSO). In some embodiments, the lyophilized reagent can be N-acetylcysteine (NAC). In some embodiments, the N-acetylcysteine (NAC) is N-acetyl-L-cysteine. In some embodiments, the N-acetylcysteine is N-acetyl-D-cysteine.

The cylindrical housing 4172 defines a first (or vertical) fluid passage 4176 that is between (and fluid communication with) a sample input passage 4108 defined by the sample transfer manifold 4100 (see also FIG. 18, which shows the various fluid passages). The sample input passage 4108 is in fluid communication with the wash module 4210 and the filter assembly 4230. In this manner, when the biological sample is compressed by the first actuator 4050 it is conveyed from the sample input volume 4068, through the reagent volume 4175, through the first fluid passage 4176 and into the sample input passage 4108, as shown by the arrow GG in FIG. 19. In some embodiments, the sample is conveyed in this manner to the filter assembly 4230.

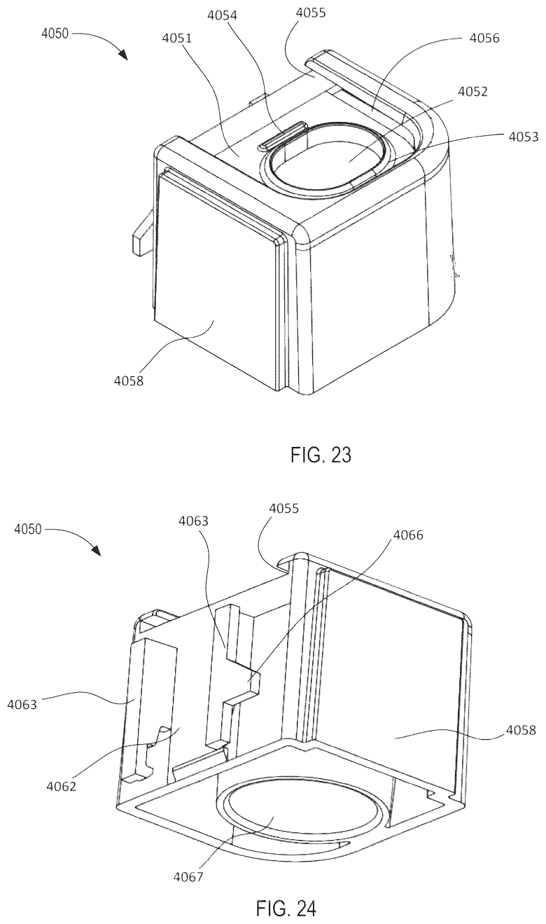

Referring to FIGS. 23-25, the sample input (or first) actuator 4050 includes a top surface 4051, a side surface 4058, a back surface 4062, and an inner surface 4067. The top surface 4051 defines an opening 4052 into the sample input volume 4068. The top surface 4051 also includes a seal 4053 surrounding the opening 4052. In this manner, when the lid 4140 is in its first lid position (opened), the biological sample can be conveyed through the opening 4052 and into the sample input volume 4068. The lid 4140 can then be sealed about the opening. The seal 4053 can be an elastomeric seal, such as an O-ring or the like, and can produce a substantially fluid-tight seal to protect against spilling or leaking during the test. The seal 4053 also fluidically isolates the sample input volume 4068 such that the pressure generated therein can be maintained to convey the biological sample through the passage 4108 (rather than leaking out via the opening 4052).

The top surface 4051 includes a protrusion 4054 and defines two side slots 4055 and two lock openings 4056. The protrusion 4054 is in contact with the end of the lid 4140 when the lid 4140 is in its opened position. In this manner, the protrusion 4054 acts as a detent to limit movement of the lid 4140 from its opened position (FIG. 3) to its closed position (FIG. 45). Similarly stated, the protrusion 4054 can offer resistance against the movement of the lid 4140 towards the second (or closed) position, thereby limiting the likelihood that a user will inadvertently close the lid 4140 before desired.

The side slots 4055 are aligned with the channels 4005 of the top housing 4010, and slidably receive the rails (also referred to as protrusions) 4145 of the lid 4140. In particular, when the lid 4140 is moved from the first lid position (opened) to the second lid position (closed), the rails 4145 move from the channels 4005 and into the side slots 4055 of the first actuator 4050. When the lid 4140 is in the opened position, because the rails 4145 are disengaged from the top housing 4010, the lid 4140 (and therefore the first actuator 4050) are free to move relative to the top housing 4050. Said another way, when the lid 4140 is in the closed position, the lid 4140 is sealed about the opening 4052 and the first actuator 4050 can be moved from its first position to its second position. The two lock openings 4056 are “through openings” beneath two side slots 4055, and define a volume within which the lock portions 4146 of the lid 4140 can expand (or deform) when the lid 4140 is in the closed position (see FIG. 46). In this manner, the lock portions 4146 engage a shoulder that borders the two lock openings 4056 to prevent the lid 4140 from being moved from its closed position back towards its opened position.

The side surface 4058 includes a raised surface that matingly engages with a side surface 4059 of the second (wash) actuator 4070. In this manner, when the second actuator 4070 is moved to its second position, the side surface 4058 of the first actuator 4050 guides the movement of the second actuator 4070. As described above, the inner surface 4067 defines a portion of a boundary of the sample input volume 4068.

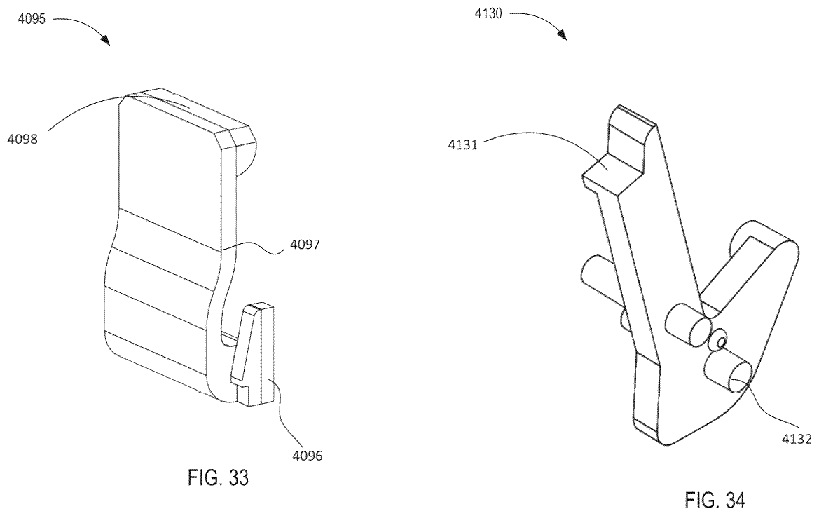

The back surface 4062 includes a pair of mounting protrusions 4063. The mounting protrusions 4063 are received within the actuator guide slots 4022, as described above. Additionally, the mounting protrusions 4063 each include a connection portion that is connected to the base 4096 of the spring clip 4095 (see FIGS. 25 and 33). In particular, the spring clip 4095 is coupled to the mounting protrusions 4063 such that when the first actuator 4050 is in its first position, the lock protrusion 4023 of the housing (i.e., between the two actuator guide slots 4022) is between the spring clip 4095 and the back surface 4062 (see FIG. 51). As shown in FIG. 33, the spring clip 4095 includes a resilient portion 4097 that biases the lock portion 4098 of the spring clip 4095 against the lock protrusion 4023. As shown in FIG. 52, when the first actuator 4050 is moved to its second (or actuated) position, the lock portion 4098 becomes disengaged with the lock protrusion 4023 of the housing, and contacts the back surface 4062 of the first actuator 4050. In this manner, the lock portion 4098 will engage the shoulder of the lock protrusion 4023 if the first actuator 4050 is moved back towards its first position. In this manner, the first actuator 4050 is retained in its second position.

As shown in FIG. 24, the inboard mounting protrusion 4063 (i.e., the protrusion adjacent the second actuator 4070) includes an actuation protrusion 4066. As described in more detail below, the actuation protrusion 4066 is configured to contact the reagent lock 4130 that is in contact with the second actuator 4070 (see FIGS. 48 and 50). When the first actuator 4050 is in its first position, the reagent lock 4130 is in contact with the second actuator 4070, thereby preventing the second actuator 4070 from being moved. When the first actuator 4050 moves from its first position (FIG. 48) to its second position (FIG. 50), the actuation protrusion 4066 causes the reagent lock 4130 to rotate as shown by the arrow PP in FIG. 50 to release the reagent lock 4130 from the second actuator 4070. In this manner, the second actuator 4070 cannot be depressed (or moved) before the first actuator 4050 is moved to its second (or actuated) position.

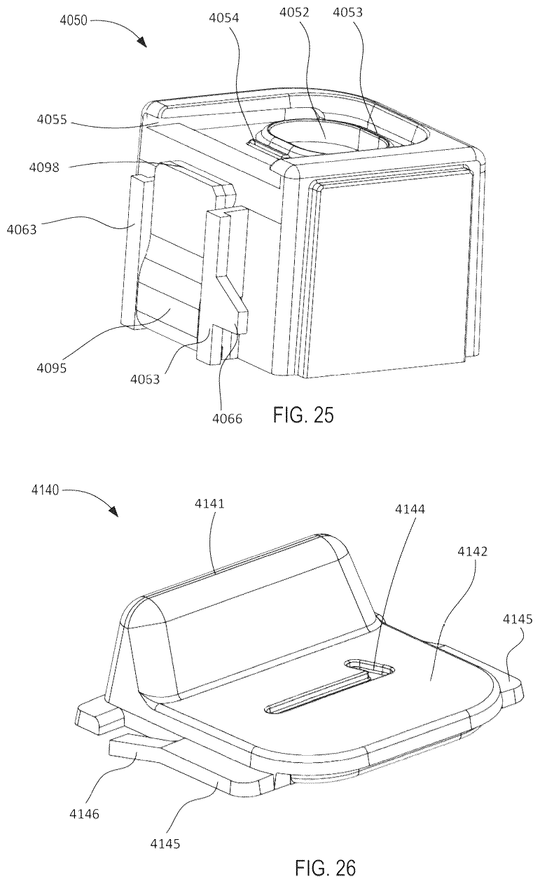

As shown in FIGS. 26 and 27, the lid 4140 includes a top portion 4141, a cover portion 4142, and a pair of side rails 4145. The top portion 4141 extends above the surface of the top housing 4010 and can be manipulated or grasped by the user to move the lid 4140 from the first lid position to the second lid position. The cover portion 4142 includes an indicator 4144 (i.e., “1”) to guide a user in the correct sequence of steps for use of the apparatus 4000. The bottom surface 4143 (or seal surface 4143) of the cover portion 4142 engages the seal 4053 when the lid 4140 is in the closed position to produce a substantially fluid-tight seal to protect against spilling or leaking during the test. As describe above the side rails 4145 each include a lock portion 4146. The lock portions 4146 are deformed when the lid 4140 is in its opened position such that the side rails 4145, including the lock portions 4146, are within the channels 4005 of the top housing 4010. When the lid 4140 is moved to the closed position, the lock portions 4146 expand or deform to their natural condition (as shown) and extend within the two lock openings 4056 to prevent the lid 4140 from being moved from its closed position back towards its opened position (see FIG. 46).

The wash module 4210 is configured to convey a wash solution toward the remaining elements of the sample preparation module 4200 (e.g., the filter assembly 4230). Importantly, as described herein, the wash module 4210 is configured such that it cannot be actuated out of the desired sequence of operations. Specifically, the wash module 4210 is configured to be locked until after the biological sample has been conveyed to the sample preparation module 4200. The wash module 4210 includes the wash portion 4103 of the sample transfer manifold 4100, the wash (or second) actuator 4070, and a wash container 4220. Referring to FIGS. 14-18, and 20, the wash portion 4103 of the sample transfer manifold 4100 includes a cylindrical housing 4211 and atop surface (or cover) 4123.

The upper portion of the cylindrical housing 4211 defines a volume 4212 within which the wash container 4220 is disposed. As shown in FIG. 20, the wash container 4220 includes a side wall 4221 that is sealingly engaged with in inner side wall of the cylindrical housing 4211. The wash container 4220 can include any number of seals (e.g., O-rings, gaskets, or the like) to fluidically isolate the volume 4212 from the external volume of the device 4000. In this manner, the sealed wash container 4220 can limit backflow of the wash solution out of the device 4000. The lower portion of the wash container 4220 is sealed with a frangible member to define a sealed container suitable for long-term storage of the wash solution. As described below, the frangible seals are punctured upon actuation of the wash module 4210 allow the wash solution within the container 4220 to be conveyed to the filter assembly 4230. The frangible seals can be, for example, a heat-sealed BOPP film (or any other suitable thermoplastic film). Such films have excellent barrier properties, which prevent interaction between the fluids within the canister and external humidity, but also have weak structural properties, allowing the films to be easily broken when needed. The wash solution within the wash container 4220 can be any suitable solution as described herein.

The lower portion of the cylindrical housing 4211 defines a second (or vertical) fluid passage 4216 that is between (and fluid communication with) the sample input passage 4108. A check valve 4219 is included within the second passage 4216, and is retained in place by the cover 4123. The check valve 4219 prevents backflow from the sample input passage 4108 upward into the volume 4212 (i.e., in a direction opposite the direction shown by arrow HH). The cover 4123 also includes a series of puncturers 4215 (only one is identified in FIG. 27). In use, when the wash container 4220 is moved downward (in the direction shown by the arrow HH), the puncturers rupture the frangible membrane to release the wash solution from within the wash container 4220. Moreover, the housing 4211 defines an annular groove or opening that receives the side wall of the wash container 4220 after it has been punctured and moved downward. In this manner, the dead volume produced by the interface between the wash container 4220 and the housing 4211 is minimized.

As described above, the sample input passage 4108 extends from the sample input module 4170 to the wash module 4210, and on to the filter assembly 4230. In this manner, after the biological sample is conveyed from the sample input volume 4068 to the filter assembly 4230, the wash solution (described herein) can be conveyed along a portion of the same path (the sample input passage 4108) to the filter assembly 4230. Specifically, the wash solution can be conveyed from the wash container 4220 and through the second fluid passage 4216, as shown by the arrow HH in FIG. 20.

The wash module 4210 is actuated by the wash (or second) actuator 4070. Referring to FIGS. 28-30, the wash (or second) actuator 4070 includes a top surface 4071, a first side surface 4059, a second side surface 4078, a back surface 4072, and an inner surface 4077. The top surface 4071 includes an indicator 4060 (i.e., “2”) to guide a user in the correct sequence of steps for use of the apparatus 4000. The first side surface 4059 includes a recessed area that matingly engages with the side surface 4058 of the first actuator 4050. In this manner, when the second actuator 4070 is moved to its second position, the side surface 4058 of the first actuator 4050 guides the movement of the second actuator 4070. The second side surface 4078 includes a raised surface that matingly engages with a side surface 4090 of the third (reagent) actuator 4080. In this manner, when the third actuator 4080 is moved to its second position, the side surface 4078 of the second actuator 4070 guides the movement of the third actuator 4080. The inner surface 4077 includes a protrusion that engages the side wall of the wash container 4220 and moves the wash container 4220 against the puncturers 4215, as described herein.

The back surface 4072 includes a pair of mounting protrusions 4073. The mounting protrusions 4073 are received within the actuator guide slots 4026. Additionally, the mounting protrusions 4073 each include a connection portion that is connected to the base 4096 of the spring clip 4095 (see FIGS. 30 and 33). In particular, the spring clip 4095 is coupled to the mounting protrusions 4073 such that when the second actuator 4070 is in its first position, the lock protrusion 4027 of the housing (i.e., between the two actuator guide slots 4026) is between the spring clip 4095 and the back surface 4072 (similar to the arrangement shown in FIG. 51). As shown in FIG. 33, the spring clip 4095 includes a resilient portion 4097 that biases the lock portion 4098 of the spring clip 4095 against the lock protrusion 4027. Similar to the arrangement shown in FIG. 52, when the second actuator 4070 is moved to its second (or actuated) position, the lock portion 4098 becomes disengaged with the lock protrusion 4027 of the housing, and contacts the back surface 4072 of the second actuator 4070. In this manner, the lock portion 4098 will engage the shoulder of the lock protrusion 4027 if the second actuator 4070 is moved back towards its first position. In this manner, the second actuator 4070 is retained in its second position.

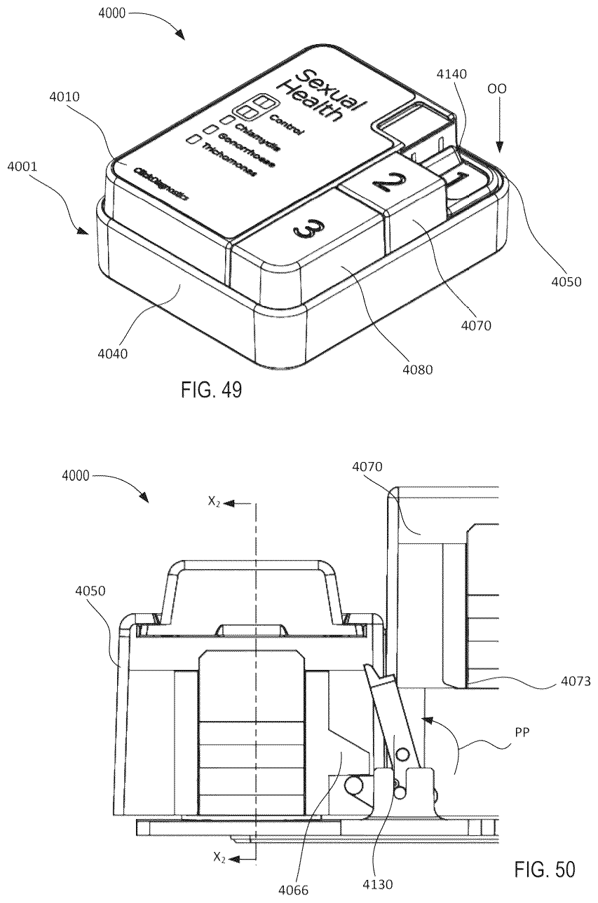

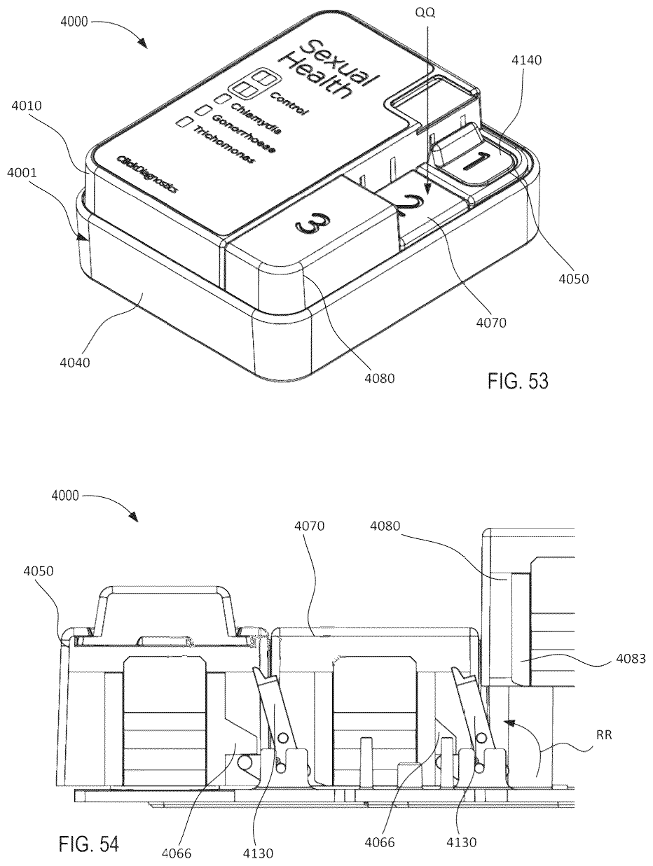

As shown in FIG. 30, the inboard mounting protrusion 4073 (i.e., the protrusion adjacent the third actuator 4080) includes an actuation protrusion 4076. As described in more detail below, the actuation protrusion 4076 is configured to contact the reagent lock 4130 that is in contact with the third actuator 4080 (see FIGS. 48 and 54). Thus, when the second actuator 4070 is in its first position, the reagent lock 4130 is in contact with the third actuator 4080, thereby preventing the third actuator 4080 from being moved. When the second actuator 4070 moves from its first position (FIG. 49) to its second position (FIG. 53), the actuation protrusion 4076 causes the reagent lock 4130 to rotate as shown by the arrow RR in FIG. 54 to release the reagent lock 4130 from the third actuator 4080. In this manner, the third actuator 4080 cannot be depressed (or moved) before the first actuator 4050 and the second actuator 4070 have both been actuated.

As described herein, the biological sample and the wash solution are conveyed through the optional filter assembly 4230. The filter assembly is configured to receive an elution buffer (via a backflush operation) to convey the desired particles (and the elution buffer) to the lysing (or reverse transcription) module 4300. The filter assembly 4230 is shown in FIGS. 36-39. The filter assembly 4230 includes a filter housing 4250, a first plate 4251, a second plate 4252, and a filter membrane 4254. As described herein, the filter assembly 4230 is configured to filter and prepare the input sample (via the sample input operation and the sample wash operation), and to allow a back-flow elution operation to deliver captured particles from the filter membrane 4254 and deliver the eluted volume to the target destination (e.g., towards the amplification module 4600).

The filter housing 4250 contains the first plate 4251 and the second plate 4252, with the filter membrane 4254 disposed therebetween. As described herein, the second plate 4252 can move relative to the first plate 4251 to allow the filter to toggle between various flow conditions. The filter housing 4250 defines a sample inlet port 4237, a sample outlet port 4238, an elution inlet port 4248, and a waste reservoir 4205. The sample inlet port 4237 allows communication from the sample input passage 4108 and through the filter membrane 4254, as shown by the arrow JJ in FIG. 38. When the filter assembly 4230 is in its first configuration (i.e., the “sample flow” configuration, see FIG. 38), the flow port 4256 of the second plate 4252 is in fluid communication with the waste reservoir 4205. Thus, when the filter assembly 4230 is in its first configuration, the sample and wash solutions flow (towards the waste reservoir 4205), as indicated by the arrow JJ in FIG. 38.

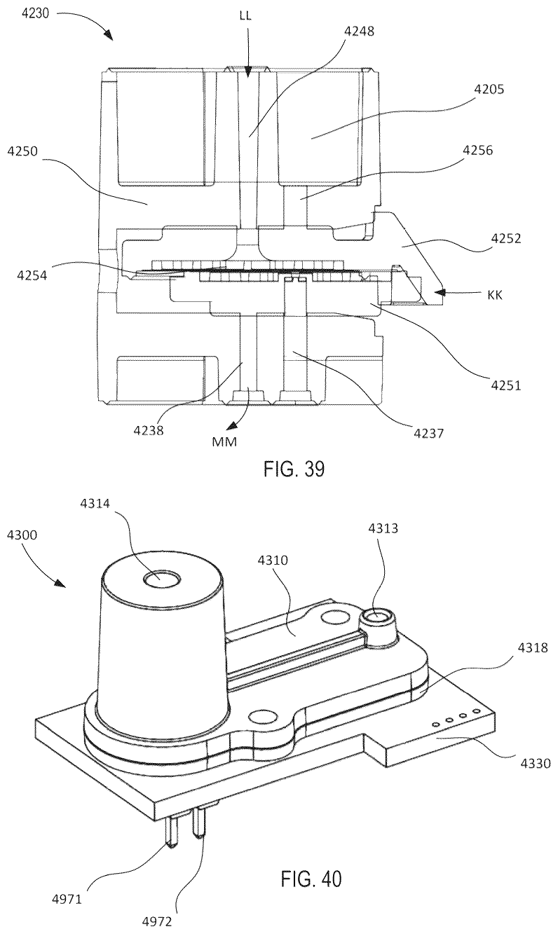

After the wash operation, the filter assembly is moved from its first configuration to its second configuration (FIG. 39). Specifically, the second plate 4252 is moved relative to the first plate 4251, as shown by the arrow KK in FIG. 39. The second plate 4252 includes a tapered actuation surface that is moved when the third actuator 4080 is moved from its first position to its second position. As shown in FIG. 56, the back surface 4082 and/or the spring clips 4095 of the third actuator 4080 contact the tapered surface and move the second plate 4252 to transition the filter assembly 4230 to its second configuration. When the filter assembly 4230 is in its second configuration (i.e., the “elution flow” configuration, see FIG. 39), the flow port 4256 of the second plate 4252 is in fluid communication with the elution inlet port 4248. Thus, when the filter assembly 4230 is in its second configuration, the elution buffer can flow from the elution module 4260 (described below) and through the filter membrane 4254, as indicated by the arrow LL in FIG. 39. The elution buffer and the captured particles flow out of the filter assembly 4230 and toward the reverse transcription module 4300 via sample outlet port 4238.

The elution module (or assembly) 4260 of the sample preparation module 4200 is contained within the housing, and defines an elution volume within which an elution composition is stored. The elution composition can be any of the elution compositions described herein. In some embodiments, the elution composition can include proteinase K, which allows for the release of any bound cells and/or nucleic acid molecules (e.g., DNA) from the filter membrane. The output from the elution module 4260 can be selectively placed in fluid communication with the filter assembly 4230, when the filter assembly is toggled into its second (or backflow) configuration, as described above. Thus, the elution module 4260 can include any suitable flow control devices, such as check valves, duck-bill valves, or the like to prevent flow back towards and/or into the elution volume.

Importantly, as described herein, the elution module 4260 is configured such that it cannot be actuated out of the desired sequence of operations. Specifically, the elution module 4260 is configured to be locked until after the biological sample has been conveyed to the sample preparation module 4200 and the wash operation (described above) has occurred. The elution module 4260 includes the elution portion 4104 of the sample transfer manifold 4100, the reagent (or third) actuator 4080, and an elution plunger 4270. Referring to FIGS. 14-18, and 21, the elution portion 4104 of the sample transfer manifold 4100 includes a cylindrical housing 4262 that defines an elution volume 4263 within which the elution buffer (or composition) is contained.

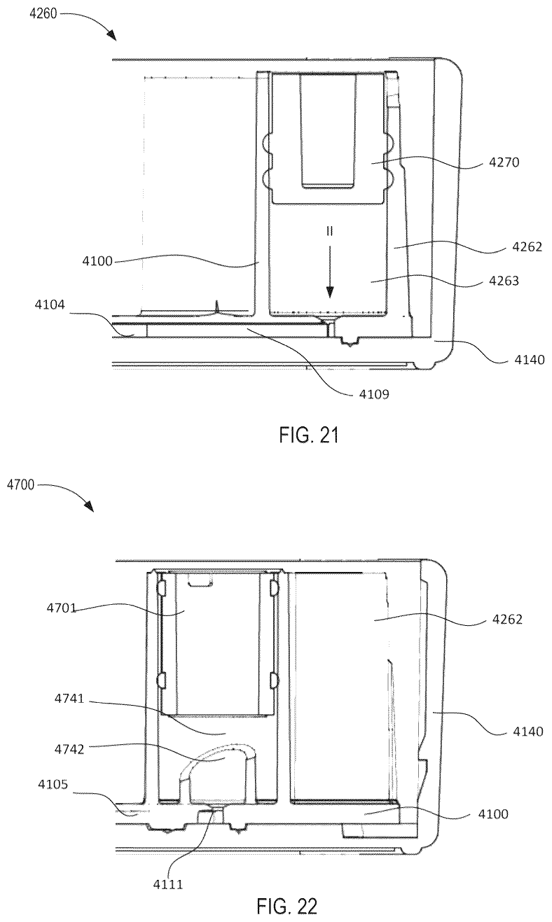

As shown in FIG. 21, the elution plunger 4270 is sealingly engaged with in inner side wall of the cylindrical housing 4262. The elution plunger 4270 can include any number of seals (e.g., O-rings, gaskets, elastomeric portions, or the like) to fluidically isolate the volume 4263 from the external volume of the device 4000. In some embodiments, the elution plunger can include a vent opening to allow controlled fluid communication of the volume 4263 with an external volume. The elution composition within the volume 4263 can be any suitable solution of the types described herein.

The lower portion of the cylindrical housing 4262 defines an opening into the elution input passage 4109. In use, when the elution plunger 4270 is moved downward (in the direction shown by the arrow II), the elution composition is moved from within the volume 4263 to the elution input passage 4109. The elution input passage 4109 extends to the filter assembly 4230, and is communication with the elution input port 4248 described above. In this manner, after the biological sample and the wash solution are conveyed from the sample input volume 4068 to the filter assembly 4230, an elution composition can be backflushed through the filter assembly 4230, as described above.

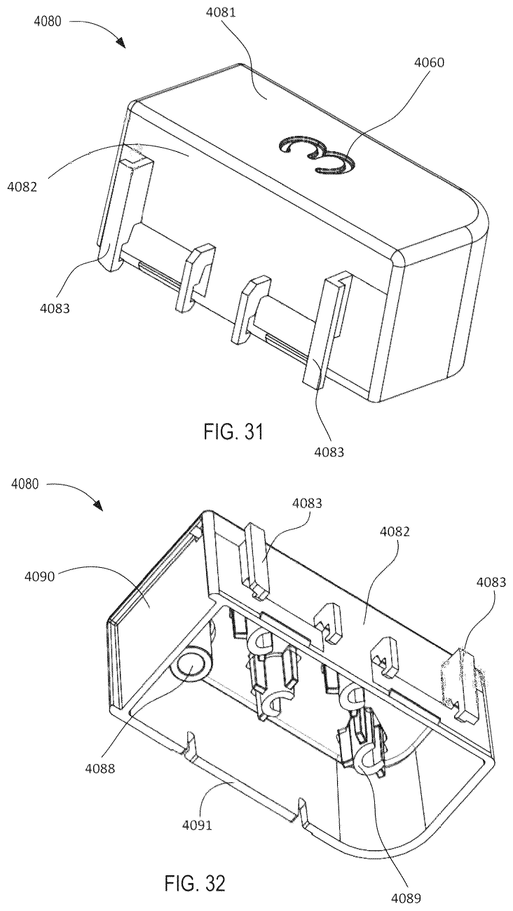

The elution module 4260 is actuated by the reagent (or third) actuator 4080. Referring to FIGS. 31-32, the reagent (or third) actuator 4080 includes a top surface 4081, a front surface 4091, a side surface 4090, a back surface 4082, and an inner surface. The top surface 4081 includes an indicator 4060 (i.e., “3”) to guide a user in the correct sequence of steps for use of the apparatus 4000. The side surface 4090 includes a recessed area that matingly engages with the side surface 4078 of the second actuator 4050. In this manner, when the third actuator 4080 is moved to its second position, the side surface 4078 of the second actuator 4070 guides the movement of the third actuator 4080. The front surface 4091 includes a protrusion that engages the side wall of the lower housing 4040 to lock and/or retain the third actuator 4080 in its second position. The inner surface includes a protrusion 4088 that engages the elution plunger 4270 and moves the elution plunger 4270, as described herein. The inner surface includes four reagent protrusions 4089 that each engage a reagent canister within the reagent module 4700 to puncture the reagent canisters when the third actuator 4080 is moved.

The back surface 4082 includes a pair of mounting protrusions 4083. The mounting protrusions 4083 are received within the actuator guide slots 4031. Additionally, the mounting protrusions 4083 each include a connection portion that is connected to the base 4096 of the spring clips 4095 (see FIG. 33) coupled to the third actuator 4080. In particular, two spring clips 4095 are coupled to the mounting protrusions 4083 such that when the third actuator 4080 is in its first position, the lock protrusion 4032 of the housing (i.e., between the two actuator guide slots 4031) is between the spring clips 4095 and the back surface 4082 (similar to the arrangement shown in FIG. 51). The resilient portion 4097 of each spring clip 4095 biases the lock portion 4098 of each spring clip 4095 against the lock protrusion 4032. Similar to the arrangement shown in FIG. 52, when the third actuator 4080 is moved to its second (or actuated) position, the lock portions 4098 becomes disengaged with the lock protrusion 4032 of the housing, and contact the back surface 4082 of the third actuator 4080. In this manner, the lock portions 4098 will engage the shoulder of the lock protrusion 4032 if the third actuator 4080 is moved back towards its first position. In this manner, the third actuator 4080 is retained in its second position.

As described above, the back surface 4083 also functions to actuate the filter assembly 4230 when the third actuator 4080 is moved to its second position. In some embodiments, the back surface 4083 can include specific protrusions and/or surfaces to ensure that the filter assembly 4230 is actuated at the proper time relative to the movement of the elution plunger 4270.

FIGS. 40-45 show various views of the reverse transcription module 4300 (also referred to as a lysing module or inactivation module). As described herein, the reverse transcription module provides suitable structure to rapidly heat the biological sample to perform any or all of the following: A) mixing the biological sample with desired reagents (e.g., a positive control reagent, a reverse transcriptase, a reducing agent, or any other reagents described herein), B) performing lysing operations to release target nucleic acids from the biological sample, C) performing a reverse transcription reaction to produce cDNA from the target RNA (e.g., for methods of detecting an RNA virus), and D) heating the resulting solution to inactivate the reverse transcriptase. The reverse transcription module 4300 includes a chamber body 4310, a bottom lid 4318, a heater 4330, and an electrode assembly. The chamber body 4310 and the bottom lid 4318 can be referred to as a flow member. The chamber body 4310 and the bottom lid 4318 define an input port 4312, a first (or holding) volume 4311, a vent 4314, a second (or inactivation) volume 4321, and an output port 4313. The input port 4312 can receive the eluent from the elution chamber and/or directly from the filter assembly 4230. In other embodiments, however, the input port 4312 can be fluidically coupled to a sample input module without the biological input being conveyed through a filter. In use, the eluent can flow into the reverse transcription module 4300 and be collected in the holding volume 4311. The sample can be lysed within the holding volume 4311. For example, in some embodiments, the eluent containing the target organisms can be heated by the heater 4330 to maintain the eluent at or above a target lysing temperature. Similarly stated, in some embodiments, the heater 4330 can be coupled to the chamber body 4310 and/or the bottom lid 4318 such that the heater 4330 can convey thermal energy into the reverse transcription module 4300 to produce a lysing temperature zone within the holding volume 4311. The lysing temperature zone can maintain the eluent at any of the temperatures and for any of the time periods described herein.

The vent opening 4314 is in fluid communication with the first volume 4311, and thus allows air to flow into or out of the reverse transcription module 4300 (including the first volume 4311 and the second volume 4321) as sample is conveyed into and/or out of the reverse transcription module 4300. The vent 4314 can also relieve pressure within either of the first volume 4311 or the second volume 4321 when the eluent is heated. Although shown as being a permanent vent (i.e., a vent having a fixed opening), in some embodiments, the reverse transcription module 4300 (or any of the reverse transcription modules described herein) can have an active vent. For example, in some embodiments, the reverse transcription module 4300 can include a valve that controls the venting of pressure and/or air from within the reverse transcription module 4300.

The first volume 4311 is in fluid communication with the second volume 4322. In this manner, the eluent (or biological sample) can flow from the first (or holding) volume 4311 through the second (or inactivation) volume 4321 of the reverse transcription module 4300. More specifically, when a pressure gradient is applied across the input port 4312 and the output port 4313, the eluent can flow from the holding volume 4311 (first volume) through the second volume 4322. The pressure gradient can be applied by any suitable mechanism, such as for example, a pump (e.g., the fluidic drive module 4400). As shown, the second volume 4321 is a serpentine channel that provides a high surface area to volume ratio. This arrangement allows for rapid lysis, reverse transcription, and/or inactivation of the lysis enzymes in the eluent. The eluent, after being flowed through the inactivation segment, may be flowed into the output port 4313 to be collected and/or conveyed to an amplification module (not shown).

As described above, the flow member is in contact with a heating element 4330, which can be, for example, a printed circuit board (PCB) heater. The heating element 4330 may function to heat the eluent as it flows through the second volume 4311 at a high temperature sufficient to perform a reverse transcription reaction (to produce cDNA) and/or inactivate the one or more lysis enzymes contained within the eluent. For example, the heating element may heat the eluent to about 57° C., about 58° C., about 59° C., about 60° C., about 61° C., about 62° C., about 63° C., about 64° C., about 65° C., about 66° C., about 67° C., about 68° C., about 69° C., about 70° C., about 71° C., about 72° C., about 73° C., about 74° C., about 75° C., about 76° C., about 77° C., about 78° C., about 79° C., about 80° C., about 81° C., about 82° C., about 83° C., about 84° C., about 85° C., about 86° C., about 87° C., about 88° C., about 89° C., about 90° C., about 91° C., about 92° C., about 93° C., about 94° C., about 95° C., about 96° C., about 97° C., about 98° C., about 99° C., about 100° C. or greater than 100° C. By heating the liquid eluent to a high temperature, the lysis enzymes as well as any other enzymes present can be deactivated. In some embodiments, the sample can be heated to about 95 C for about 4 minutes.

In some embodiments, the heater on the PCB 4330 is specifically designed to heat the serpentine portion of the reverse transcription module 4300 (i.e., the second volume 4321) while not heating the holding volume 4311. Because the lid 4318 of the reverse transcription module 4300 is thick, the heater surface may be heated well above the desired temperature of the fluid. Since the electrodes 4971, 4972 (described in more detail below) are thermally conductive and come into direct contact with the fluid, the fluid surrounding the electrodes 4971, 4972 will experience the same temperature as the heater surface, which may cause evaporation. To minimize the heating of the holding volume 4311, a slot (not shown) may be cut in the PCB 4330 to isolate the heater from the portion of the PCB adjacent and/or in contact with the holding volume 4311. For example, in some embodiments, the heater 4330 can include a series of slots and/or openings as described in U.S. patent application Ser. No. 15/494,145, entitled “Printed Circuit Board Heater for an Amplification Module,” which is incorporated herein by reference in its entirety.

In some embodiments, the reverse transcription module 4300 can determine whether there is liquid in the first volume 4311 and/or the second volume 4321. Specifically, the reverse transcription module 4300 includes electrical probes to determine electrical resistance of the fluid within the first volume. In some embodiments, the molecular diagnostic device (e.g., the device 1000) can include an electronic controller configured to determine when the user has actuated the elution module (e.g., by pressing the reagent actuator 4080 described above) by detecting the presence of liquid in the first volume 4311. In this manner, the introduction of liquid into the first volume 4311 can trigger the start of the device.

Specifically, the control system and/or the reverse transcription module 4300 includes two electrodes 4971, 4972 inside the first volume 4311. The electrodes 4971, 4972 are connected to circuitry (e.g., a controller, not shown) that detects a resistance change between the two electrodes 4971, 4972. Fluid may be reliably detected between the electrodes 4971, 4972 due to the high gain of the circuit, which may easily differentiate between an open circuit condition (no fluid) and a non-negligible resistance across the electrodes 4971, 4972 (fluid detected). Use of a sample matrix with high salt concentration increases the conductivity of the fluid, which may make the fluid easily detectable even with variation across samples. The electrodes 4971, 4972 and the circuitry (not shown) are designed to detect fluid without impacting the biological processes that take place in the device. For example, the electrodes 4971, 4972 are specifically chosen so as not inhibit PCR reactions. In some embodiments, the electrodes 4971, 4972 are gold plated.

Both DNA and cells have a net charge so they may migrate in the presence of an electric field. Because the resistance change between the electrodes 4971, 4972 is determined by measuring a change in electric potential, precautions may be taken to minimize the impact of this electromotive force. For example, once fluid is detected voltage may be removed from the electrodes 4971, 4972 and they may be electrically shorted together. This ensures there is no potential difference between the electrodes 4971, 4972 and the charged particles (DNA, cells, salts, etc.) will not bind to the electrodes, which would prevent them from entering the amplification module (not shown).

After the lysing, reverse transcription, and/or inactivation operations, the output from the reverse transcription module 4300 can be conveyed into the mixing module (also referred to as simply the mixing chamber) 4500, which mixes the output of reverse transcription module 4300 with the reagents to conduct a successful amplification reaction. Similarly stated, the mixing module 4500 is configured to reconstitute the reagent in a predetermined input volume, while ensuring even local concentrations of reagents in the entirety of the volume. In some embodiments, the mixing chamber module 4500 is configured to produce and/or convey a sufficient volume of liquid for the amplification module 4600 to provide sufficient volume output to the detection module 4800. The mixing module 4500 can be any suitable mixing module, such as those shown and described in International Patent Publication No. WO2016/109691, entitled “Devices and Methods for Molecular Diagnostic Testing,” which is incorporated herein by reference in its entirety.

The fluidic drive (or transfer) module 4400 can be a pump or series of pumps configured to produce a pressure differential and/or flow of the solutions within the diagnostic test device 4000. Similarly stated, the fluid transfer module 4400 is configured to generate fluid pressure, fluid flow and/or otherwise convey the biological sample and the reagents through the various modules of the device 4000. The fluid transfer module 4400 is configured to contact and/or receive the sample flow therein. Thus, in some embodiments, the device 4000 is specifically configured for a single-use to eliminate the likelihood that contamination of the fluid transfer module 4400 and/or the sample preparation module 4200 will become contaminated from previous runs, thereby negatively impacting the accuracy of the results. The fluid transfer module 4500 can be any suitable fluid transfer module, such as those shown and described in International Patent Publication No. WO2016/109691, entitled “Devices and Methods for Molecular Diagnostic Testing,” which is incorporated herein by reference in its entirety.

After being mixed within the mixing module 4500, the prepared sample is then conveyed to the amplification module 4600 (as shown by the arrow EE in FIG. 2). The amplification module 4600 includes a flow member 4610 and a heater 4630. The flow member 4610 can be any suitable flow member that defines a volume or a series of volumes within which the that prepared solution S3 can flow and/or be maintained to amplify the target nucleic acid molecules within the solution S3. The heater 4630 can be any suitable heater or group of heaters coupled to the flow member 4610 that can heat the prepared solution within the flow member 4610 to perform any of the amplification operations as described herein. For example, in some embodiments, the amplification module 4600 (or any of the amplification modules described herein) can be similar to the amplification modules shown and described in U.S. patent application Ser. No. 15/494,145, entitled “Printed Circuit Board Heater for an Amplification Module,” which is incorporated herein by reference in its entirety. In other embodiments, the amplification module 4600 (or any of the amplification modules described herein) can be similar to the amplification modules shown and described in International Patent Publication No. WO2016/109691, entitled “Devices and Methods for Molecular Diagnostic Testing,” which is incorporated herein by reference in its entirety.

In some embodiments, the flow member 4610 defines a single volume within which the prepared solution is maintained and heated to amplify the nucleic acid molecules within the prepared solution. In other embodiments, the flow member 4610 can define a “switchback” or serpentine flow path through which the prepared solution flows. Similarly stated, the flow member 4610 defines a flow path that is curved such that the flow path intersects the heater 4630 at multiple locations. In this manner, the amplification module 4600 can perform a “flow through” amplification reaction where the prepared solution flows through multiple different temperature regions.

The flow member 4610 (and any of the flow members described herein) can be constructed from any suitable material and can have any suitable dimensions to facilitate the desired amplification performance for the desired volume of sample. For example, in some embodiments, the amplification module 4600 (and any of the amplification modules described herein) can perform 4000X or greater amplification in a time of less than 45 minutes. For example, in some embodiments, the flow member 4610 (and any of the flow members described herein) is constructed from at least one of a cyclic olefin copolymer or a graphite-based material. Such materials facilitate the desired heat transfer properties into the flow path. Moreover, in some embodiments, the flow member 4610 (and any of the flow members described herein) can have a thickness of less than about 0.5 mm. In some embodiments, the flow member 4610 (and any of the flow members described herein) can have a volume about 450 microliters or greater, and the flow can be such that at least 40 microliters of sample is amplified. In other embodiments, at least 20 microliters of sample are amplified by the methods and devices described herein. In other embodiments, at least 30 microliters of sample are amplified by the methods and devices described herein. In yet other embodiments, at least 50 microliters of sample are amplified by the methods and devices described herein.

The heater 4630 can be any suitable heater or collection of heaters that can perform the functions described herein to amplify the prepared solution. In some embodiments, the heater 4630 can establish multiple temperature zones through which the prepared solution flows and/or can define a desired number of amplification cycles to ensure the desired test sensitivity (e.g., at least 30 cycles, at least 34 cycles, at least 36 cycles, at least 38 cycles, or at least 40 cycles). The heater 4630 (and any of the heaters described herein) can be of any suitable design. For example, in some embodiments, the heater 4630 can be a resistance heater, a thermoelectric device (e.g. a Peltier device), or the like. In some embodiments, the heater 4630 can be one or more linear “strip heaters” arranged such that the flow path crosses the heaters at multiple different points. In other embodiments, the heater 4630 can be one or more curved heaters having a geometry that corresponds to that of the flow member 4610 to produce multiple different temperature zones in the flow path.

Although the amplification module 4600 is generally described as performing a thermal cycling operation on the prepared solution, in other embodiment, the amplification module 4600 can perform any suitable thermal reaction to amplify nucleic acids within the solution. In some embodiments, the amplification module 4600 (and any of the amplification modules described herein) can perform any suitable type of isothermal amplification process, including, for example, Loop Mediated Isothermal Amplification (LAMP), Nucleic Acid Sequence Based Amplification (NASBA), which can be useful to detect target RNA molecules, Strand Displacement Amplification (SDA), Multiple Displacement Amplification (MDA), Ramification Amplification Method (RAM), or any other type of isothermal process

The detection methods enabled by the device 4000 include sequential delivery of the detection reagents and other substances within the device 4000. Further, the device 4000 is configured to be an “off-the-shelf” product for use in a point-of-care location (or other decentralized location), and is thus configured for long-term storage. Accordingly, the reagent storage module 4700 is configured for simple, non-empirical steps for the user to remove the reagents from their long-term storage containers, and for removing all the reagents from their storage containers using a single user action. In some embodiments, the reagent storage module 4700 and the rotary selection valve 4340 are configured for allowing the reagents to be used in the detection module 4800, one at a time, without user intervention.

Specifically, the device 4000 is configured such that the last step of the initial user operation (i.e., the depressing of the reagent actuator 4080) results in dispensing the stored reagents. This action crushes and/or opens the sealed reagent containers present in the assembly and relocates the liquid for delivery. The rotary venting selector valve 4340 allows the reagent module 4700 to be vented for this step, and thus allows for opening of the reagent containers, but closes the vents to the tanks once this process is concluded. Thus, the reagents remain in the reagent module 4700 until needed in the detection module 4800. When a desired reagent is needed, the rotary valve 4340 opens the appropriate vent path to the reagent module 4700, and the fluidic drive module 4400 applies vacuum to the output port of the reagent module 4700 (via the detection module 4800), thus conveying the reagents from the reagent module 4700. The reagent module 4700 and the valve 4340 can be similar to the reagent modules and valves shown and described in International Patent Publication No. WO2016/109691, entitled “Devices and Methods for Molecular Diagnostic Testing,” which is incorporated herein by reference in its entirety.

The detection module 4800 is configured to receive output from the amplification module 4600 and reagents from the reagent module 4700 to produce a colorimetric change to indicate presence or absence of target organism in the initial input sample. The detection module 4800 also produces a colorimetric signal to indicate the general correct operation of the test (positive control and negative control). In some embodiments, color change induced by the reaction is easy to read and binary, with no requirement to interpret shade or hue. The detection module 4800 can be similar to the detection modules shown and described in International Patent Publication No. WO2016/109691, entitled “Devices and Methods for Molecular Diagnostic Testing,” which is incorporated herein by reference in its entirety.

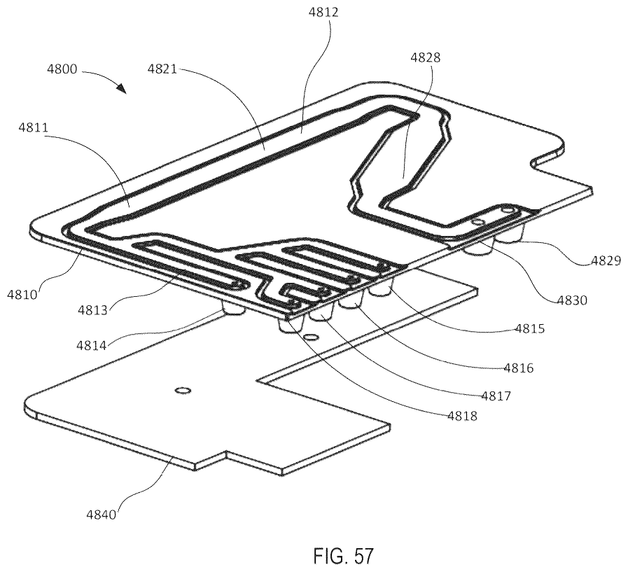

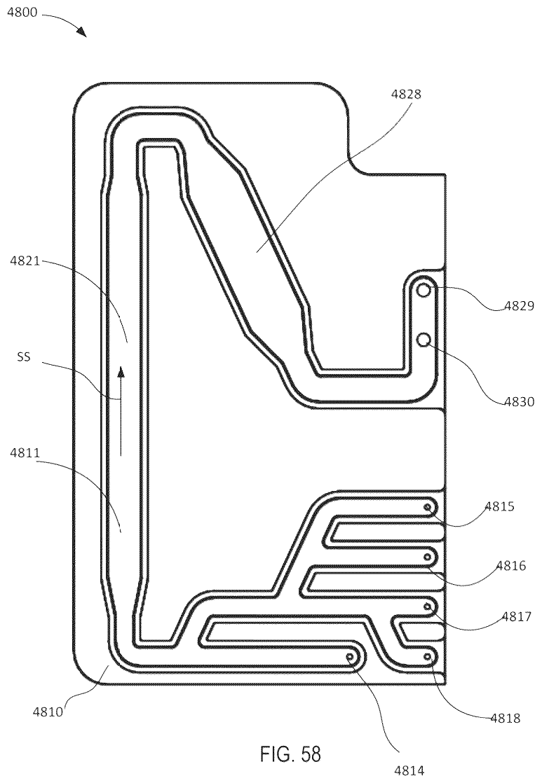

Referring to FIGS. 57 and 58, the detection module includes a lid (not shown, but similar to the lid 2802 shown and described above), a detection housing 4810 and a heater 4840. The heater 4840 can be similar to any of the circuit board heaters described herein and also shown and described in International Patent Publication No. WO2016/109691, entitled “Devices and Methods for Molecular Diagnostic Testing,” which is incorporated herein by reference in its entirety.

The lid and the detection housing 4810 form a flow cell for detection. The housing 4810 defines a detection chamber/channel 4812 having a sample inlet portion 4813, a reagent inlet portion, a detection portion 4821, and an outlet portion 4828. The sample inlet portion 4813 includes the sample inlet port 4814, which is fluidically coupled to the outlet of the amplification module 4600 and receives the amplified sample. The reagent inlet portion includes a first reagent inlet port 4815, a second reagent inlet port 4816, a third reagent inlet port 4817, and a fourth reagent inlet port 4818. The first reagent inlet port 4815 is coupled to the reagent module 4700 via the fluid passage 4043 of the vertical manifold 4035. Thus, in use a first reagent (e.g., a detection reagent) can be conveyed into the detection channel 4812 via the first reagent inlet port 4815. The second reagent inlet port 4816 is coupled to the reagent module 4700 via the fluid passage 4044 of the vertical manifold 4035. Thus, in use a second reagent (e.g., a wash solution) can be conveyed into the detection channel 4812 via the second reagent inlet port 4816. The third reagent inlet port 4817 is coupled to the reagent module 4700 via the fluid passage 4045 of the vertical manifold 4035. Thus, in use a third reagent (e.g., a detection reagent) can be conveyed into the detection channel 4812 via the third reagent inlet port 4817. The fourth reagent inlet port 4818 is coupled to the reagent module 4700 via the fluid passage 4046 of the vertical manifold 4035. Thus, in use a fourth reagent (e.g., a second flow of a detection reagent) can be conveyed into the detection channel 4812 via the first reagent inlet port 4818.

The detection channel 4812 includes an entrance portion 4811, a detection portion 4821, and outlet portion 4828. The detection portion (or “read lane”) 4821 is defined, at least in part by, and/or includes a series of detection surfaces. The detection surfaces 4821 include a series of capture probes to which the target amplicon can be bound when the detection solution flows across the detection surface 4821. The capture probes can be any suitable probes formulated to capture or bind to the target amplicon, such as those described above with respect to the detection module 2800. Specifically, the detection portion 4821 includes five detection surfaces. Each of the detection surfaces are chemically modified to contain a desired capture probe configuration. Specifically, a first detection surface can include a hybridization probe specific to a first target pathogen (e.g., Neisseria gonorrhea (NG) or flu A). A second detection surface can include a hybridization probe specific to a second target pathogen (e.g., Chlamydia trachomatis (CT) or flu B). A third detection surface can include a hybridization probe specific to a third target pathogen (e.g., Trichomonas vagina/is (TV) or COVID-19). A fourth detection surface can include non-target probe for a negative control. A fifth detection surface can include a hybridization probe for a positive control (A. fischeri, N. subflava, or the like).

The negative control surface 4825 includes a non-target probe and should always appear white (no color). In some embodiments, the negative control surface can be placed as the last spot (i.e., in the direction of flow as indicated by the arrow SS) because this arrangement shows whether the reagent volumes, fluidic movement, and wash steps were working properly.

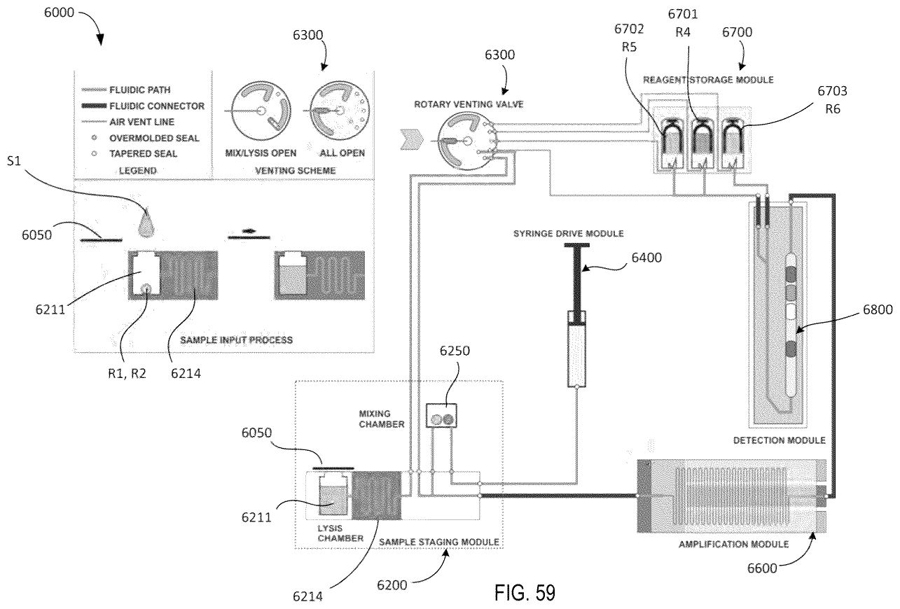

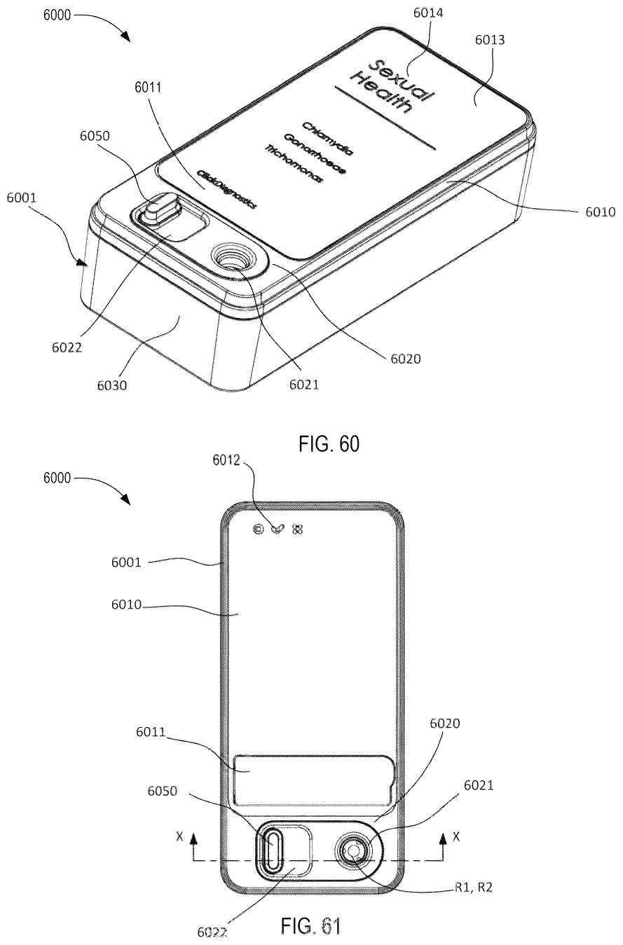

FIG. 59 is a schematic illustration of a molecular diagnostic test device 6000, according to an embodiment. The schematic illustration describes the primary components of the test device 6000 as shown in FIGS. 60-62. The test device 6000 is an integrated device (i.e., the modules are contained within a single housing) that is suitable for use within a point-of-care setting (e.g., doctor's office, pharmacy or the like), decentralized test facility, or at the user's home. In some embodiments, the device 6000 can have a size, shape and/or weight such that the device 6000 can be carried, held, used and/or manipulated in a user's hands (i.e., it can be a “handheld” device). A handheld device may have dimensions less than 15 cm×15 cm×15 cm, or less than 15 cm×15 cm×10 cm, or less than 12 cm×12 cm×6 cm. In other embodiments, the test device 6000 can be a self-contained, single-use device. Similarly stated, the test device 6000 is a stand-alone device that includes all necessary substances, mechanisms, and subassemblies to perform any of the molecular diagnostic tests described herein. As such, the device 6000 does not require any external instrument to manipulate the biological samples, and only requires a connection to a power source (e.g., a connection to an A/C power source, coupling to a battery, or the like) to complete the methods described herein. In some embodiments, the test device 6000 can be configured with lock-outs or other mechanisms to prevent re-use or attempts to re-use the device.

Further, in some embodiments, the device 6000 can be a CLIA-waived device and/or can operate in accordance with methods that are CLIA waived. Similarly stated, in some embodiments, the device 6000 (and any of the other devices shown and described herein) is configured to be operated in a sufficiently simple manner, and can produce results with sufficient accuracy to pose a limited likelihood of misuse and/or to pose a limited risk of harm if used improperly. In some embodiments, the device 6000 (and any of the other devices shown and described herein), can be operated by a user with minimal (or no) scientific training, in accordance with methods that require little judgment of the user, and/or in which certain operational steps are easily and/or automatically controlled. In some embodiments, the molecular diagnostic test device 6000 can be configured for long term storage in a manner that poses a limited likelihood of misuse (spoilage of the reagent(s), expiration of the reagents(s), leakage of the reagent(s), or the like). In some embodiments, the molecular diagnostic test device 6000 is configured to be stored for up to about 36 months, up to about 32 months, up to about 26 months, up to about 24 months, up to about 18 months, up to about 6 months, or any values there between.

The test device 6000 is configured to manipulate a biological sample S1 to produce one or more output signals associated with a target cell. Specifically, the device 6000 includes a sample preparation module 6200, a fluidic drive (or fluid transfer) module 6400, an amplification module 6600, a detection module 6800, a reagent module 6700, a valve 6300, and a control module (not shown).

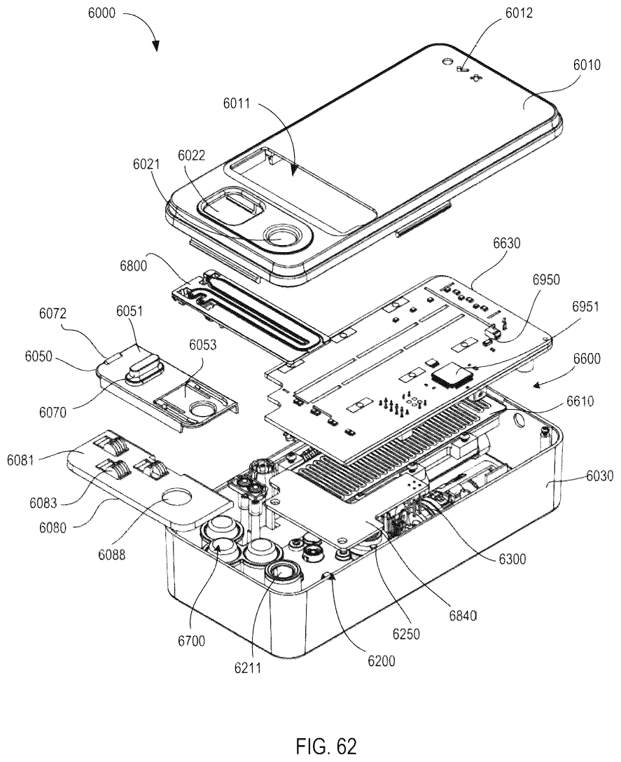

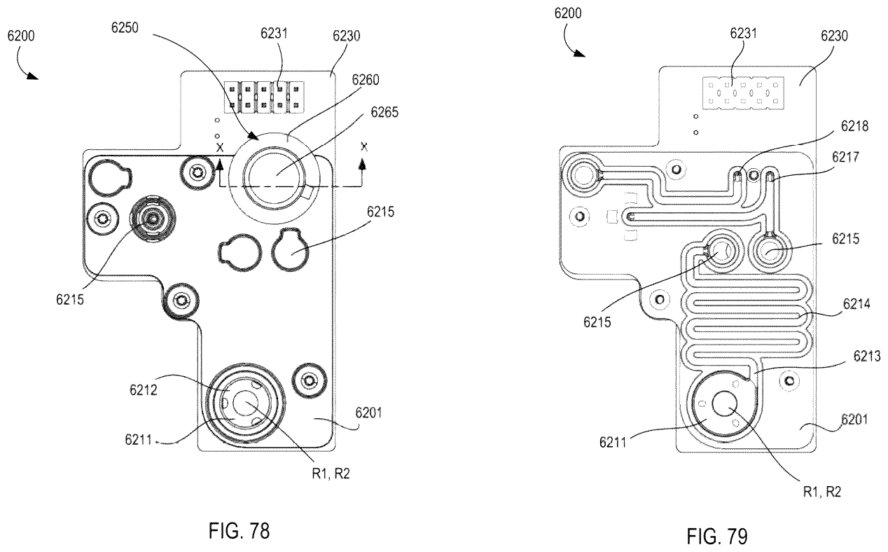

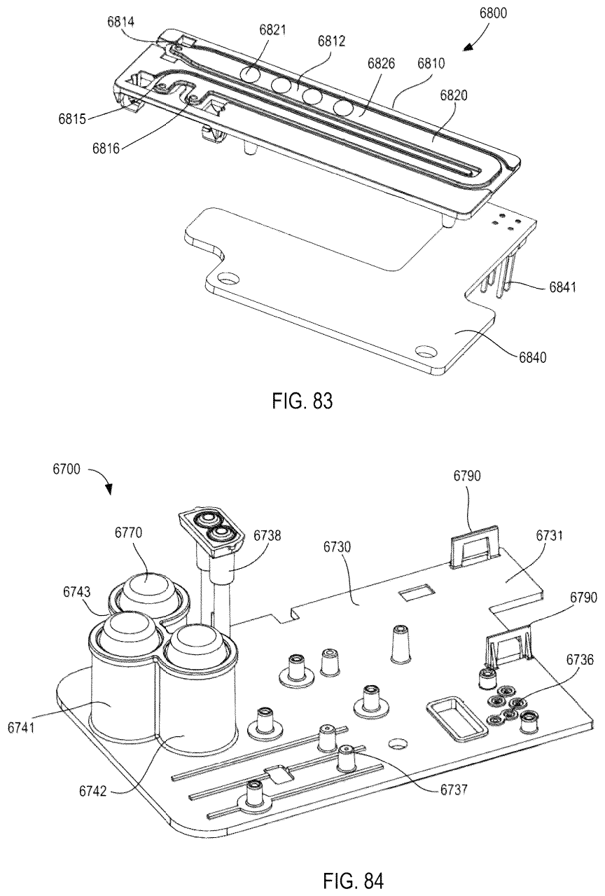

FIGS. 60-92 show various views of the molecular diagnostic test device 6000. The test device 6000 is configured to manipulate an input sample to produce one or more output signals associated with a target cell, according to any of the methods described herein. The diagnostic test device 6000 includes a housing 6001 (including a top portion 6010 and a bottom portion 6030), within which the modules described herein are fully or partially contained. Similarly stated, the housing 6001 (including the top portion 6010 and/or the bottom portion 6030) at least partially surround and/or enclose the modules. FIGS. 62-65 are various views that show the sample preparation module 6200, the fluidic drive (or fluid transfer) module 6400, the amplification module 6600, the detection module 6800, the reagent module 6700, the fluid transfer valve 6300, and the electronic control module 6900 situated within the housing 6001. A description of the housing assembly 6001 if followed by a description of each module and/or subsystem.

The housing assembly 6001 includes a top housing 6010, a bottom housing 6030, and a lid 6050 (which functions as a cover and an actuator). As shown, the top housing 6010 defines a detection opening (or window) 6011 and a series of status light openings 6012. The top housing 6010 also includes a sample input portion 6020 and a label 6013. The status light openings 6012 are aligned with one or more light output devices (e.g., LEDs) of the electronic control module 6950. In this manner, a light output produced by such status lights is visible through the status light openings 6012. Such light outputs can indicate, for example, whether the device 6000 is receiving power from the power source, whether an error has occurred (e.g., an error associated with insufficient sample volume or the like), and whether the test has been successfully completed.

The detection opening (or window) is aligned with the detection module 6800. In this manner, the signal produced by and/or on each detection surface of the detection module 6800 is visible through the detection opening 6011. In other embodiments, the top housing 6010 need not include a detection opening 6011. For example, in such embodiments, the signal produced by the detection module 6800 is not visible to the naked eye, but instead is read using another method. For example, in some embodiments, the reading can include using a secondary device, such a mobile computing device to scan or otherwise receive the signal OP1. In yet other embodiments, the reading the result can include indirectly reading a secondary signal that conveys the results associated with (or describing) the primary output from the detection module 6800.

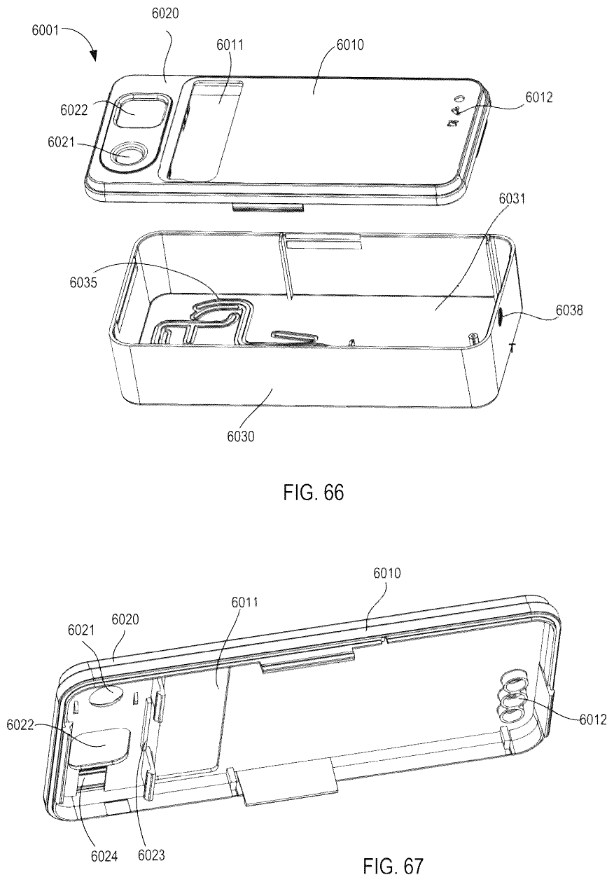

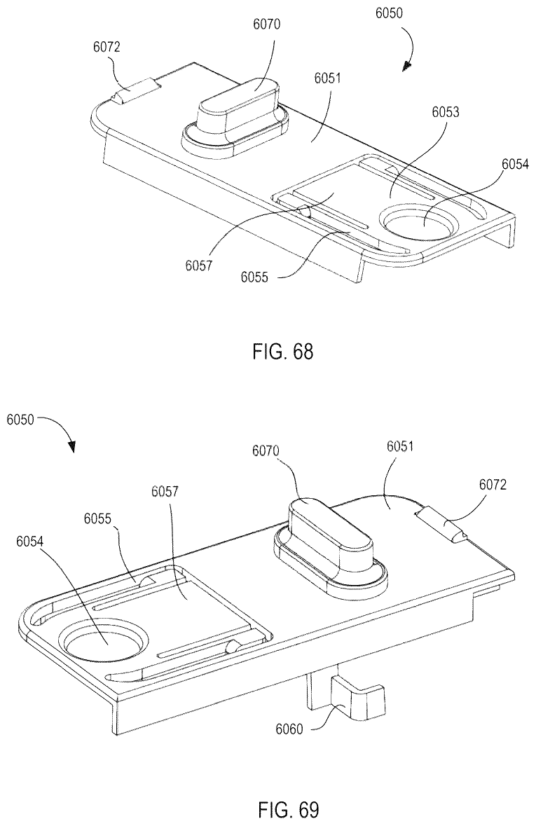

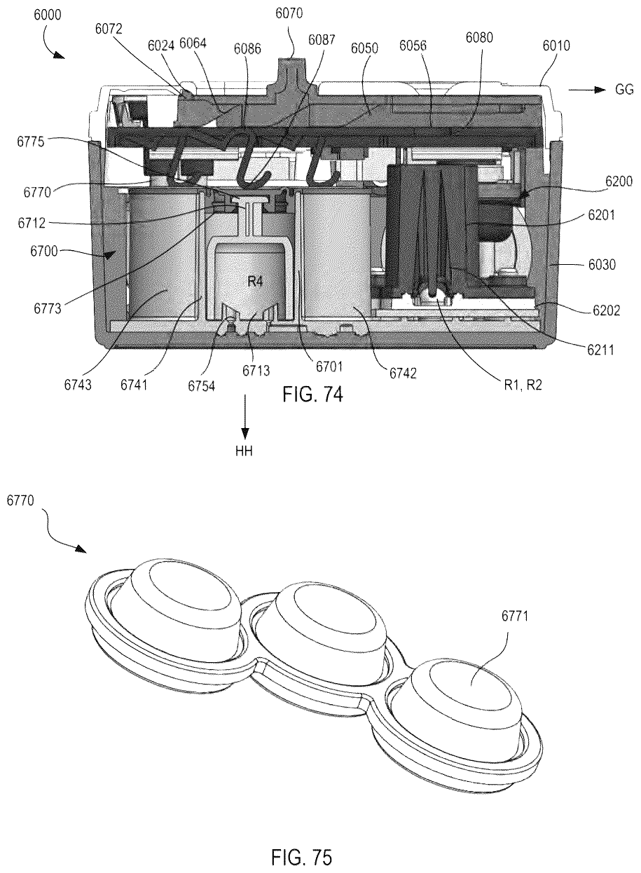

Referring to FIGS. 66 and 67, the sample input portion 6020 includes a set of guide rails 6023 and a lock recess 6024, both on the bottom (or inside) surface of the top housing 6010. The sample input portion 6020 also defines a sample input opening 6021 and an actuator opening 6022. The sample input opening 6021 is aligned with the input opening 6212 (of the sample preparation module 6200) and provides an opening through which a biological sample S1 can be conveyed into the device 6000. Additionally, the sample input portion also allows the lid (or actuator) 6050 to be movably coupled to the top housing 6010. Specifically, as shown in FIGS. 60, 73, and 74, the lid 6050 is coupled to the top housing 6010 such that the handle 6070 of the actuator extends through the actuator opening 6022. The actuator opening 6022 is elongated to allow for sliding movement of the lid 6050 relative to the top housing 6010, as described herein. Additionally, the guide rails 6023 are coupled to corresponding guide slots 6055 of the lid 6050 (see FIGS. 68 and 69) to facilitate the sliding movement of the lid 6050. As shown in FIGS. 73 and 74, the lock recess 6024 of the top housing 6010 is configured to receive the lock protrusion 6072 of the lid 6050 (see FIGS. 68 and 69) when the lid is in the second (or closed) position to prevent movement of the lid. In this manner, the top housing 6010 includes a lock mechanism that maintains the lid 6050 in its second (or closed) position to prevent reuse of the diagnostic device 6000, transfer of additional samples into the device 6000, or attempts to actuate the lid 6050 multiple times.

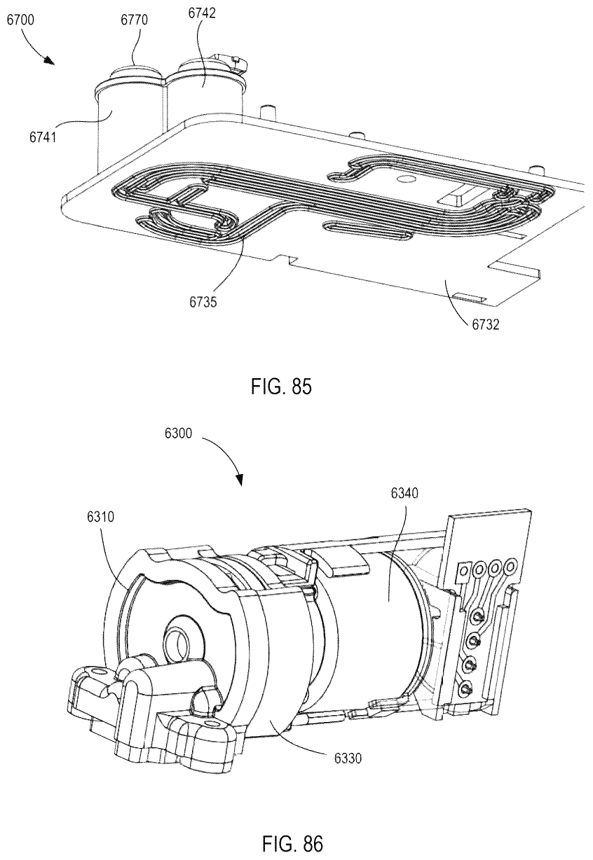

The lower housing 6030 includes a bottom plate 6031 and defines a volume within which the modules and or components of the device 6000 are disposed. As shown in FIG. 66, the bottom plate 6031 defines a series of flow channels 6035 that are aligned with flow channels of other components within the device to allow for fluid transfer between the various modules and components without the need for tubing, clamps, and the like. Specifically, as shown in FIG. 85, the bottom of the reagent module 6700 defines a series of flow channels 6735 that correspond to the flow channels 6035 in the bottom plate 6031 and thus facilitate transfer of fluids within the device. As shown in FIG. 66, the lower housing 6030 defines an opening 6038 that is aligned with a power input port of the electronic control module 6950. In use, an end of a power cord can be coupled to the electronic control module 6950 via the opening 6038.

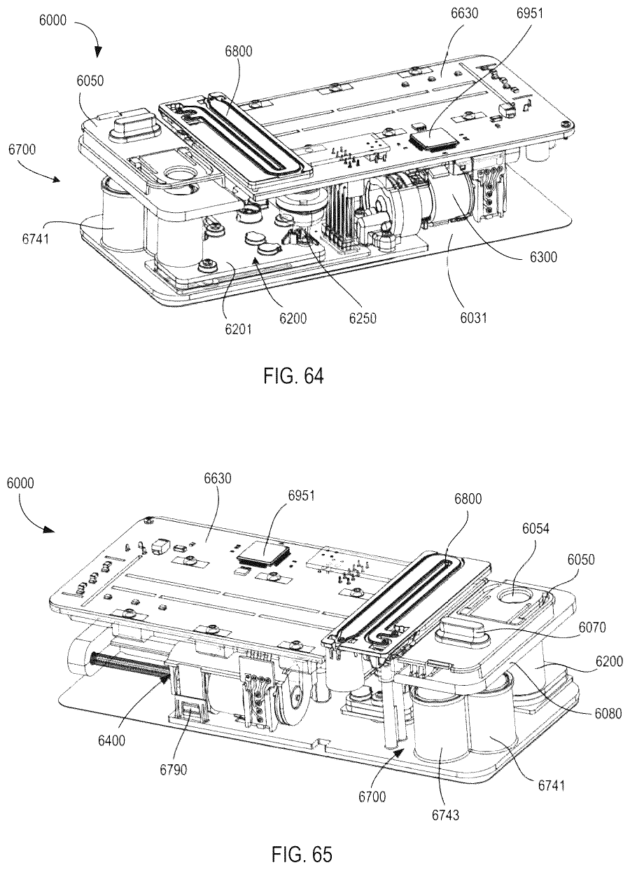

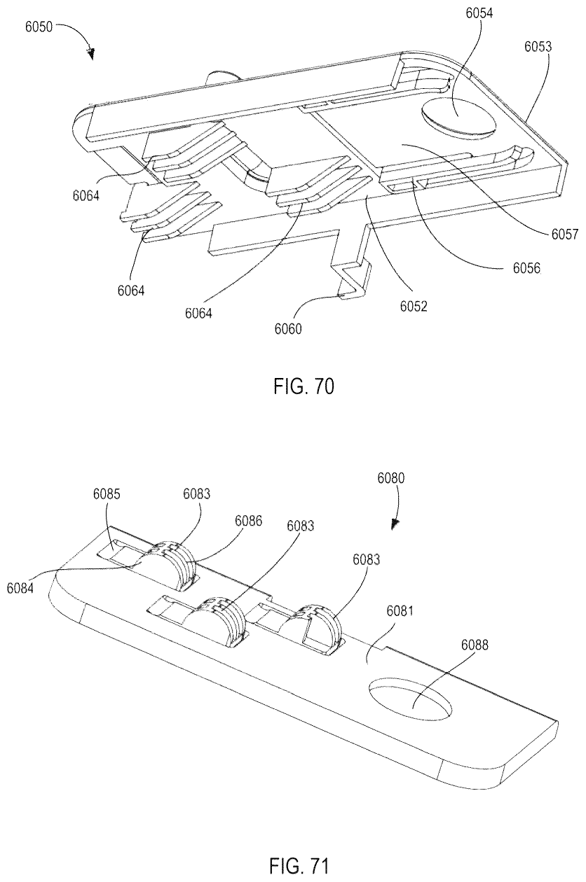

As shown in FIGS. 68-70, the lid 6050 includes a first (or outer) surface 6051 and a second (or inner) surface 6052. Referring to FIGS. 73 and 74, the lid 6050 is coupled to the housing 6001 and is positioned between the top housing 6010 and the flexible plate 6080. As described below, the lid 6050 and the flexible plate 6080 collectively actuate the reagent module 6700 when the lid 6050 is moved relative to the housing 6001. As shown in FIG. 70, the inner surface 6052 defines a pair of guide slots 6055 and includes a pair of guide rails 6056. As described above, the guide slots 6055 are coupled to corresponding guide rails 6023 of the housing 6001 to facilitate the sliding movement of the lid 6050. The guide rails 6056 of the lid 6050 are configured to engage with the flexible plate 6080, and thus also facilitate sliding movement of the lid 6050 (relative to the flexible plate 6080). As shown by the arrow GG in FIG. 64, the lid 6050 is configured to move relative to the housing 6001 from a first (or opened) position (FIG. 73) to a second (or closed) position (FIG. 74).

The lid 6050 is configured to perform a variety of functions when moved relative to the housing 6001, thereby facilitating actuation of the device 6000 via a single action. Specifically, the lid 6050 includes a seal portion 6053, a switch portion 6060, and three reagent actuators 6064. The seal portion 6053 (also referred to as a cover portion) includes a cover surface 6057 and defines an input opening 6054. When the lid 6050 is in the opened position (see e.g., FIGS. 60 and 61), the input opening 6054 is aligned with each of the sample input opening 6021 of the top housing 6010 and the input opening 6212 of the sample preparation module 6200 and thus provides an opening through which the biological sample S1 can be conveyed into the device 6000. The cover surface 6057 is a flat surface that covers (or obstructs each of the sample input opening 6021 of the top housing 6010 and the input opening 6212 when the lid is in the closed position. Specifically, the cover surface 6057 is spaced apart from the input opening 6212 and/or the sample input opening 6021 when the lid 6050 is in the opened position, but covers the input opening 6212 and/or the sample input opening 6021 when the lid 6050 is in the closed position. In some embodiments, the seal portion 6053 and/or the cover surface 6057 includes a seal, gasket, or other material to fluidically isolate the sample input volume 6211 (of the sample preparation module 6200) when the lid 6050 is in the closed position.

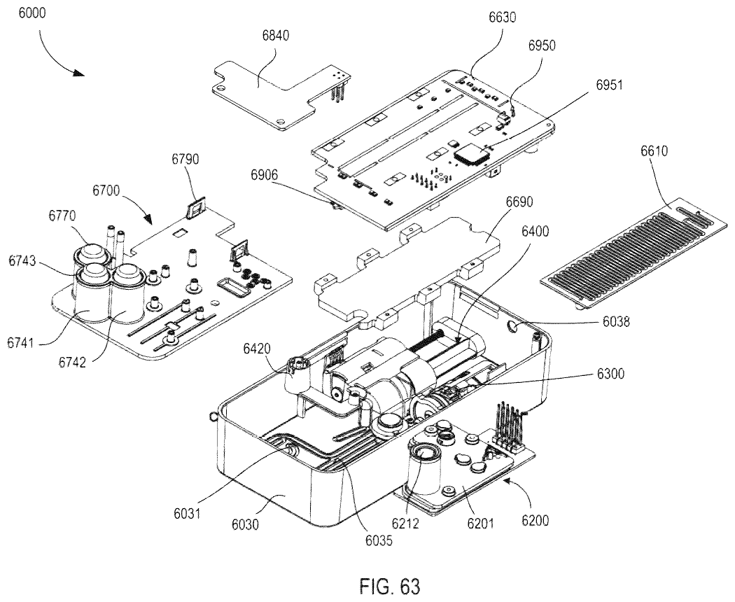

In addition to covering the input opening 6212, closing the lid 6050 also actuates other mechanisms within the device 6000. Specifically, as shown in FIGS. 69 and 70, the switch portion 6060 includes a protrusion that actuates the switch 6906 (FIG. 63) when the lid 6050 is moved from the opened position to the closed position. When the switch is actuated (i.e., is moved from a first state to a second state), power from the power source (e.g., the power source 6905) can be provided to the electronic control module 6950 and any other components within the device 6000 that require power for operation. For example, in some embodiments, power is provided to any of the heaters (e.g., the heater 6230 of the sample preparation module 6200, the heater 6630 of the amplification module 6600, and the heater 6840 of the detection module 6800) directly or via the electronic control module 6950. For example, this allows the heater 6230 to begin preheating for a lysis and/or reverse transcription operation after the lid 6050 is closed and the device 6050 is coupled to the power source 6905 without requiring further user action. Although the switch 6906 is shown as being a rocker switch that is actuated directly by the protrusion of the switch portion 6060, in other embodiments, the switch 6906 (and the corresponding switch portion 6060) can be any suitable switch that performs the functions described herein. For example, in some embodiments, the switch can be an isolation member that electrically isolates the power source 6905 from the remaining components of the electronic control module 6950. In such embodiments, the switch portion 6060 can be coupled to, and can remove, the isolation member (thereby electrically coupling the power source 6905 to the electronic control module 6950). In other embodiment, the switch portion 6060 is the isolation member, and no separate switch is included in the electronic control module 6950.

Referring to FIGS. 70 and 71, the reagent actuators 6064 include a series of ramped surfaces that exert an actuation force on a corresponding set of deformable actuators 6083 of the flexible plate 6080 when the lid 6050 is moved from the opened position (FIG. 73) to the closed position (FIG. 74). In this manner, the reagent actuators 6064 (and the deformable actuators 6083 of the flexible plate 6080) cause the reagent to be released from the sealed reagent containers within the reagent module 6700.

The outer surface 6051 of the lid 6050 includes a handle 6070 and a lock protrusion 6072. The handle 6070 extends through the actuator opening 6022 of the top housing 6010 and provides a structure that can be manipulated by the user to move the lid 6050 from the opened position to the closed position. The lock protrusion 6072 has a ramped (or angled) protrusion that is maintained in sliding contact with the inner surface of the top housing 6010 (see the inner surface shown in FIG. 67). Because the ramped surface of the lock protrusion 6072 forms an acute angle, the lock protrusion can be moved in the direction shown by the arrow GG in FIG. 74 to close the lid 6050. Additionally, the continuous contact between the lock protrusion 6072 and the top housing 6010 prevents inadvertent closure of the lid 6050 by providing some resistance (i.e., a friction force) to closing the lid. As shown in FIG. 74, when the lid 6050 is in the closed position, lock protrusion 6072 is received within the lock recess 6024 of the top housing 6010. The surface of the lock protrusion 6072 opposite the ramped surface forms a substantially 90-degree angle and thus prevents movement of the lid 6050 in the opposite direction when the lock protrusion 6072 is within the recess 6024. In this manner, the lid 6050 is irreversibly locked after being closed to prevent reuse of the device 6000 and/or the addition of supplemental sample fluids.

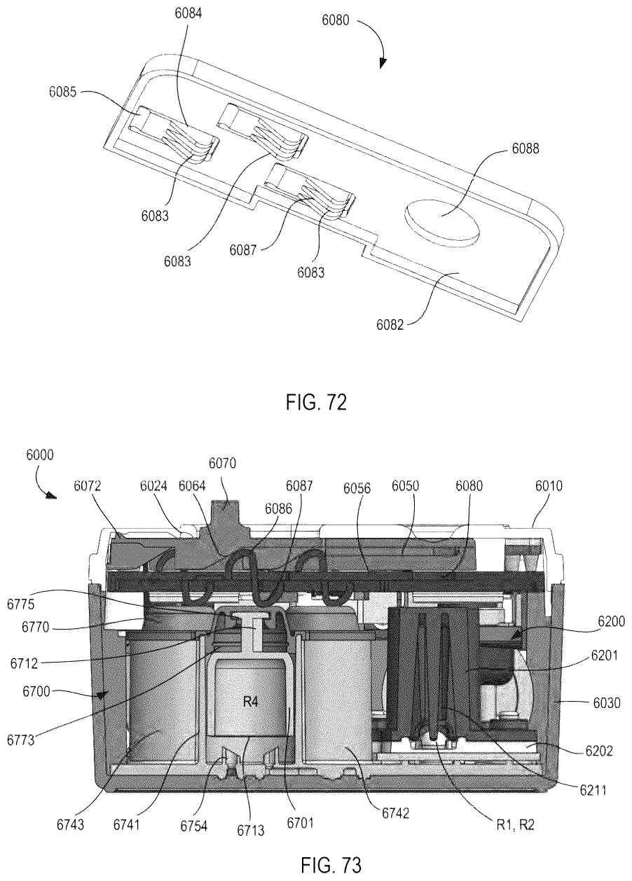

The flexible plate 6080 (shown in FIGS. 71 and 72) includes an outer surface 6081 and an inner surface 6082. As described above, the lid 6050 is movably disposed between the top housing 6010 and the flexible plate 6080. Similarly stated, the outer surface 6051 of the lid 6050 faces the inner surface of the top housing 6010 and the inner surface 6052 of the lid 6050 faces the outer surface 6081 of the flexible plate 6080. The flexible plate includes three deformable actuators 6083, each of which is aligned with a corresponding reagent actuator 6064 of the lid 6050 and one of the reagent containers 6701, 6702, 6703. Thus, when the lid 6050 is moved relative to the housing 6001, the reagent actuators 6064 and the deformable actuators 6083 actuate the reagent module 6700. In particular, as described in detail below, the reagent actuators 6064 and the deformable actuators 6083 move the reagent containers 6701, 6702, 6703 within the reagent manifold 6730 to release the reagents that are sealed within the containers.

The flexible plate 6080 defines a channel 6084 for the surrounds at least three sides of each of the deformable actuators 6083. Thus, each of the deformable actuators 6083 remains coupled to the flexible plate 6080 by a small strip of material (or living hinge) 6085. Accordingly, when the reagent actuator 6064 exerts an inward force on the outer surface 6086 of deformable actuator 6083, the deformable actuator bends or deforms inwardly towards the reagent module 6700 as shown by the arrow HH in FIG. 74. This action causes the inner surface 6087 of each of the deformable actuators 6083 to apply an inward force on the reagent containers (and the deformable support member 6770 thereby moving the reagent containers downward within the reagent manifold 6730, as shown by the arrow HH in FIG. 34.

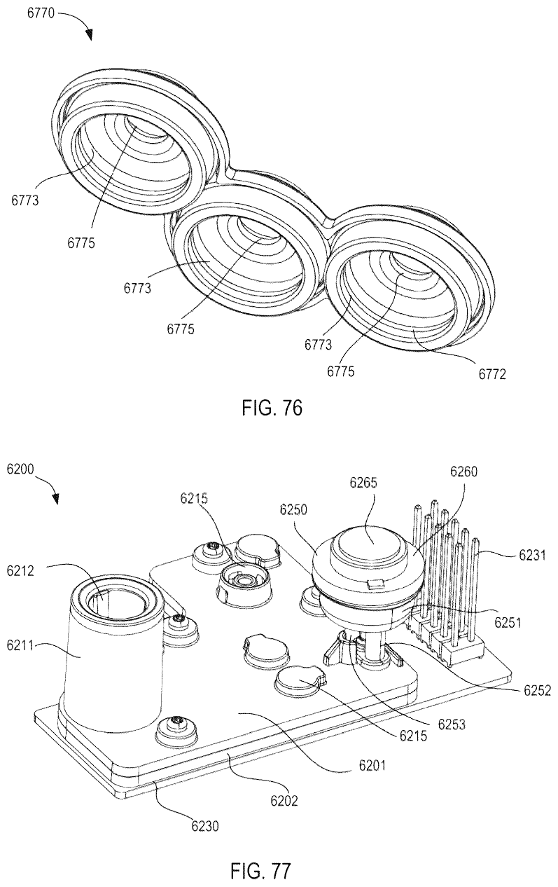

Referring to FIGS. 73, 74, 84, and 85, the reagent module 6700 includes a reagent manifold (or housing) 6730, three reagent containers 6701, 6702, 6703, and a deformable support member 6770 (see FIGS. 75 and 76). The reagent module 6700 provides mechanisms for long-term storage of reagents within the sealed reagent containers, actuation of the reagent containers to release the reagents from the reagent containers for use during the methods described herein. In addition to providing storage and actuating functions, the reagent module 6700 also provides fluid interconnections to allow the reagents and/or other fluids to be conveyed within the device 6000. Specifically, as described herein, the reagent module 6700 is fluidically coupled to the fluid transfer valve 6300 in a manner that allows selective venting, fluid coupling, and/or conveyance of the reagents and substances within the device 6000.

The reagent module 6700 stores packaged reagents, identified herein as reagent R4 (a dual-purpose blocking and wash solution), reagent R5 (an enzyme reagent), and reagent R6 (a substrate), and allows for easy un-packaging and use of these reagents in the detection module 6800. As shown schematically in FIG. 59, the reagent module 6700 includes a first reagent container 6701 (containing the reagent R4), a second reagent container 6702 (containing the reagent R5), and a third reagent container 6703 (containing the reagent R6). Each of the reagent containers includes a connector at a first end portion and a frangible seal at a second, opposite end portion. Specifically, as shown in FIGS. 73 and 74, the first reagent container 6701 includes a connector 6712 and a frangible seal 6713. The connector 6712 connects the first reagent container 6701 to the mating coupling portion 6775 of the deformable support member 6770. The frangible seal 6713 is any suitable seal, such as, for example, a heat-sealed BOPP film (or any other suitable thermoplastic film). When the reagent container is pushed into the puncturers, the frangible seal breaks, allowing the liquid reagent to flow into the appropriate reagent reservoir when vented by the fluid transfer valve 6300. Although only the details of the first reagent container 6701 are shown and described herein, the second reagent container 6702 and the third reagent container 6703 have similar structure and function.

Referring to FIGS. 84 and 85, the reagent manifold 6730 includes a top (or outer) surface 6731 and a bottom (or inner) surface 6732. The reagent manifold 6730 includes three reagent tanks extending from the top surface 6731 and within which the reagent containers are disposed. Specifically, the reagent manifold includes a first reagent tank 6741 within which the first reagent container 6701 is disposed, a second reagent tank 6742 within which the second reagent container 6702 is disposed, and a third reagent tank 6743 within which the third reagent container 6703 is disposed. The reagent housing 6730 includes a pair of puncturers in the bottom portion of each reagent tank. The puncturers are configured to pierce the frangible seal of the respective reagent container when the reagent container is moved downward within the reagent housing 6730. Similarly stated, the reagent housing 6730 includes a set of puncturers that pierce a corresponding frangible seal to open a corresponding reagent container when the reagent module 6700 is actuated. Referring to FIGS. 73 and 74 as an example, the reagent housing 6730 includes a set of puncturers 6754 within the first reagent tank 6741. The reagent housing 6730 includes similar puncturers in the second reagent tank 6742 and the third reagent tank 6743. Further, the puncturers define a flow path that places the internal volume of the reagent container and/or the reagent tank in fluid communication with an outlet port of the reagent module 6700 after the frangible seal is punctured.

The deformable support member 6770 includes an outer surface 6771 and an inner surface 6772. As described above, the outer surface 6771 includes actuation regions that are aligned with one of the deformable actuators 6083 of the flexible plate 6080. The inner surface 6772 includes three seal portions 6773 and three coupling portions 6775. As shown in FIGS. 73 and 74, each of the seal portion 6773 is coupled to the reagent housing 6730 to fluidically isolate the internal volume (i.e., the reagent reservoir) of the corresponding reagent tank. The coupling portions 6775 are each coupled to one of the connectors of the corresponding reagent container. As an example, one of the seal portions 6773 is coupled to the top portion of the first reagent tank 6741 to fluidically isolate (or seal) the internal volume of the first reagent tank 6741. Additionally, one of the coupling portions 6775 is coupled to the connector 6712 of the first reagent container 6701.

The deformable support member 6770 is configured to deform from a first configuration (FIG. 73) to a second configuration (FIG. 74) in response to an actuation force exerted thereon (e.g., by the deformable actuator 6083). Moreover, the deformable support member 6770 is biased in the first (or undeformed) configuration. In this manner, the deformable support member 6770 supports each of the reagent containers in a “storage state” when the deformable support member 6770 is in the first configuration. Similarly stated, the deformable support member 6770 maintains the puncturer 6754 spaced apart from the frangible seal 6713 of the reagent container 6701 when the deformable support member is in the first configuration.

When the lid 6050 is moved, the downward force exerted by the deformable actuators 6083 cause the deformable support member 6770 to transition to the second (or deformed) configuration (FIG. 74). Similarly stated, when the downward force is sufficient to overcome the opposite, biasing force of the deformable support member 6770, the deformable support member 6770 is transitioned to the second configuration, as shown by the arrow HH in FIG. 74. This causes each of the reagent containers to move downward within the corresponding reagent tank, bringing the puncturers into contact with the frangible seal of each reagent container. Similarly stated, when the deformable support member 6770 is in the second configuration, the puncturers 6754 pierce the frangible seal 6713 of the reagent container 6701, thereby release the reagent R4 from within the reagent container 6701. Although FIG. 74 shows the actuation for only the first reagent container 6701, when the reagent module 6700 is actuated, each of the first reagent container 6701, the second reagent container 6702, and the third reagent container 6703 are actuated in this manner. Thus, in addition to covering the sample input opening and providing power to the electronic control module 6950, closing the lid 6050 also actuates all of the reagent containers.

Referring to FIG. 84, the outer surface 6731 of the reagent manifold 6730 includes a set of valve fluid interconnects 6736, a set of mixing chamber fluid interconnects 6737, and a set of detection module fluid interconnects 6738. Each of these fluid interconnects is coupled to one of the reagent tanks and/or other components within the device 6000 by the flow channels 6735 defined in the inner surface 6732. Additionally, the outer surface 6731 includes multiple mounting clips 6790. Thus, the valve fluid interconnects 6736 (and the appropriate channels 6735) provide fluidic coupling to the fluid transfer valve 6300, which is coupled to the top surface 6731 by one of the clips 6790. The mixing chamber fluid interconnects 6737 (and the appropriate channels 6735) provide fluidic coupling to the mixing assembly 6250, which is coupled to the top surface 6731. The detection module fluid interconnects 6738 (and the appropriate channels 6735) provide fluidic coupling to the detection module 6800.

FIGS. 77-81 show various views of the sample preparation module 6200. As described herein, the sample preparation (or reverse transcription) module 6200 can perform any or all of A) receiving the biological sample S1, B) mixing the biological sample with desired reagents (e.g., a positive control reagent R1 and a reverse transcriptase R2), C) performing lysing operations to release target RNA from the biological sample S1, D) performing a reverse transcription reaction to produce cDNA, and E) heating the resulting solution to inactivate the reverse transcriptase. Thus, in some embodiments, the sample preparation module enables an efficient, fast RT-PCR to be performed within a single environment or module. By eliminating the need for external sample preparation and a cumbersome instrument, the device 6000 is suitable for use within a point-of-care setting (e.g., doctor's office, pharmacy or the like), a decentralized location, or at the user's home and can receive any suitable biological sample S1. The biological sample S1 (and any of the input samples described herein) can be, for example, blood, urine, male urethral specimens, vaginal specimens, cervical swab specimens, and/or nasal swab specimens gathered using a commercially available sample collection kit. In some embodiments, the biological sample S1 can be a combined (or pooled) sample that includes multiple individual samples (e.g., from multiple different patients).

The sample preparation module 6200 includes a top body 6201, a bottom body 6202, a heater 6230, and a mixing assembly 6250. The top body 6201 and the bottom body 6202 can be referred to collectively as a sample preparation housing, a flow member or a reverse transcription chamber. Although the flow member is shown as being constructed from two pieces (the top body 6201 and the bottom body 6202) that are coupled together, in other embodiments, the flow member can be monolithically constructed. The sample preparation housing (i.e., the top body 6201 and the bottom body 6202) define a sample input opening 6212, a first (or holding) volume 6211, and a serpentine flow channel 6214. In some embodiments, the top body 6201 and/or the bottom body 6202 can define one or more vents. Such vents can allow air to flow into or out of the sample preparation module 6200 (including the first volume 6211 and the serpentine flow channel 6214) as sample is conveyed into and/or out of the sample preparation module 6200. Additionally, the top body 6201 includes a set of fluid interconnects 6215 that allow for fluidic coupling of the sample preparation module 6200 to the fluid transfer valve 6300 and other components within the device 6000.

The sample input opening 6212 is an opening through which the first (or holding) volume 6211 can be accessed. As described above, when the lid 6050 is in the opened position, the biological sample S1 can be conveyed into the holding volume 6211 via the sample input opening 6212. The first (or holding) volume 6211 is a volume within which the biological sample S1 can be mixed with reagents and also optionally heated. For example, in some embodiments the biological sample S1 can be collected in the holding volume 6211 and mixed with either or both of a control organism (identified as reagent R1) and a reverse transcriptase (identified as reagent R2). The control organism and the reverse transcriptase can each be lyophilized or otherwise in solid form. In some embodiments, the holding volume 6211 can include any of the reducing agents described herein. For example, in some embodiments, the lyophilized reagent 4190 can be lithium, sodium, potassium, aluminum amalgam, lithium aluminum hydride, sodium borohydride, sodium cyanoborohydride, lithium tri-butoxy aluminum hydride, dibutoxy aluminum hydride, lithium triethyl borohydride, tris(2-carboxyethyl)phosphine (TCEP), dithiothreitol (DTT), mercaptoethanol, cysteine, thioglycerol, thioglycolic acid, cysteamine, glutathione, N-acetylcysteine (NAC), β-mercaptoethanol, sodium dodecyl sulfate (SDS), sodium sulfite, ammonium sulfamate, sodium bisulfite, dithionite metabisulfite sulfur dioxide, vitamin C, ascorbic acid, and dimethylsulfoxide (DMSO). In some embodiments, the lyophilized reagent can be N-acetylcysteine (NAC). In some embodiments, the N-acetylcysteine (NAC) is N-acetyl-L-cysteine. In some embodiments, the N-acetylcysteine is N-acetyl-D-cysteine. In some embodiments, the reagent R1 or R2 can be a lyophilized pellet that includes the reducing agent. Including the reducing agent and/or other reagents in a solid form enclosed within the device 6000 facilitates long term storage (some reducing agents can lack long-term stability of stored in liquid form within universal transport media (UTM) or a dilution buffer. This arrangement also limits the likelihood of tampering or operator error associated with using the correct amount of reagent. Moreover, the reagents R1 and R2 can be secured within the holding volume 6211 to prevent the reagents R1 and R2 from inadvertently falling out of the device 6000, for example during storage, transportation, or use. For example, in some embodiments, the reagents can be secured within the holding volume 6211 by a cover, basket, or other structure within the holding volume 6211. In other embodiments, the lyophilized reagent is applied via a dry reagent coating process to a cover, basket, or other structure within the holding volume 6211.

In some embodiments, the reagent R1 is a positive control organism, such as Aliivibrio fischeri, N. subflava, or any other suitable organism. Specifically, Aliivibrio fischeri is suitable because it is gram negative, nonpathogenic, bio safety level 1, not harmful to the environment, and is extremely unlikely to be found on a human. The positive control surface within the detection module contains capture probes for both the control organism (e.g., A. fischeri) as well as each of the target organisms. This arrangement ensures that the positive control surface always produces color if the device functions correctly. In some embodiments, the reagent R2 contains the reverse transcriptase enzymes and other constituents to facilitate the RT-PCR methods described herein. For example, in some embodiments, the reagent R2 includes the salts needed to create the correct buffering environment for the RT-PCR. The reagent R2 is formulated to dissolve in the biological sample within the holding volume 6211.

The biological sample can be heated within the holding volume 6311 to lyse the cells within the biological sample S1 and further lyse (or release) the target RNA from any viruses contained with the biological sample S1. In other words, the biological sample S1 can be heated to both break apart the cells and also disrupt the viruses there to release target RNA for detection. Specifically, the heater 6230 is coupled to the sample preparation housing and/or the bottom body 6202 such that a first portion of the heater 6230 can convey thermal energy into the holding volume 6211. The first portion of the heater 6230 can maintain the biological sample S1 at any suitable temperature and for any of the time periods described herein. For example, in some embodiments, the biological solution can be maintained at a temperature within a lysing temperature range to release a ribonucleic acid (RNA) molecule. The lysing temperature range can be, for example, between about 25 C and about 70 C. In other embodiments, the lysing temperature range can be between about 25 C and about 50 C.

Referring to FIG. 79, which shows a top view cross-section of the sample preparation housing, the first volume 6211 is in fluid communication with the serpentine flow channel 6214, via the inlet opening 6213. In this manner, the lysed biological sample that is mixed with the RT enzyme (also referred to as a reverse transcription solution) can flow from the first (or holding) volume 6211 through the serpentine flow channel 6214. More specifically, when a pressure gradient is applied across the inlet opening 6213 and the output opening 6215 (e.g., via the fluidic drive module 6400), the reverse transcription solution can flow from the holding volume 6211 (first volume) through the serpentine flow channel 6214. The serpentine channel provides a high surface area to volume ratio, and thus allows for rapid RT-PCR and inactivation of the lysis and/or RT enzymes in the solution.

In use, the reverse transcription solution can be heated as it flows through the serpentine flow channel 6214 to perform RT-PCR and, in some embodiments, to further inactivate the enzymes. Specifically, the heater 6230 is coupled to the sample preparation housing and/or the bottom body 6202 such that a second portion of the heater 6230 can convey thermal energy into the serpentine flow channel 6214. The second portion of the heater 6230 can maintain the reverse transcription solution at any suitable temperature and for any of the time periods described herein. For example, in some embodiments, the reverse transcription solution can be maintained at a temperature within a reverse transcription temperature range to produce complementary deoxyribonucleic acid (cDNA) molecules. By rapidly progressing to the reverse transcription, the dwell time during which released RNA are present in the reverse transcription solution can be minimized. Reducing the dwell time can reduce the likelihood that the released RNA will be degraded by ribonuclease (RNase). Limiting such potential degradation by performing the lysing and RT-PCR in a single environment can reduce inconsistencies due to variation in the RNA degradation. Further, the rapid and single-environment methods enabled by the sample preparation module 6200 can allow the RT-PCR methods described herein to be completed without the use of a ribonuclease inhibitor and/or on an unfiltered sample. The reverse transcription temperature range can be, for example, between about 30 C and about 80 C. In other embodiments, the reverse transcription temperature range can be between about 50 C and about 60 C.

In addition to enabling a rapid RT-PCR, the sample preparation module 6200 can also heat the reverse transcription solution to a temperature sufficient to inactivate the one or more lysis or RT enzymes contained therein. For example, the heating element may heat the reverse transcription solution within the channel 6214 to about 57° C., about 58° C., about 59° C., about 60° C., about 61° C., about 62° C., about 63° C., about 64° C., about 65° C., about 66° C., about 67° C., about 68° C., about 69° C., about 70° C., about 71° C., about 72° C., about 73° C., about 74° C., about 75° C., about 76° C., about 77° C., about 78° C., about 79° C., about 80° C., about 81° C., about 82° C., about 83° C., about 84° C., about 85° C., about 86° C., about 87° C., about 88° C., about 89° C., about 90° C., about 91° C., about 92° C., about 93° C., about 94° C., about 95° C., about 96° C., about 97° C., about 98° C., about 99° C., about 100° C. or greater than 100° C. By heating the reverse transcription solution to a high temperature, the enzymes can be deactivated. In some embodiments, the sample can be heated to about 95 C for about 4 minutes.

As described above, the flow member is in contact with a heating element 6230, which can be, for example, a printed circuit board (PCB) heater. The heating element 6230 includes connectors 6231 and multiple, segmented portions, and thus can independently produce thermal energy into the holding volume 6211 and the serpentine flow channel 6214. In some embodiments, the heating element 6230 is designed to heat the serpentine portion 6214 of the sample preparation module 6200 while not heating the holding volume 6211, and vice-versa.

The reverse transcription solution, after being flowed through the reverse transcription process (and optionally the inactivation process), may be flowed via the output port 6215 through the fluid control valve 6300 and into the inlet port 6217 of the mixing assembly 6250. The mixing assembly 6250 mixes the output from the serpentine flow channel 6214 with the reagents (identified as R3) to conduct a successful amplification reaction. Similarly stated, the mixing module 6250 is configured to reconstitute the reagent R3 in a predetermined input volume, while ensuring even local concentrations of reagents R3 in the entirety of the volume. In some embodiments, the mixing assembly 6250 is configured to produce and/or convey a sufficient volume of liquid for the amplification module 6600 to provide sufficient volume output to the detection module 6800.

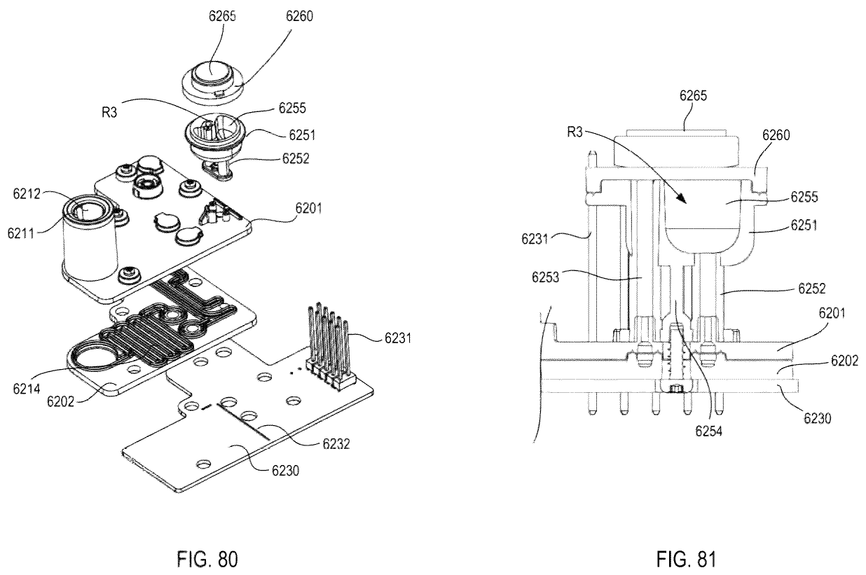

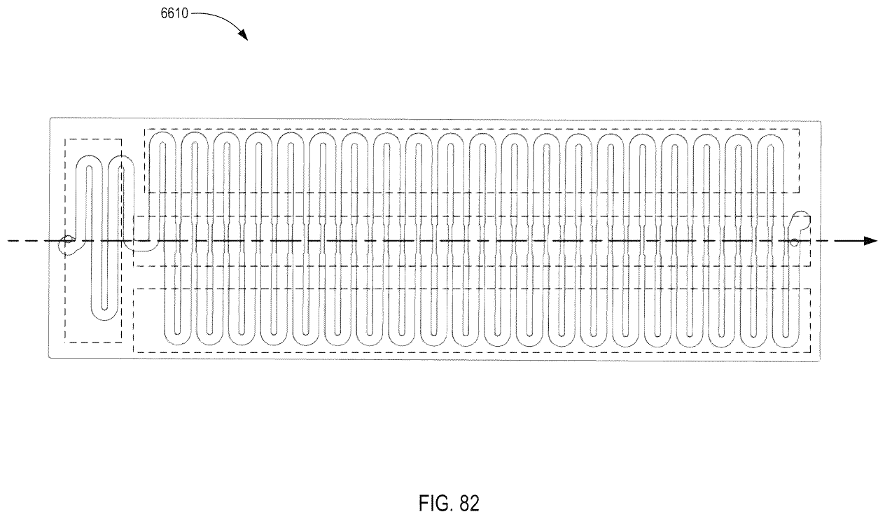

Referring to FIGS. 80 and 81, the mixing assembly 6250 is coupled to the top body 6201 and includes a bottom housing 6251, a top housing 6260, and a vibration motor 6265. The bottom housing 6251 defines a mixing reservoir 6255 and contains the amplification reagents R3 therein. The bottom housing 6251 includes an inlet coupling 6252 and an outlet coupling 6253, and is coupled to the top body 6201 by a support member 6254. The top housing 6260 encloses the mixing reservoir 6255 and provides a surface to which the vibration motor 6265 is mounted. The inlet coupling 6252, the outlet coupling 6253, and the support member 6254 can be constructed from any suitable material and can have any suitable size. For example, in some embodiments, the inlet coupling 6252, the outlet coupling 6253, and the support member 6254 are constructed to limit the amount of vibration energy from the motor 6265 that is transferred into the remaining portions of the sample preparation module 6200. For example, in some embodiments, the inlet coupling 6252, the outlet coupling 6253, and/or the support member 6254 can be constructed from a resilient or elastomeric material to allow vibratory movement of the bottom housing 6251 and the top housing 6260 while transferring such energy to the top body 6201.

After being mixed within the mixing assembly 6250, the prepared sample is then conveyed to the amplification module 6600. The transfer of fluids, including the reverse transcription solution, the reagents or the like is caused by the fluidic drive (or transfer) module 6400. The fluidic drive (or transfer) module 6400 can be a pump or series of pumps configured to produce a pressure differential and/or flow of the solutions within the diagnostic test device 6000. Similarly stated, the fluid transfer module 6400 is configured to generate fluid pressure, fluid flow and/or otherwise convey the biological sample and the reagents through the various modules of the device 6000. The fluid transfer module 6400 is configured to contact and/or receive the sample flow therein. Thus, in some embodiments, the device 6000 is specifically configured for a single-use to eliminate the likelihood that contamination of the fluid transfer module 6400 and/or the sample preparation module 6200 will become contaminated from previous runs, thereby negatively impacting the accuracy of the results. As shown, the fluid transfer module 6400 can be a piston pump that is coupled to the reagent module 6700 by one of the clips 6790. The fluid drive module 6400 can be driven by and/or controlled by the electronic control module 6950.

The amplification module 6600 includes a flow member 6610, a heater 6630, and a heat sink 6690. The flow member 6610 can be any suitable flow member that defines a volume or a series of volumes within which the that prepared solution S3 can flow and/or be maintained to amplify the target nucleic acid molecules within the solution S3. The heater 6630 can be any suitable heater or group of heaters coupled to the flow member 6610 that can heat the prepared solution within the flow member 6610 to perform any of the amplification operations as described herein. For example, in some embodiments, the amplification module 6600 (or any of the amplification modules described herein) can be similar to the amplification modules shown and described in U.S. Patent Publication No. 2017/0304829 entitled “Printed Circuit Board Heater for an Amplification Module,” which is incorporated herein by reference in its entirety.

In some embodiments, the flow member 6610 defines a single volume within which the prepared solution is maintained and heated to amplify the nucleic acid molecules within the prepared solution. In other embodiments, the flow member 6610 can define a “switchback” or serpentine flow path through which the prepared solution flows. Similarly stated, the flow member 6610 defines a flow path that is curved such that the flow path intersects the heater 6630 at multiple locations. In this manner, the amplification module 6600 can perform a “flow through” amplification reaction where the prepared solution flows through multiple different temperature regions.

The heater 6630 can be any suitable heater or collection of heaters that can perform the functions described herein to amplify the prepared solution. In some embodiments, the heater 6630 can establish multiple temperature zones through which the prepared solution flows and/or can define a desired number of amplification cycles to ensure the desired test sensitivity (e.g., at least 30 cycles, at least 34 cycles, at least 36 cycles, at least 38 cycles, or at least 60 cycles). The heater 6630 (and any of the heaters described herein) can be of any suitable design. For example, in some embodiments, the heater 6630 can be a resistance heater, a thermoelectric device (e.g. a Peltier device), or the like. In some embodiments, the heater 6630 can be one or more linear “strip heaters” arranged such that the flow path crosses the heaters at multiple different points. In other embodiments, the heater 6630 can be one or more curved heaters having a geometry that corresponds to that of the flow member 6610 to produce multiple different temperature zones in the flow path.

The detection module 6800 is configured to receive output from the amplification module 6600 and reagents from the reagent module 6700 to produce a colorimetric change to indicate presence or absence of target organism in the initial input sample. The detection module 6800 also produces a colorimetric signal to indicate the general correct operation of the test (positive control and negative control). In some embodiments, color change induced by the reaction is easy to read and binary, with no requirement to interpret shade or hue. The detection module 6800 can be similar to the detection modules shown and described in International Patent Publication No. WO2016/109691, entitled “Devices and Methods for Molecular Diagnostic Testing,” which is incorporated herein by reference in its entirety.

Referring to FIGS. 84 and 85, the detection module includes a lid, a detection housing 6810 and a heater 6840. The heater 6840 can be similar to any of the circuit board heaters described herein and also shown and described in International Patent Publication No. WO2016/109691, entitled “Devices and Methods for Molecular Diagnostic Testing,” which is incorporated herein by reference in its entirety. The lid and the detection housing 6810 form a flow cell for detection. The housing 6810 defines a detection chamber/channel 6812 having a sample inlet port 6814, a first reagent inlet/outlet port 6815, a second reagent inlet/outlet port 6816. The sample inlet port 6814 is fluidically coupled to the outlet of the amplification module 6600 and receives the amplified sample. The first reagent port 6815 and the second reagent port are coupled to the reagent module 6700 via the fluid interconnect 6738. Thus, in use a wash/blocking reagent (e.g., previously identified as R4) can be conveyed into the detection channel 6812 via the first reagent port 6815 or the second reagent port 6816. Similarly, a detection enzyme (e.g., previously identified as R5) and a detection substrate (e.g., previously identified as R6) can be conveyed into the detection channel 6812 via the first reagent port 6815 or the second reagent port 6816. Additionally, the first reagent port 6815 or the second reagent port 6816 can also be used to receive waste or excess reagents or flows out of the first reagent port 6815 or the second reagent port 6816.

The detection channel 6812 is surrounded or defined by a surface 6820 that includes one or more detection surfaces 6821, as well as non-detection surfaces 6826. The detection surfaces 6821 include a series of capture probes to which the target amplicon can be bound when the detection solution flows across the detection surface 6821. The capture probes can be any suitable probes formulated to capture or bind to the target amplicon. Specifically, in some embodiments, the detection portion 6821 includes five detection surfaces. Each of the detection surfaces are chemically modified to contain a desired capture probe configuration. Specifically, in some embodiments, a first detection surface can include a hybridization probe specific to Neisseria gonorrhea (NG) or Flu A. A second detection surface can include a hybridization probe specific to Chlamydia trachomatis (CT) or Flu B. A third detection surface can include a hybridization probe specific to Trichomonas vagina/is (TV), RSV, or COVID-19. A fourth detection surface can include non-target probe for a negative control. A fifth detection surface can include a hybridization probe for a positive control (A. fischeri, N. subflava, or the like).

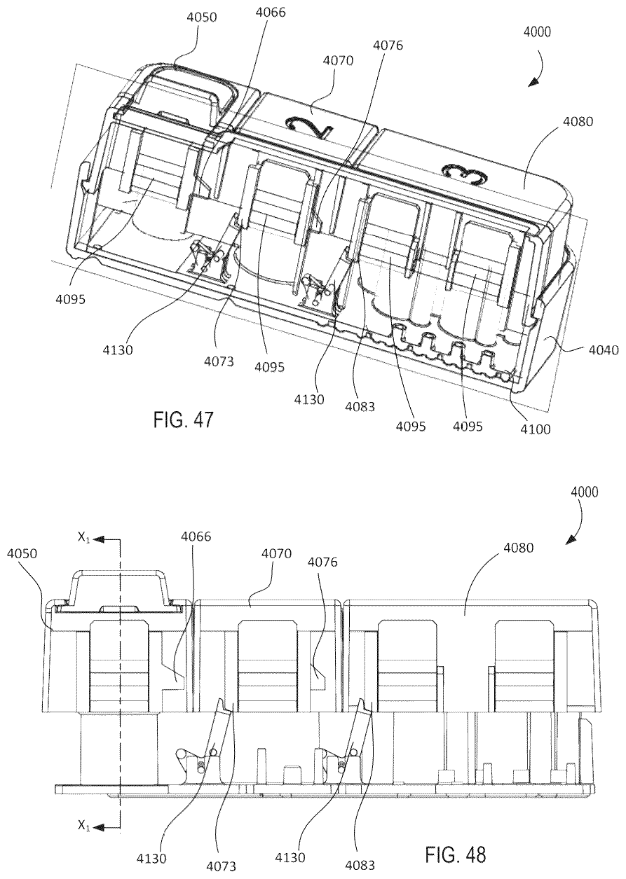

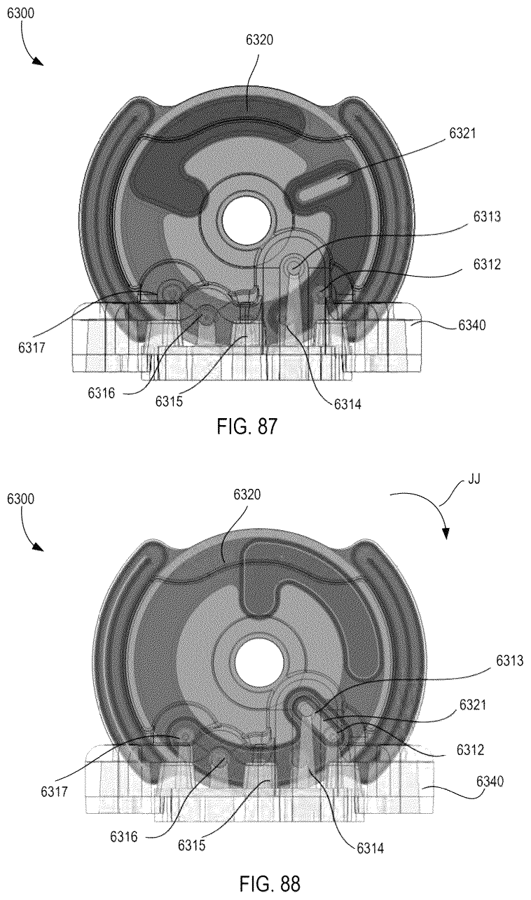

The fluid transfer valve 6300 is shown in FIGS. 59 (schematically) and 86. FIGS. 87-92 show the fluid transfer valve 6300 in several different operational configurations, with the flow (or vent) housing 6310 shown in transparent lines so that the position of the valve disk 6320 can be seen. The fluid transfer valve 6300 includes a flow housing 6310, a valve body (or disk) 6320, a main housing 6330, and a motor 6340. The flow housing 6310 defines a valve pocket within which the valve disk 6320 is rotatably disposed. The flow housing 6310 includes a flow structure that defines at least six transfer (or vent) flow paths, shown in FIGS. 47-52. Specifically, the flow paths include a sample inlet path 6312, a sample outlet path 6313, an amplification path 6314, a wash solution (reagent R4) vent path 6315, a detection enzyme (reagent R5) vent path 6316, and a detection substrate (reagent R6) vent path 6317. The flow housing 6310 includes connection portions where each of the transfer or vent paths can be coupled to the respective modules via the interconnects described herein. Each of the fluid connection/vent ports described above opens into the valve pocket. In this manner, when the valve body 6320 rotates around the center of the valve pocket (as shown by the arrow JJ), the slot channel 6321 of the valve body 6320 can connect various central ports to the other ports depending on their radial and angular position. The use of multiple radii allows not only a single port, but multiple ports at once to be fluidically coupled or vented depending on the configuration.

The valve assembly 6300 can be moved between various different configurations, depending on the angular position of the valve body 6320 within the valve pocket. FIGS. 87-92 show the assembly in various different configurations. FIG. 87 shows the valve assembly 6300 in the home (or initial position), in which the sample inlet path 6312 and the sample outlet path 6313, as well as the other fluid connection/vent ports, are closed. FIG. 88 shows the valve assembly 6300 in a first rotational position, in which the sample inlet path 6312 and the sample outlet path 6313 are opened. With the valve assembly 6300 in the first position, actuation of the fluidic drive module 6400 can produce a flow of the biological sample into and through the serpentine channel 6214 and then to the mixing assembly 6250. In this manner, the device 6000 can perform the RT-PCR methods as described herein. Moreover, the timing of the valve actuation and the power supplied to the fluidic drive module 6400 (e.g., the pump) can be controlled by the electronic control module 6950 to maintain the flow rate through the sample preparation module 6200 (including the serpentine channel 6214) within a range that the desired performance for the RT-PCR can be achieved.

After completion of the mixing process within the mixing assembly 6250, the valve assembly 6300 can be further moved into the second position (not shown). When the valve is in the second position, the amplification path 6314 is opened (i.e., is aligned with the flow slot 6321), thus allowing transfer of the mixed solution (i.e., post RT-PCR) to be conveyed into the amplification module 6600. The timing of the valve actuation and the power supplied to the fluidic drive module 6400 (e.g., the pump) can be controlled by the electronic control module 6950 to maintain the flow rate through the amplification module 6600 within a range that the desired performance for the amplification can be achieved. Moreover, with the valve assembly 6300 in the second position, continued actuation of the fluidic drive module 6400 will convey the amplified solution into and through the detection module 6800.

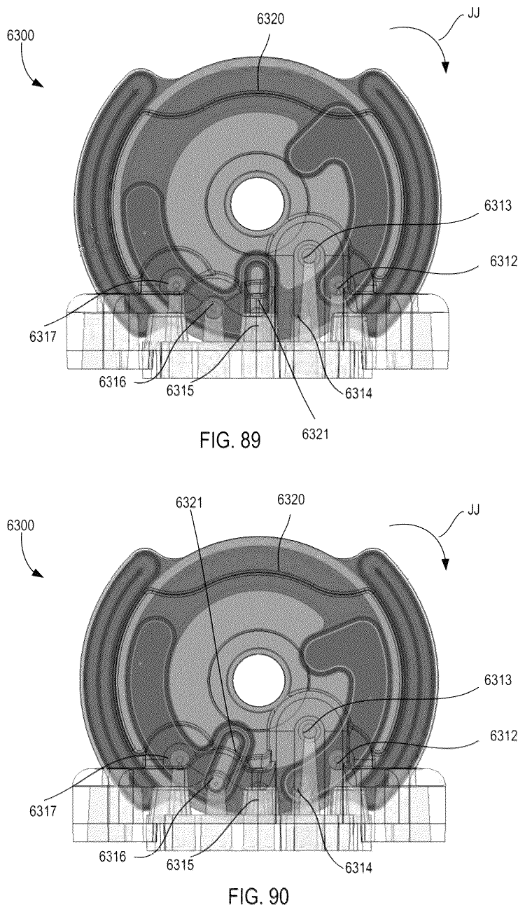

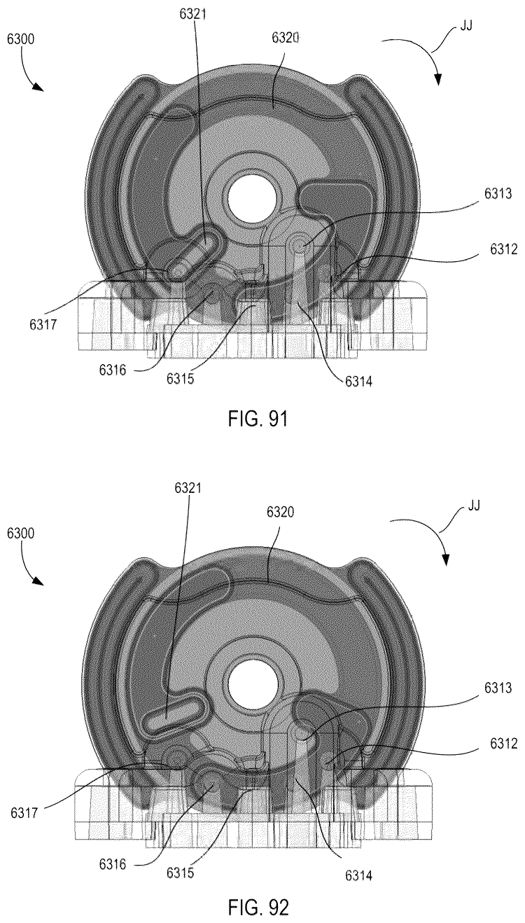

As described herein, the detection operation is accomplished by conveying a series of reagents into the detection module at specific times. Although closing the lid 6050 actuates the reagent module 6700 to open (or release) the reagents from their respective sealed containers, the reagents remain in the reagent module 6700 until needed in the detection module 6800. When a particular reagent is needed, the rotary valve 6300 opens the appropriate vent path (i.e., the wash solution vent path 6315, the detection enzyme vent path 6316, and the detection substrate vent path 6317) to the reagent module 6700. Actuation of the fluidic drive module 6400 applies vacuum to the output port of the reagent module 6700 (via the detection module 6800), thus conveying the selected reagent from the reagent module 6700 into the detection module 6800. FIG. 89 shows the valve assembly 6300 in a third rotational position, in which the detection enzyme vent path 6316 is opened. With the valve assembly 6300 in the third position, actuation of the fluidic drive module 6400 can produce a flow of the detection enzyme (reagent R5) into the detection module 6800. FIG. 90 shows the valve assembly 6300 in a fourth rotational position, in which the wash solution (reagent R4) vent path 6315 is opened. With the valve assembly 6300 in the fourth position, actuation of the fluidic drive module 6400 can produce a flow of the wash (or multi-purpose wash/blocking) solution (reagent R4) into the detection module 6800. FIG. 91 shows the valve assembly 6300 in a fifth rotational position, in which the detection substrate (reagent R6) vent path 6317 is opened. With the valve assembly 6300 in the fourth position, actuation of the fluidic drive module 6400 can produce a flow of the substrate (reagent R6) into the detection module 6800. FIG. 92 shows the valve assembly 6300 in a final position, in which the vent paths are closed.

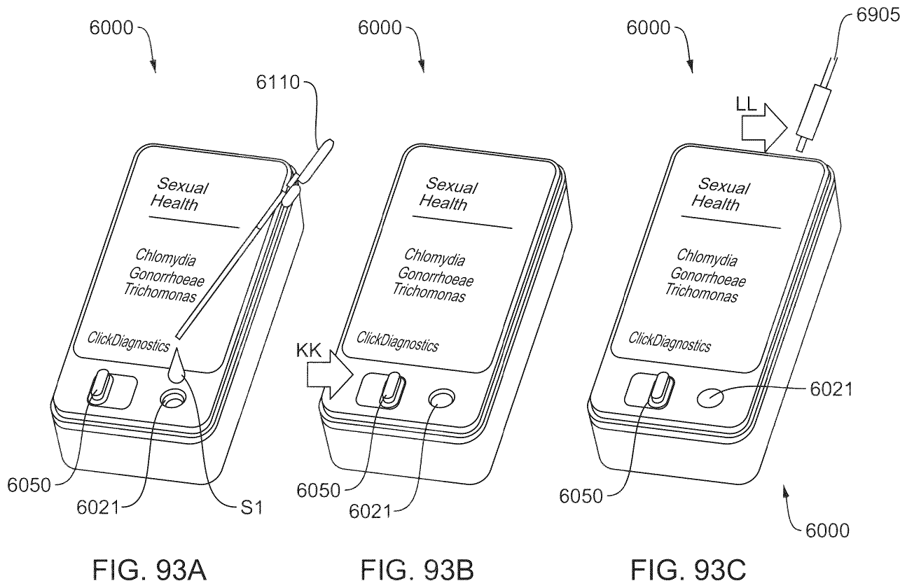

The device 6000 can be used to perform any of the methods described herein. Referring to FIGS. 93A-93C, to use the device, a biological sample S1 is first placed into the sample input opening 6021 (e.g., using a sample transfer pipette 6110), as described above. The lid 6050 is then moved to the closed position, as shown by the arrow KK in FIG. 93B. As described above, closing the lid 6050 encloses the sample input volume 6211, actuates the electronic control module 6950 (and/or the processor 6951 included therein), and also actuates the reagent module 6700, as described above. The device 6000 is then plugged in via the power cord 6905 to couple the device 6000 to a power source. In this manner, the device 6000 can, in addition to disposing the sample S1 therein and plugging in the device, be actuated by a single action (i.e., the closing of the lid).

### Kits for Detection of Target Pathogens at a Decentralized Location

The devices 4000, 6000 can be included along with any suitable accessories to facilitate conducting molecular diagnostic tests at a decentralized location. For example, in some embodiments, a kit includes a sample tube (also referred to as a dilution tube), a sample transfer device (e.g., a pastette), and any of the molecular diagnostic test devices described herein. The kit can optionally include any other suitable accessories, such as sample collection components (e.g., swabs, a sample collection tube, sterile gloves), a power adapter or power source (e.g., batteries), and disposal components (e.g., a sealable bag, wipes, etc.). The sample (or dilution) tube can include a solvent or dilution buffer, and can receive one or more biological samples. For example, in some embodiments, the dilution tube can include a suitable amount and type of buffer to receive a single biological sample (e.g., a swab sample of any type described herein taken from one patient). In other embodiments, the dilution tube can include a suitable amount and type of buffer to receive multiple biological samples (e.g., multiple swab samples of any type described herein taken from more than one patient). Thus, in some embodiments, the kit be used to perform methods of detecting a target pathogen in a combined biological sample (i.e., multiple samples that are combined) in one test event.

In some embodiments, the dilution tube contains a solvent, which comprises a buffer. The buffer may be an inorganic or an organic buffer. Non-limiting examples of buffers include phosphate buffered saline (PBS), phosphate, succinate, citrate, borate, maleate, cacodylate, N-(2-Acetamido)iminodiacetic acid (ADA), 2-(N-morpholino)-ethanesulfonic acid (MES), N-(2-acetamido)-2-aminoethanesulfonic acid (ACES), piperazine-N,N′-2-ethanesulfonic acid (PIPES), 2-(N-morpholino)-2-hydroxypropanesulfonic acid (MOPSO), N,N-bis-(hydroxyethyl)-2-aminoethanesulfonic acid (BES), 3-(N-morpholino)-propanesulfonic acid (MOPS), N-tris-(hydroxymethyl)-2-ethanesulfonic acid (TES), N-2-hydroxyethyl-piperazine-N-2-ethanesulfonic acid (HEPES), 3-(N-tris-(hydroxymethyl) methylamino)-2-hydroxypropanesulfonic acid (TAPSO), 3-(N,N-Bis[2-hydroxyethyl]amino)-2-hydroxypropanesulfonic acid (DIPSO), N-(2-Hydroxyethyl)piperazine-N′-(2-hydroxypropanesulfonic acid) (HEPPSO), 4-(2-Hydroxyethyl)-1-piperazinepropanesulfonic acid (EPPS), N-[Tris(hydroxymethyl)methyl]glycine (Tricine), N,N-Bis(2-hydroxyethyl)glycine (Bicine), (2-Hydroxy-1,1-bis(hydroxymethyl)ethyl)amino]-1-propanesulfonic acid (TAPS), N-(1,1-Dimethyl-2-hydroxyethyl)-3-amino-2-hydroxypropanesulfonic acid (AMPSO), tris (hydroxy methyl) amino-methane (Tris), TRIS-Acetate-EDTA (TAE), glycine, bis[2-hydroxyethyl]iminotris[hydroxymethyl]methane (BisTris), or combinations thereof.

In some embodiments, the buffer comprises a reducing agent. The term “reducing agent” refers to any reagent which is capable of reducing another compound in an oxidation-reduction reaction. In some embodiments, the reducing agent is selected from the group consisting of lithium, sodium, potassium, aluminum amalgam, lithium aluminum hydride, sodium borohydride, sodium cyanoborohydride, lithium tri-butoxy aluminum hydride, dibutoxy aluminum hydride, lithium triethyl borohydride, tris(2-carboxyethyl)phosphine (TCEP), dithiothreitol (DTT), mercaptoethanol, cysteine, thioglycerol, thioglycolic acid, cysteamine, glutathione, N-acetylcysteine (NAC), β-mercaptoethanol, sodium dodecyl sulfate (SDS), sodium sulfite, ammonium sulfamate, sodium bisulfite, dithionite metabisulfite sulfur dioxide, vitamin C, ascorbic acid, and dimethylsulfoxide (DMSO). In some embodiments, the reducing agent is N-acetylcysteine (NAC).

In some embodiments, the concentration of a buffer and/or reducing agent is between about 0.1 mM and 1 M, for example, between about 10 mM to about 1 M, between about 20 mM and about 500 mM, between about 50 mM and about 300 mM, between about 0.1 mM and about 50 mM, between about 1 mM and about 20 mM, between about 1 mM and about 50 mM, between about 2.5 mM and about 20 mM, including all values and subranges there between, inclusive of endpoints. In some embodiments, the concentration of the buffer and/or reducing agent is about 0.1 mM, about 0.2 mM, about 0.3 mM, about 0.4 mM, about 0.5 mM, about 0.6 mM, about 0.7 mM, about 0.8 mM, about 0.9 mM, about 1 mM, about 10 mM, about 20 mM, about 30 mM, about 40 mM, about 50 mM, about 60 mM, about 70 mM, about 80 mM, about 90 mM, about 100 mM, about 110 mM, about 120 mM, about 130 mM, about 140 mM, about 150 mM, about 160 mM, about 170 mM, about 180 mM, about 190 mM, about 200 mM, about 210 mM, about 220 mM, about 230 mM, about 240 mM, about 250 mM, about 260 mM, about 270 mM, about 280 mM, about 290 mM, about 300 mM, about 310 mM, about 320 mM, about 330 mM, about 340 mM, about 350 mM, about 360 mM, about 370 mM, about 380 mM, about 390 mM, about 400 mM, about 410 mM, about 420 mM, about 430 mM, about 440 mM, about 450 mM, about 460 mM, about 470 mM, about 480 mM, about 490 mM, about 500 mM, about 510 mM, about 520 mM, about 530 mM, about 540 mM, about 550 mM, about 560 mM, about 570 mM, about 580 mM, about 590 mM, about 600 mM, about 610 mM, about 620 mM, about 630 mM, about 640 mM, about 650 mM, about 660 mM, about 670 mM, about 680 mM, about 690 mM, about 700 mM, about 710 mM, about 720 mM, about 730 mM, about 740 mM, about 750 mM, about 760 mM, about 770 mM, about 780 mM, about 790 mM, about 800 mM, about 810 mM, about 820 mM, about 830 mM, about 840 mM, about 850 mM, about 860 mM, about 870 mM, about 880 mM, about 890 mM, about 900 mM, about 910 mM, about 920 mM, about 930 mM, about 940 mM, about 950 mM, about 960 mM, about 970 mM, about 980 mM, about 990 mM, or about 1 M, including all ranges and subranges therebetween. In some embodiments, the concentration of a buffer and/or reducing agent is about 2.5 mM. In some embodiments, the concentration of a buffer and/or reducing agent is about 5 mM. In some embodiments, the concentration of a buffer and/or reducing agent is about 7.5 mM. In some embodiments, the concentration of a buffer and/or reducing agent is about 10 mM. In some embodiments, the concentration of a buffer and/or reducing agent is about 20 mM.

As described above, in other embodiments, a reducing agent can be included within the molecular diagnostic test device (e.g., as a solid reagent stored within the sample preparation module, e.g., the sample preparation module 6200). Thus, in some embodiments, the buffer is devoid of a reducing agent. In some embodiments, any of the molecular diagnostic test devices described herein can include one or more pellets. The term “pellet” refers to a collection of dried particles that are generally spherical in shape, but may also encompasses dried particles of different shapes, including prolate spheroids, oblate spheroids, cylinders, rods, prism, or other regular geometric, or irregular shapes. The dried particles may comprise any one of or combination of a nucleic acid, an enzyme, a reducing agent, a salt, a sugar, water, solvent, buffer, or any combination thereof.

In some embodiments, the particles are dried by lyophilization. Lyophilization, also referred to as “freeze-drying,” refers to a process of removing a frozen solvent, such as ice, from a material through sublimation. In some embodiments, lyophilization is performed at a temperature between about 0.03 torr and about 0.3 torr, for example, about 0.03 torr, about 0.04 torr, about 0.05 torr, about 0.06 torr, about 0.07 torr, about 0.08 torr, about 0.09 torr, about 0.1 torr, about 0.11 torr, about 0.12 torr, about 0.13 torr, about 0.14 torr, about 0.15 torr, about 0.16 torr, about 0.17 torr, about 0.18 torr, about 0.19 torr, about 0.2 torr, about 0.21 torr, about 0.22 torr, about 0.23 torr, about 0.24 torr, about 0.25 torr, about 0.26 torr, about 0.27 torr, about 0.28 torr, about 0.29 torr, or about 0.30 torr, including all subranges there between. In some embodiments, prior to lyophilization, a composition comprising one or more of a buffer, a reducing agent, a primer, and water is prepared and aliquoted into container and frozen. In some embodiments, the container is a polyethylene terephthalate (PET) plastic tray. In some embodiments, the container is a falcon tube, a microcentrifuge tube, or a vial.

In some embodiments, pellets are produced by compressing dried particles in the form of powder with a pellet mill.

In some embodiments, the dried particles comprise enzymes. In some embodiments, the pellets comprise a mixture of enzymes, for example, 2, 3, 4, 5, 6, 7, 8, 9, 10, or more enzymes. In some embodiments, the enzyme is a horseradish peroxidase, reverse transcriptase, DNA polymerase, RNA polymerase, pyrophosphatase, and/or proteinase K. The pellet may also comprise RNase inhibitor

In some embodiments, the dried particles comprise nucleic acids. In some embodiments, the nucleic acid is RNA or DNA. In some embodiments, the nucleic acid is mitochondrial DNA, cDNA, genomic DNA, plasmid DNA, cosmid DNA, BAC, or YAC. In some embodiments, the pellet comprises deoxyribonucleotides or ribonucleotides. In some embodiment, the pellet comprises an oligonucleotide primer. In some embodiments, the primer is at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 nucleotides in length.

In some embodiments, the dried particles comprises one or more reducing agents. In some embodiments, the reducing agent is lithium, sodium, potassium, aluminum amalgam, lithium aluminum hydride, sodium borohydride, sodium cyanoborohydride, lithium tri-butoxy aluminum hydride, dibutoxy aluminum hydride, lithium triethyl borohydride, tris(2-carboxyethyl)phosphine (TCEP), dithiothreitol (DTT), mercaptoethanol, cysteine, thioglycerol, thioglycolic acid, cysteamine, glutathione, N-acetylcysteine (NAC), β-mercaptoethanol, sodium dodecyl sulfate (SDS), sodium sulfite, ammonium sulfamate, sodium bisulfite, dithionite metabisulfite sulfur dioxide, vitamin C, ascorbic acid, dimethylsulfoxide (DMSO), or any combination thereof. In some embodiments, the dried particles comprise NAC. In some embodiments, the N-acetylcysteine (NAC) is N-acetyl-L-cysteine. In some embodiments, the N-acetylcysteine is N-acetyl-D-cysteine.

In some embodiments, the dried particles comprise a lyophilization buffer.

In some embodiments, the dried particles comprise NAC. In some embodiments, the dried particles comprise NAC and an oligonucleotide (e.g., a primer). In some embodiments, the dried particles comprise NAC, an oligonucleotide, and a buffer. In some embodiments, the dried particles comprise NAC, an oligonucleotide, a buffer, and an enzyme. In some embodiments, the dried particles comprise an enzyme and nucleotides.

In some embodiments, any of the devices and kits described herein can include the CDC N1 and N2 primer and probe sets. Based on analysis of characteristics including Tm, amplicon size, and second structure of the target), it is believed that these primer and probe sets will likely function the best on the fast-cycling conditions produced by the devices described herein. The primer pellet makes use of the N1 and N2 primer sets from the CDC assay and targets the nucleocapsid gene of the SARS-CoV-2 genome that effectively discriminates it from SARS-CoV. The CDC primer set for RNAse P is also included in the primer pellet, which will serve as a sample adequacy control and an end-to-end process control for the test.

TABLE 1
Primer and probe sequences

| Target | Primers | Probe | Amplicon Size |
|---|---|---|---|
| SARS-CoV- | 2019-nCoV_N1-F | 2019-nCoV_N1-P-C | 72 bp |
| 2 (N1 assay) | 5′-GAC CCC AAA ATC AGC GAA | 5′-amino linker-ACC CCG | |
| | AT-3′ | CAT TAC GTT TGG ACC- | |
| | 2019-nCoV_N1-R | 3′ | |
| | 5′-biotin-TCT GGT TAC TGC CAG | | |
| | TTG AAT CTG-3′ | | |
| SARS-CoV- | 2019-nCoV_N2-F | 2019-nCoV_N2-P | 67 bp |
| 2 (N2 assay) | 5′-TTA CAA ACA TTG GCC GCA | 5′- amino linker -ACA ATT | |
| | AA-3′ | TGC CCC CAG CGC TTC | |
| | 2019-nCoV_N2-R | AG -3′ | |
| | 5′-biotin-GCG CGA CAT TCC GAA | | |
| | GAA-3′ | | |
| RNAse P | RP-F | RP-P-C | 65 bp |
| | 5′-AGA TTT GGA CCT GCG AGC | 5′-amino linker-TTC TGA | |
| | G-3′ | CCT GAA GGC TCT GCG | |
| | RP-R | CG-3′ | |
| | 5′-biotin-GAG CGG CTG TCT CCA | | |
| | CAA GT-3′ | | |

### Methods and Devices Using a RT-PCR Device to Detect RNA Viruses

In some embodiments, any of the devices described herein can be used to perform a single-use (disposable), point-of-need, diagnostic test for detecting one or more of Influenza A (Flu A), Influenza B (Flu B), Coronavirus (e.g., novel coronaviruses like SARS-CoV-2), and Respiratory Syncytial Virus (RSV) from a nasal swab sample. This will assist clinicians in identifying patients better served by antivirals, thus reducing the prescription of unnecessary and ineffective antibiotics that lead to antimicrobial resistance.

In some embodiments, the test device (and methods) can include a nasal swab and can be conducted on any of the devices described herein. In some embodiments, a method can include performing a nasal wash to produce a sample for input into any of the devices described herein.

In some embodiments, a single molecular diagnostic test device can be used to screen multiple patients at one time. For example, in some embodiments, a method of screening can include combining biological samples from a set of patients to form a combined sample. The combined sample is then analyzed using any of the devices or methods described herein. The signal produced can be read to determine whether the target virus (e.g., coronavirus) is present in the combined sample. When the test is positive, each of the patients is then tested individual (or via a narrow set of parallel screening). If, however, the test is negative, the entire test group can be identified as negative without requiring individual tests. Because of the advantageous limit of detection described herein, the method can accurately detect a positive sample from one patient that is diluted with several other negative samples from the other patients. In some embodiments, the number of patients that can be tested in a single device is five or less. In. In other embodiments, the number of patients that can be tested in a single device as many as 10. In yet other embodiments, the number of patients that can be tested in a single device as many as 20. Moreover, in some embodiments, the intensity (or magnitude) of the signal can be evaluated to determine a range of patients within the screened group that is positive.

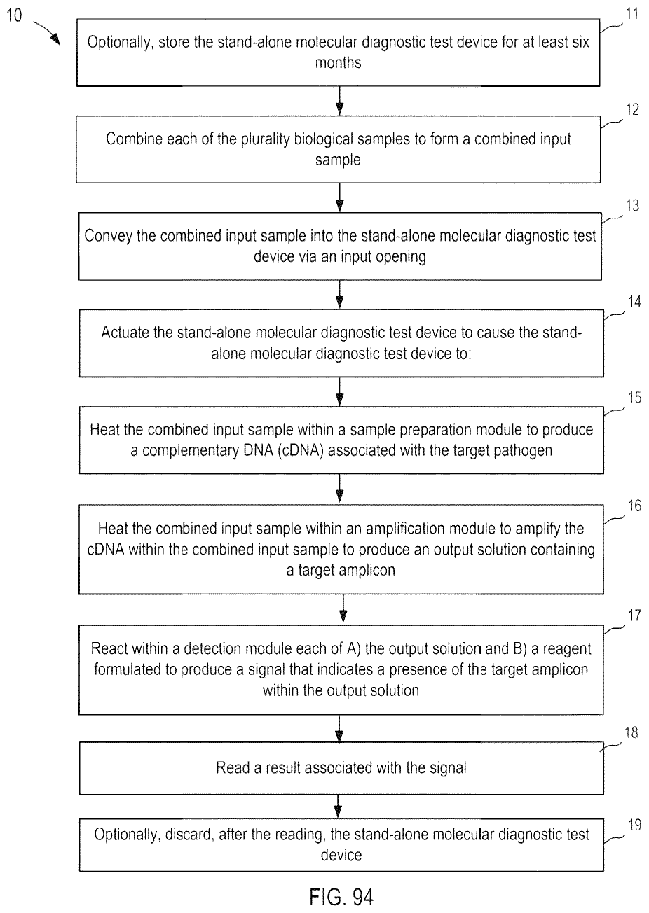

FIG. 94 is a flow chart of a method 10 of detecting a target pathogen in a combined (or pooled) biological sample using a stand-alone molecular diagnostic test device, according to an embodiment. The method 10 can be performed on any suitable device, such as the device 4000 or the device 6000. The method 10 can optionally include storing the stand-alone molecular diagnostic test device for at least six months prior to use, at 11. The method 10 includes combining each of the biological samples to form a combined input sample, at 12. In some embodiments, one or more of the biological samples is a swab sample. For example, in some embodiments, one or more of the biological samples is collected from one of a nasal swab, a mid-turbinate swab, a nasopharyngeal swab, or an oropharyngeal swab. In some embodiments, the combining includes conveying each of the swab samples into a single dilution tube containing transport media and/or a buffer of any of the types described herein. The combined sample is then conveyed into the stand-alone molecular diagnostic test device. This approach can be referred to as “swab pooling,” and can conserve the transport media and/or buffers, but may also result in a higher concentration swab materials, proteins, mucin, and the target pathogen (if there are multiple positive samples among the biological samples). As described herein, the devices and methods described herein can accommodate the higher concentration of potentially inhibitory constituents, and can still produce a high sensitivity and high specificity.

In some embodiments, the combining includes conveying each of the biological samples into a separate collection tube containing transport media and/or a buffer of any of the types described herein. The contents of each of the collection tubes are then mixed together to produce a combined sample. This approach can be referred to as “sample pooling,” and can allow multiple different samples to be tested in a single operation, but may also result in reduced sensitivity due to the dilution of each of the samples. For example, if one of the biological samples is a “weak” positive sample, but including it in with additional volumes of media and other sample, it is possible that the one weak positive sample may not be detected. As described herein, however, the devices and methods described herein can accommodate handling pooled samples, and can still produce a high sensitivity and high specificity.

In some embodiments, the number of biological samples includes between two biological samples and twenty biological samples. In some embodiments, the number of biological samples includes between three biological samples and ten biological samples. In some embodiments, the number of biological samples includes between four biological samples and eight biological samples. In some embodiments, the number of biological samples is five biological samples.

The combined input sample is conveyed into the stand-alone molecular diagnostic test device via an input opening, at 13. In some embodiments, the combined input sample can be conveyed into the device by a sample transfer device, such as a pastette.

The molecular diagnostic test device is then actuated, at 14, which causes the molecular diagnostic test device to perform a series of operations without any further user input. In some embodiments, the molecular diagnostic test device is actuated via only a “single button,” as shown above for the device 6000. After being actuated, the molecular diagnostic test device can perform any of the operations described herein. Specifically, the device can heat the combined input sample within a sample preparation module to produce a complementary DNA (cDNA) associated with the target pathogen, at 15. For example, the device can heat the combined input sample within a sample preparation module or reverse transcription module as described herein.

The combined input sample is then heated within an amplification module (e.g., the amplification module 6600) to amplify the cDNA within the combined input sample to produce an output solution containing a target amplicon, at 16. The combined input sample can be amplified by using any suitable technique (e.g., PCR, isothermal amplification, etc.), as described herein.

After amplification, the device then reacts within a detection module within the molecular diagnostic test device each of (i) the output solution and (ii) a reagent formulated to produce a signal that indicates a presence of the target amplicon within the output solution, at 17. As described herein, the detection module can be the detection module 6800 that includes one or more detection surfaces configured to capture the target amplicon to produce the output signal. The output signal can be any suitable signal. In some embodiments, the output signal can be a colorimetric signal that indicates the presence of bound amplicon: if the target pathogen, target amplicon and/or target organism is present, the color product is formed, and if the target pathogen, target amplicon and/or target organism is not present, the color product does not form. The method then includes reading a result associated with the signal, at 18.

In some embodiments, the method optionally includes discarding, after the reading, the stand-alone molecular diagnostic test device, at 19. The discarding can include sealing the device within a bag or container and disposing of the device in standard waste streams. In other embodiments, the device can be returned to a processing facility. For example, in some embodiments, the method can include sealing the device within a bag or container and sending the device (e.g., via mail, courier or other shipping channel) to a central facility. In this manner, the device, including any chemicals, reagents, or electronic component can be disposed of, treated, and/or recycled in a suitable manner. Moreover, this procedure can allow for other data verification (e.g., confirmation of a test result stored on a processor within the device).

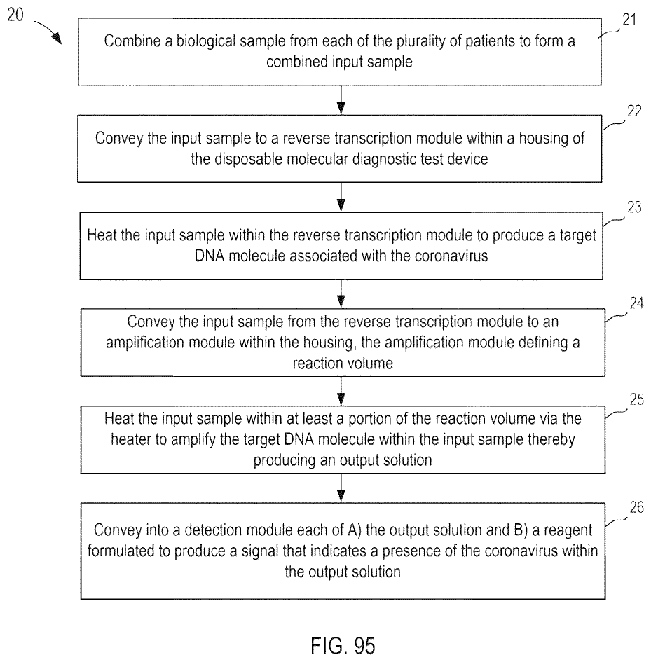

FIG. 95 is a flow chart of a method 20 of detecting a coronavirus in a combined (or pooled) biological sample using a stand-alone molecular diagnostic test device, according to an embodiment. The method 20 can be performed on any suitable device, such as the device 4000 or the device 6000. The method 20 can optionally include storing the stand-alone molecular diagnostic test device for at least six months prior to use. The method 20 includes combining each of the biological samples to form a combined input sample, at 21. In some embodiments, one or more of the biological samples is a swab sample. For example, in some embodiments, one or more of the biological samples is collected from one of a nasal swab, a mid-turbinate swab, a nasopharyngeal swab, or an oropharyngeal swab. In some embodiments, the combining includes conveying each of the swab samples into a single dilution tube containing transport media and/or a buffer of any of the types described herein. The combined sample is then conveyed into the stand-alone molecular diagnostic test device. In other embodiments, the combining includes conveying each of the biological samples into a separate collection tube containing transport media and/or a buffer of any of the types described herein. The contents of each of the collection tubes are then mixed together to produce a combined sample.

The input sample is conveyed to a reverse transcription module within a housing of the stand-alone molecular diagnostic test device, at 22. The input sample is heated within the reverse transcription module to produce a target DNA molecule associated with the coronavirus, at 23. The input sample is then conveyed from the reverse transcription module to an amplification module within the housing, at 24. The amplification module can be any of the amplification modules described herein, and defines a reaction volume.

The input sample is heated within at least a portion of the reaction volume via the heater to amplify the target DNA molecule within the input sample thereby producing an output solution, at 25. The method includes conveying into a detection module each of A) the output solution and B) a reagent formulated to produce a signal that indicates a presence of the coronavirus within the output solution, at 26.

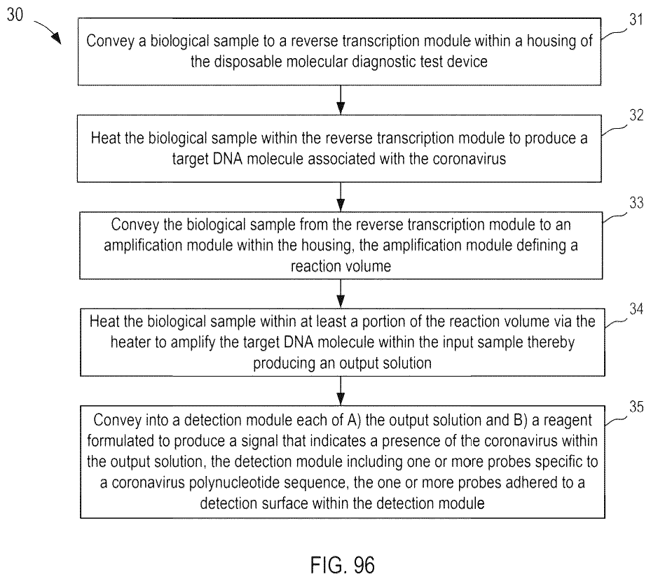

FIG. 96 is a flow chart of a method 30 of detecting a coronavirus using a disposable molecular diagnostic test device, according to an embodiment. The method 30 can be performed on any suitable device, such as the device 4000 or the device 6000. The method 30 can optionally include storing the disposable molecular diagnostic test device for at least six months prior to use. The method 30 includes conveying a biological sample to a reverse transcription module within a housing of the disposable molecular diagnostic test device, at 31. The biological sample is heated within the reverse transcription module to produce a target DNA molecule associated with the coronavirus, at 32. The biological sample is then conveyed from the reverse transcription module to an amplification module within the housing, at 33. The amplification module can be any of the amplification modules described herein, and defines a reaction volume.

The biological sample is heated within at least a portion of the reaction volume via a heater to amplify the target DNA molecule within the input sample thereby producing an output solution, at 34. The method includes conveying into a detection module each of A) the output solution and B) a reagent formulated to produce a signal that indicates a presence of the coronavirus within the output solution, at 35.

### Example 1—Demonstration of Flu A/B

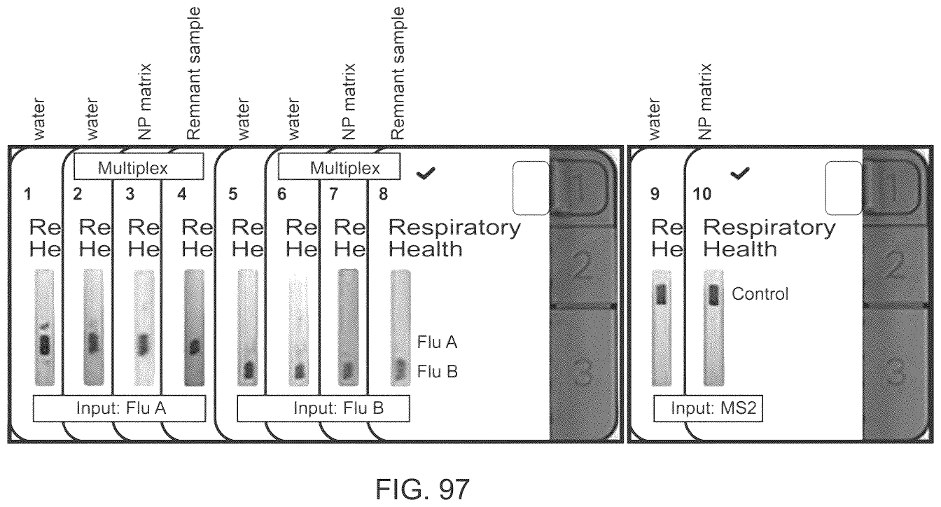

The Flu A/B RT-PCR primers were derived from those published by the CDC, but were modified to function on the stand-alone devices as described herein. Candidate primers and capture probes were first bioinformatically evaluated using an internal genome sequence database of Flu A (˜30,000 strains) and Flu B (˜17,000 strains) to ensure adequate coverage of strains to meet rigorous “inclusivity” requirements. The primer sets were tested on the benchtop under slow and fast cycling conditions and the best performing set (58 bp Flu A amplicon corresponding to a conserved region of the M gene; 75 bp Flu B amplicon corresponding to a conserved region of the HA gene) were selected to be empirically tested on a device similar to the device 4000 described herein. FIG. 97 is a series of photographs showing the detection opening portion of each of the devices, which are labeled as device 1-8.

Several elements of the on-device process were empirically determined: 1) optimal influenza virus lysis time and temperature; 2) optimal RT reaction time; 3) functionality of the assay in viral transport medium; 4) functionality of the primers in multiplex; and 5) functionality of capture probes on the flow cell. Alignment of the Flu A primer set and capture probe against our Flu A database showed that they correspond to a conserved region of the vast majority of Flu A M gene sequences and thus would predictably detect past and present circulating strains, including, seasonal and pandemic subtypes (Devices 1-4, FIG. 97). Similarly, alignment of the Flu B primer set and capture probe to a Flu B database showed that these correspond to a conserved region of the HA gene of circulating Flu B strains (Devices 5-8 FIG. 97). Viral transport medium was found to be slightly inhibitory of RT-PCR, an effect that could be mitigated by a 1:1 dilution in a standard transport medium (FIG. 97). The entire on-device time for the non-optimized process from sample processing to detection is ˜ 30 min.

### Example 2—Demonstration of Analytical Performance

This example demonstrates operation of COVID-19 according to device 4000 as an in vitro diagnostic for the qualitative detection of viral RNA from the SARS-CoV-2. Use this test with nasopharyngeal, nasal, or mid-turbinate swabs collected by a health care provider (HCP), or nasal or mid-turbinate swabs self-collected (in a healthcare setting) eluted in viral transport medium and diluted in a suitable solution.

Limit of Detection (LoD) studies were performed to determine the analytical LoD of device 4000. Dilutions of inactivated SARS-CoV-2 (USA_WA1/2020 strain) in negative nasopharyngeal clinical matrix were tested in replicates of 20. The LoD value was estimated by a probit analysis of the results from the range-finding study. Verification of the estimated LoD was performed by testing 20 replicates at the estimated concentration and confirming the est detected the inactivated SARS-CoV-2 virus ≥95% of the time. The LoD of the test for SARS-CoV-2 virus is 1,112 genomic copies/mL (Table 1).

TABLE 1
LoD Determination using inactivated
SARS-CoV-2 (USA_WA1/2020 strain)

| Inactivated SARS- | Concentration | | |
|---|---|---|---|
| CoV-2 Virus | (genomic | | |
| (USA_WA1/2020) | copies/mL) | Detected/Tested | % Detected |
| Range Finding | 125 | 9/20 | 45% |
| | 250 | 11/20 | 55% |
| | 500 | 15/20 | 75% |
| | 750 | 18/20 | 90% |
| | 1000 | 20/20 | 100% |
| Verification | 1112 | 19/20 | 95% |

In addition, testing was also performed to evaluate the device test performance when in the presence of 31 viral and bacterial organisms. Each organism was individually seeded into an artificial nasal matrix and tested on three devices with both COVID-19 negative samples and COVID-19 positive samples at 2× the LOD. The expected results were achieved 100% of the time, allowing for a re-test of one sample. The organisms, concentrations and results are listed below. None of the 31 organisms caused cross-reactivity on the test at the concentrations in Table 2.

TABLE 2
Summary of performance (Cross-Reactivity and Microbial Interference)

| | | | Negative | Positive |
|---|---|---|---|---|
| | | | Samples | Samples |
| | | | (# of Valid | (# of Valid |
| | | | Devices | Devices |
| | Concentration | | Negativefor | Positive for |
| Organism | Tested | Units | SARS-CoV-2) | SARS-CoV-2) |
| Human Coronavirus NL63 | 1.1 × 10⁵ | genomic | 3/3 | 3/3 |
| | | copies/mL | | |
| SARS-Coronavirus (2003) | 1.1 × 10⁵ | genomic | 3/3 | 3/3 |
| | | copies/mL | | |
| MERS-Coronavirus | 1.1 × 10⁵ | genomic | 3/3 | 3/3 |
| | | copies/mL | | |
| Adenovirus, C1 Ad 71 | 2.5 × 10⁻³ | ng/μL | 3/3 | 3/3 |
| Human metapneumovirus | 1.1 × 10⁵ | genomic | 3/3 | 3/3 |
| (hMPV) | | copies/mL | | |
| Human parainfluenza virus 1 | 2.5 × 10⁻³ | ng/μL | 3/3 | 3/3 |
| Human parainfluenza virus 2 | 2.5 × 10⁻³ | ng/μL | 3/3 | 3/3 |
| Human parainfluenza virus 3 | 2.5 × 10⁻³ | ng/μL | 3/3 | 8/9 (2) |
| Human parainfluenza virus 4b | 2.5 × 10⁻³ | ng/μL | 3/3 | 3/3 |
| Influenza A | 1.1 × 10⁶ | CEID50/mL | 3/3 | 3/3 |
| Influenza B | 1.1 × 10⁶ | CEID50/mL | 3/3 | 3/3 |
| Enterovirus 68 | 1.1 × 10⁵ | genomic | 3/3 | 3/3 |
| | | copies/mL | | |
| Respiratory syncytial virus | 1.1 × 10⁵ | genomic | 3/3 | 3/3 |
| | | copies/mL | | |
| Human rhinovirus 17 (strain | 1.1 × 10⁵ | genomic | 3/3 | 3/3 |
| 33342) | | copies/mL | | |
| Chlamydia pneumoniae | 1.1 × 10⁶ | IFU/mL | 3/3 | 3/3 |
| Haemophilus influenzae | 1.1 × 10⁶ | genomic | 3/3 | 3/3 |
| | | copies/mL | | |
| Legionella pneumophila | 1.1 × 10⁶ | genomic | 3/3 | 3/3 |
| | | copies/mL | | |
| Mycobacterium tuberculosis | 1.1 × 10⁶ | genomic | 3/3 | 3/3 |
| | | copies/mL | | |
| Streptococcus pneumoniae | 1.1 × 10⁶ | genomic | 3/3 | 3/3 |
| | | copies/mL | | |
| Streptococcus pyogenes | 1.1 × 10⁶ | genomic | 3/3 | 3/3 |
| | | copies/mL | | |
| Bordetella parapertussis | 1.1 × 10⁶ | genomic | 3/3 | 3/3 |
| | | copies/mL | | |
| Mycoplasma pneumoniae | 1.1 × 10⁶ | genomic | 3/3 | 3/3 |
| | | copies/mL | | |
| Pneumocystis jirovecii (PJP), | 1.1 × 10⁶ | nuclei/mL | 3/3 | 3/3 |
| also called: | | | | |
| Pneumocystis carinii Delanoe | | | | |
| and Delanoe | | | | |
| Candida albicans | 1.1 × 10⁶ | genomic | 3/3 | 3/3 |
| | | copies/mL | | |
| Pseudomonas aeruginosa | 1.1 × 10⁶ | genomic | 3/3 | 3/3 |
| | | copies/mL | | |
| Staphylococcus epidermis | 1.1 × 10⁶ | genomic | 3/3 | 3/3 |
| | | copies/mL | | |
| Streptococcus salivarius | 1.1 × 10⁶ | genomic | 3/3 | 3/3 |
| | | copies/mL | | |
| Pooled human nasal wash | 10% | percent of total | 3/3 | 3/3 |
| | | volume | | |

A study was executed to determine the effect of endogenous and exogenous potentially interfering substances that may be present in a clinical sample on the performance of the test. Each potential interfering substance was seeded into negative nasopharyngeal clinical matrix and tested in triplicate. Each potential interfering substance was also seeded into the negative nasopharyngeal clinical matrix spiked with inactivated SARS-CoV-2 virus at 2×LoD and tested in triplicate. The substances, concentrations and results are listed below. It was determined that the worst case was represented by a mid-turbinate swab saturated with the interferent. The swab is capable of holding a maximum of 75 μL resulting in a final maximum concentration post-elution of 2.5% (v/v). None of the substances tested for interference impacted the performance or results of the test at the concentrations in Table 3.

TABLE 3
Summary of valid device performance
for each interfering substance

| | | | Negative Samples | Positive Samples |
|---|---|---|---|---|
| | | | # Negative for | # Positive for |
| Interfering | | | SARS-CoV-2/ | SARS-CoV-2/ |
| Substance | Concentration | | # Tested | # Tested |
| Mucin | 1% | (w/v) | 3/3 | 3/3 |
| Zanamivir | 282 | ng/ml | 3/3 | 3/3 |
| (Relenza) | | | | |
| Biotin | 3.5 | μg/mL | 3/3 | 3/3 |
| Mupirocin | 12 | mg/mL | 3/3 | 3/3 |
| Tobramycin | 2.43 | mg/mL | 3/3 | 3/3 |
| Afrin | 2.5% | (v/v) | 3/3 | 3/3 |
| Fresh Whole | 5% | (v/v) | 3/3 | 3/3 |
| Blood Pooled | | | | |
| Human Donors | | | | |
| Flumist (3) | 2.5% | (v/v) | 3/3 | 3/3 |
| Flonase | 2.5% | (v/v) | 3/3 | 3/3 |
| Nasacort | 2.5% | (v/v) | 3/3 | 3/3 |
| Nasal Saline | 2.5% | (v/v) | 3/3 | 3/3 |
| Spray | | | | |
| NeoSynephrine | 2.5% | (v/v) | 3/3 | 3/3 |
| Cold & Sinus | | | | |
| ExtraStrength | | | | |
| Spray | | | | |
| Zicam | 2.5% | (v/v) | 3/3 | 3/3 |
| Allergy | | | | |
| Relief | | | | |

Clinical Evaluation: The objective of this study was to establish the performance characteristics of the test as compared to an EUA-authorized test in clinical specimens. A total of sixty-three (63) samples were tested in the study. Of these, three (3) yielded invalid results during the initial test and sufficient volume in the original sample was not available for a retest. Thus, sixty (60) samples were included in the final dataset for the analysis. Specimens were randomized and blinded to the study operators. Performance estimates for Positive Percent Agreement (PPA) and Negative Percent Agreement (NPA) are shown in Table 4.

Relative to the EUA-authorized comparator test, the Visby COVID-19 test demonstrated both PPA and NPA for detection of SARS-CoV-2 RNA of 100% (95% CI: 88.6%-100.0%).

TABLE 4
Visby COVID-19 test vs EUA-authorized Comparator Assay

EUA-authorized Test

| | | POS | NEG | TOTAL |
|---|---|---|---|---|
| Test | POS | 30 | 0 | 30 |
| Device | NEG | 0 | 30 | 30 |
| | TOTAL | 30 | 30 | 60 |

| | |
|---|---|
| PPA | 100% (95% CI: 88.6%-100.0%) |
| NPA | 100% (95% CI: 88.6%-100.0%) |

The evaluation of sensitivity and MERS-CoV cross-reactivity was performed using reference material (TI), blinded samples and a standard protocol provided by the FDA. The study included a range finding study and a confirmatory study for LoD. Blinded sample testing was used to establish specificity and to confirm the LoD. The samples were tested with the test device 4000. The results are summarized in Table 5. (NDU/mL=RNA NAAT detectable units/mL; N/A: Not applicable; ND: Not detected)

TABLE 5
Summary of LoD Confirmation Result using
the FDA SARS-CoV-2 Reference Panel

| Reference Materials | Specimen | Product | Cross- |
|---|---|---|---|
| Provided by FDA | Type | LoD | Reactivity |
| SARS-CoV-2 | Nasopharyngeal | 5.4 × 10⁴ NDU/mL | N/A |
| MERS-CoV | Swab | N/A | ND |

### Example 3—Demonstration of Combined Samples

These examples demonstrate that inclusion of a reducing agent increases the performance of a stand-alone molecular diagnostic test device by reducing additive inhibitory effects of patient samples. The increased sensitivity of the device enables reliable detection of nucleic acids associated with a pathogen (e.g., coronavirus) in pooled samples—such as, in particular, pooled nasopharyngeal swabs samples from 5, 10, or more swabs.

Dose-Dependent Improvement in Amplification with NAC

The purpose of this study is to assess how NAC affects inhibition from different concentrations of pooled patient samples. A concentrated negative sample matrix (NSM) pool was generated by pumping several freshly collected Dual Anterior Nasal swabs from presumed negative patients directly into a single sample tube containing RT-PCR buffer (FB5). The concentrated NSM pool was then diluted with clean RT-PCR buffer to the equivalent of 10 swabs per sample, 5 swabs per sample, and 1 swab per sample. Also included is a no NSM control where there were no patient swabs in the sample to replicate a sample with no added inhibition. NAC solution was added to each sample at either 0 mM, 2.5 mM, 5 mM, 10 mM or 20 mM final concentration. All samples were spiked with the same concentration of 200 copies/uL of inactivated SARS-CoV-2 virus then run on benchtop RT-qPCR targeting the N1 gene of the virus. RT master mix was spiked into each sample at 5% sample volume and the samples were incubated at 60° C. for two minutes then 95° C. for 2 minutes. The RT output was mixed 1:1 with PCR master mix and run on the LightCycler480. Final concentration of virus in PCR for all samples was 100 copies/uL.

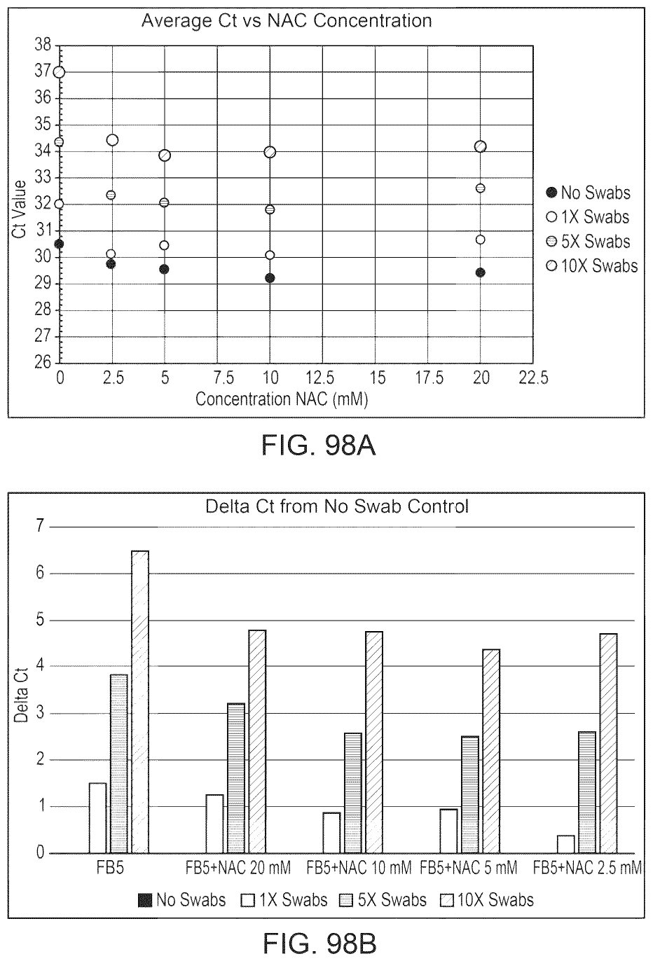

FIG. 98A show output Cycle Threshold (Ct) values, assessed by amplification of input solution by quantitative PCR on a LightCycler. Since all reactions had the same target input concentration differences in Ct can be attributed to increased performance of the assay. Average Ct values increase with increasing NSM concentration (1× to 10× swab equivalents), demonstrating that at higher swab concentrations the device generates lower levels of amplified DNA for detection in the detection module. Addition of NAC restores lower Ct values. Specifically, 5× swab equivalents is amplified to the same extent as 1× swab equivalents without NAC; 10× swab equivalents is amplified to the same extent as 5× swab equivalents without NAC. The same data is plotted in bar graph form in FIG. 98B. In conclusion, NAC improves RT-PCR by reducing concentration-dependent inhibition.

Improvement in Amplification with NAC on a Stand-Alone Device

A concentrated NSM pool was generated by pumping several freshly collected Dual Anterior Nasal swabs from presumed negative patients directly into a single sample tube containing RT-PCR buffer (FB5). The sample was split in half and diluted to the equivalent of 5 swabs per sample for the final NSM concentration. NAC solution was added to one half of the sample for the final concentration of 5 mM NAC, while the other half of the sample did not receive NAC and was representative of our point of reference (POR) control. Both samples were spiked with 375 copies/mL (5×LOD determined with 1×NSM) of inactivated SARS-CoV-2 virus. The samples were loaded on n=10 COVID-19 devices similar to the device 4000 described above per condition and the devices were run to completion as per our normal device run protocol. After the devices were run they were assessed visually for spot intensity and drop outs and additionally were subject to rePCR where the PCR output of each device is diluted 1:1000 and inputted into a qPCR protocol to yield a Ct value.

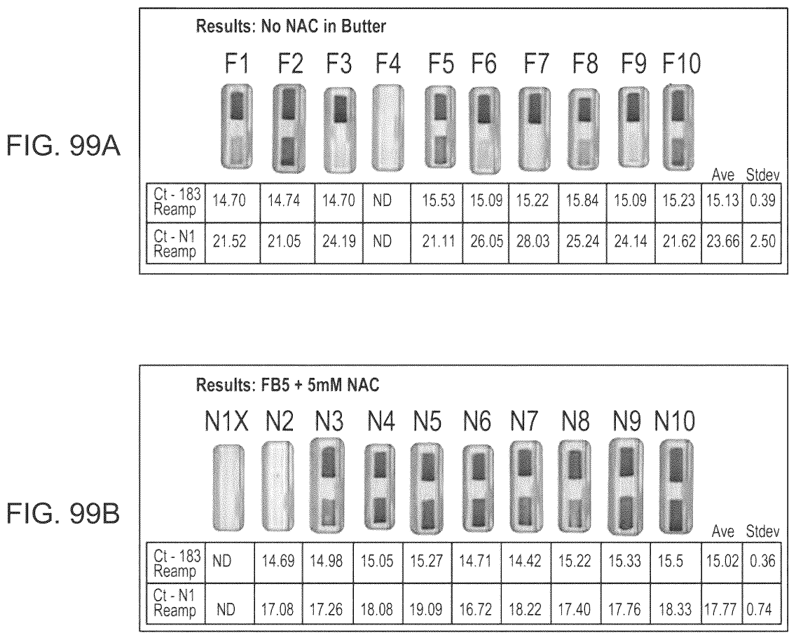

FIG. 99A shows detection of coronavirus genomic RNA in a stand-alone molecular diagnostic test device (similar to the device 4000) without the reducing agent present in the buffer. Testing of samples at 5× swab equivalents at 5×LOD target input, without reducing agent, generates variable signal intensity and unfavorably high rePCR Ct values. Note: device F4 had issues unrelated to lack of NAC.

FIG. 99B shows detection of coronavirus genomic RNA in a stand-alone molecular diagnostic test device (similar to the device 4000) with NAC present in the buffer. Testing of samples at 5× swab equivalents, with reducing agent (NAC), generates far less variable signal intensity and lower rePCR Ct values. The average Ct decrease from POR as seen in FIG. 99B is 5.89 indicating that NAC greatly improved device performance at 5×LOD with 5×NSM. Note: devices N1 and N2 had performance issues unrelated to the presence of NAC in the buffer.

Improvement in Amplification with NAC on a Stand-Alone Device

### Preparation of Pelletized Reducing Agent

To permit inclusion of the reducing agent within the manufactured handheld device (e.g., similar to the device 4000 or the device 6000), rather than external to the device, we developed a method to include the reducing agent in a pellet. In this example, the reducing agent N-acetyl-L-cysteine (NAC) was included in a pellet with oligonucleotide primers (the reverse primers) and other buffer components. The enzyme necessary for RT-PCR (reverse transcriptase) is supplied in a second pellet along with dNTP's.

To generate NAC-containing pellets, a liquid bulk containing NAC and oligonucleotide primers was made at 26.5× concentration so when the pellets were rehydrated with the sample during test processing the components were at 1× (Table 6).

TABLE 6

| | Amount | | Concentration | | |
|---|---|---|---|---|---|
| | Per | | After | | |
| Component | Pellet | | Rehydration | | Purpose of Component |
| N-acetylcysteine | 0.53 | mg | 5 | mM | Reducing agent |
| Lyophilization | 16.95 | uL | 0.3X | | Contains sugars that provide structure |
| Buffer | | | | | for the pellets once lyophilized |
| N1-Rev | 1.7 | uL | 800 | nM | Targets N gene in SARS-CoV-2 genome |
| Primer | | | | | for detection of pathogen in RT and PCR |
| 18S-Rev | 0.05 | uL | 25 | nM | Targets 18S RNA in human cells serving |
| Primer | | | | | as process control in RT and PCR |

| | | | | |
|---|---|---|---|---|
| Water | 1.01 | uL | N/A | Used to dilute components to correct |

concentrations

The components were fully dissolved and mixed then the bulk was dispensed into 20 μL aliquots on a polyethylene terephthalate (PET) plastic tray that serves as a mold to keep the pellets shape as they are lyophilized. The filled plastic trays were placed on a pre-frozen aluminum tray that instantly freezes the liquid in the plastic trays. This immediate freeze served to avoid unwanted ice crystal formation that would occur if the liquid was slowly frozen. The frozen trays were then loaded into the lyophilizer where combinations of temperature changes and vacuum pressure pull all of the liquid out of the pellets leaving behind only the solid components that were once dissolved.

### TESTING OF HANDHELD (STAND-ALONE) DEVICE CONTAINING NAC PELLETS

Lyophilized NAC pellets (without primers) were generated at a 0 mM NAC, 2.5 mM NAC, and 5 mM NAC according to the method as described above. These pellets were added to the device sample port of COVID-19 test devices (similar to the device 6000 described herein) prior to inputting sample.

A concentrated NSM pool was generated by pumping several freshly collected Dual Mid-turbinate swabs from presumed negative patients directly into a single sample tube containing RT-PCR buffer (FB5) to generate the equivalent of 3 swabs per sample. The sample pool was spiked with 225 copies/mL (3×LOD determined with 1×NSM) then loaded on to n=7 devices per condition. The devices were run to completion via our normal device run protocol. After the devices were run they were assessed visually for spot intensity and drop outs and additionally were subject to rePCR where the PCR output of each device is diluted 1:1000 and inputted into a qPCR protocol to yield a Ct value.

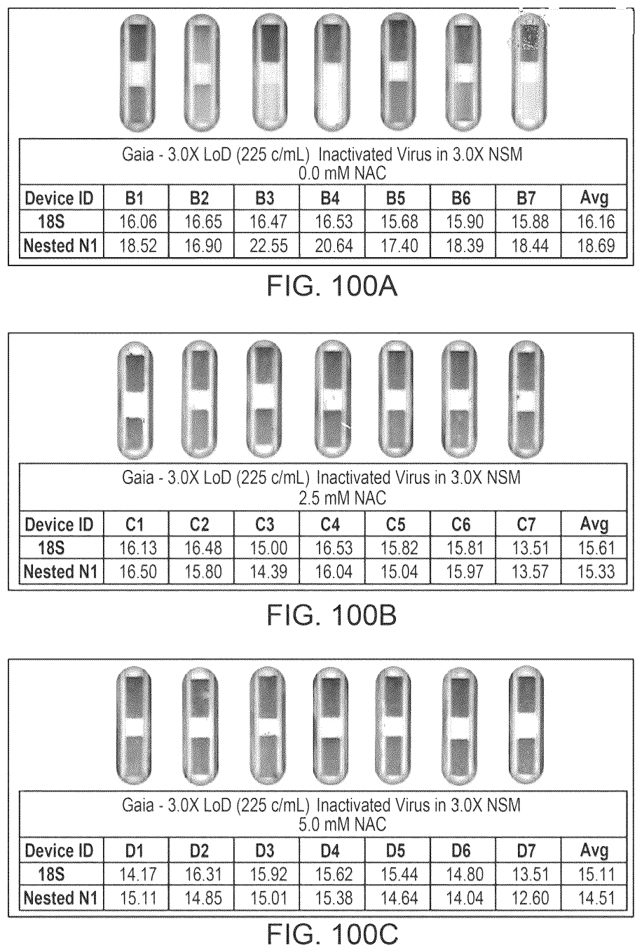

FIG. 100A shows detection of coronavirus genomic RNA in a handheld device without NAC present in the device sample port. Testing of samples at 3× swab equivalents, without reducing agent (NAC), generates variable signal intensity and unfavorably high rePCR Ct values.

FIG. 100B shows detection of coronavirus genomic RNA in a handheld device with lyophilized NAC present in the device sample port at 2.5 mM when rehydrated with the sample. Testing of samples at 3× swab equivalents, with reducing agent (NAC) pellets, generates far less variable signal intensity and relatively low rePCR Ct values when compared to the no NAC POR control.

FIG. 100C shows detection of coronavirus genomic RNA in a handheld device with lyophilized NAC present in the device sample port at 5 mM when rehydrated with the sample. Testing of samples at 3× swab equivalents, with reducing agent (NAC) pellets, generates far less variable signal intensity and further lowers rePCR Ct values when compared to the no NAC POR control and the 2.5 mM NAC condition.

In conclusion the pelletized version of NAC shows drastically increased device performance with 3×NSM at 3×LOD. All devices with NAC present in them completely recovered signal intensity with no dropouts or light spots and drastically lowered rePCR Ct value by up to 4.18 Ct's.

While various embodiments have been described above, it should be understood that they have been presented by way of example only, and not limitation. Where methods and/or schematics described above indicate certain events and/or flow patterns occurring in certain order, the ordering of certain events and/or flow patterns may be modified. While the embodiments have been particularly shown and described, it will be understood that various changes in form and details may be made.

For example, although the amplification modules are generally described herein as performing a thermal cycling operation on the prepared solution, in other embodiment, an amplification module can perform any suitable thermal reaction to amplify nucleic acids within the solution. In some embodiments, any of the amplification modules described herein can perform any suitable type of isothermal amplification process, including, for example, Loop Mediated Isothermal Amplification (LAMP), Nucleic Acid Sequence Based Amplification (NASBA), which can be useful to detect target RNA molecules, Strand Displacement Amplification (SDA), Multiple Displacement Amplification (MDA), Ramification Amplification Method (RAM), or any other type of isothermal process.

For example, any of the sample input modules, sample preparation modules, amplification modules, heater assemblies, and detection modules shown and described herein can be used in any suitable diagnostic device. Such devices can include, for example, a single-use device that can be used in a point-of-care setting and/or in a user's home. Similarly stated, in some embodiments, the device (and any of the other devices shown and described herein) can be configured for use in a decentralized test facility. Further, in some embodiments, any of the sample input modules, sample preparation modules, amplification modules, heater assemblies, and detection modules shown and described herein can be included within a CLIA-waived device and/or can facilitate the operation of a device in accordance with methods that are CLIA waived. Similarly stated, in some embodiments, the sample input modules, the sample preparation modules, the amplification modules, and the detection modules shown and described herein can facilitate operation of a device in a sufficiently simple manner that can produce results with sufficient accuracy to pose a limited likelihood of misuse and/or to pose a limited risk of harm if used improperly. In some embodiments, the sample input modules, the sample preparation modules, the amplification modules, and the detection modules shown and described herein can be used in any of the diagnostic devices shown and described in International Patent Publication No. WO2016/109691, entitled “Devices and Methods for Molecular Diagnostic Testing,” which is incorporated herein by reference in its entirety,” which is incorporated herein by reference in its entirety.

The devices and methods described herein can be used to analyze any suitable type of biological sample, such as a tissue sample (e.g., a blood sample). In some cases, the biological sample comprises a bodily fluid taken from a subject. In some cases, the bodily fluid includes one or more cells comprising nucleic acids. In some cases, the one or more cells comprise one or more microbial cells, including, but not limited to, bacteria, archaebacteria, protists, and fungi. In some cases, the biological sample includes one or more virus particles. In some cases, the biological sample includes one or more microbes that causes a sexually-transmitted disease. A sample may comprise a sample from a subject, such as whole blood; blood products; red blood cells; white blood cells; buffy coat; swabs; urine; sputum; saliva; semen; lymphatic fluid; endolymph; perilymph; gastric juice; bile; mucus; sebum; sweat; tears; vaginal secretion; vomit; feces; breast milk; cerumen; amniotic fluid; cerebrospinal fluid; peritoneal effusions; pleural effusions; biopsy samples; fluid from cysts; synovial fluid; vitreous humor; aqueous humor; bursa fluid; eye washes; eye aspirates; plasma; serum; pulmonary lavage; lung aspirates; animal, including human, tissues, including but not limited to, liver, spleen, kidney, lung, intestine, brain, heart, muscle, pancreas, cell cultures, as well as lysates, extracts, or materials and fractions obtained from the samples described above or any cells and microorganisms and viruses that may be present on or in a sample. A sample may include cells of a primary culture or a cell line. Examples of cell lines include, but are not limited to, 293-T human kidney cells, A2870 human ovary cells, A431 human epithelium, B35 rat neuroblastoma cells, BHK-21 hamster kidney cells, BR293 human breast cells, CHO Chinese hamster ovary cells, CORL23 human lung cells, HeLa cells, or Jurkat cells. The sample may include a homogeneous or mixed population of microbes, including one or more of viruses, bacteria, protists, monerans, chromalveolata, archaea, or fungi. The biological sample can be a urine sample, a vaginal swab, a cervical swab, an anal swab, or a cheek swab. The biological sample can be obtained from a hospital, laboratory, clinical or medical laboratory.

The devices and methods described herein, however, are not limited to performing a molecular diagnostic test on human samples. In some embodiments, any of the devices and methods described herein can be used with veterinary samples, food samples, and/or environmental samples. Examples of environmental sources include, but are not limited to agricultural fields, lakes, rivers, water reservoirs, air vents, walls, roofs, soil samples, plants, and swimming pools. Examples of industrial sources include, but are not limited to clean rooms, hospitals, food processing areas, food production areas, food stuffs, medical laboratories, pharmacies, and pharmaceutical compounding centers. Examples of subjects from which polynucleotides may be isolated include multicellular organisms, such as fish, amphibians, reptiles, birds, and mammals. Examples of mammals include primates (e.g., apes, monkeys, gorillas), rodents (e.g., mice, rats), cows, pigs, sheep, horses, dogs, cats, or rabbits. In some examples, the mammal is a human.

In some embodiments, any of the devices or methods described herein can include a sample buffer (e.g., within a sample preparation module, sample transfer manifold, or reagent module) and/or can mix a sample buffer with the biological sample, or can use the sample buffer as a wash/blocking solution, as described herein. In some cases, the sample buffer can include bovine serum albumin and/or a detergent. In some cases, the sample buffer includes about 0.1% to 5% bovine serum albumin. In some cases, the sample buffer includes about 0.1%, 0.2%, 0.3%, 0.4%, 0.5%, 1%, 1.5%, 2%, 2.5%, 3%, 4%, or 5% bovine serum albumin. In some cases, the sample buffer includes about 0.1% to 20% detergent. In some cases, the sample buffer includes about 1%, 2%, 3%, 4%, 5%, 6%, 7%, 8%, 9%, or 10% detergent. In some cases, the detergent is Tween-20. The choice of sample buffer to be used may depend on the intended method. For example, the choice of sample buffer may different when a wash step will be used to when a wash step is not used. If a wash step will not be used then the sample buffer may be a buffer suitable for lysis and subsequent PCR reactions.

In some embodiments, a sample buffer can include Tris HCL, Tween-80, BSA, Proclin and Antifoam SE-15. In some embodiments, a sample buffer may have a composition of: 50 mM Tris pH 8.4, Tween-80, 2% (w/v), BSA, 0.25% (w/v), Proclin 300 0.03% (w/v), and Antifoam SE-15, 0.002% (v/v) made up in purified water. Tris HCL is a common buffer for PCR. When it is heated during thermocycling, the pH may drop, for example, a Tris buffer with pH of 8.4 at a temperature of 25° C. may drop to a pH of about ˜7.4 when heated to about 95° C. The range of concentrations could be from 0.1 mM to 1 M. The pH range could be from 6 to 10. Any other PCR compatible buffer could be used, for example HEPES. Proclin 300 is a broad spectrum antimicrobial used as a preservative to ensure a long shelf life of the collection media. It could be used from 0.01% (w/v) to 0.1% (w/v). Many other antimicrobials are known in the art and could be used in a sample buffer. In some embodiments, a reagent or wash buffer can include Antifoam SE-15 to reduce foaming during manufacturing and fluidic movement through the device. It could be used from 0.001% (v/v) to 1% (v/v). Any other antifoam agent could also be used, for example, Antifoam 204, Antifoam A, Antifoam B, Antifoam C, or Antifoam Y-30.

In some embodiments, any of the amplification modules described can be configured to conduct a “rapid” PCR (e.g., completing at least 30 cycles in less than about 10 minutes), and rapid production of an output signal (e.g., via a detection module). Similarly stated, the amplification modules described herein can be configured to process volumes, to have dimensional sizes and/or be constructed from materials that facilitates a rapid PCR or amplification in less than about 10 minutes, less than about 9 minutes, less than about 8 minutes, less than about 7 minutes, less than about 6 minutes, or any range therebetween, as described herein.

In some embodiments, the capture probes can be formulated, designed or engineered to have a relatively high melting temperature (Tm) value (e.g., approximately 67° C.). In other embodiments, the capture probes can have a melting temperature (Tm) value that ranges from 35° C. to 85° C., 60° C. to 85° C., 60° C. to 75° C., 65° C. to 70° C., or 66° C. to 68° C. One advantage of capture probes having a high Tm value is that the flow cell can be heated to a wide range of temperatures during operation without causing the capture probe to release the target amplicon.

In some embodiments, the capture probes are designed against sequences from Neisseria gonorrhoeae, Chlamydia trachomatis, Trichomonas vaginalis, Neisseria subflava and a negative control sequence such as sequence from Bacillus atrophaeus or random bases.

Some embodiments described herein relate to a computer storage product with a non-transitory computer-readable medium (also can be referred to as a non-transitory processor-readable medium) having instructions or computer code thereon for performing various computer-implemented operations. The computer-readable medium (or processor-readable medium) is non-transitory in the sense that it does not include transitory propagating signals per se (e.g., a propagating electromagnetic wave carrying information on a transmission medium such as space or a cable). The media and computer code (also can be referred to as code) may be those designed and constructed for the specific purpose or purposes. Examples of non-transitory computer-readable media include, but are not limited to: magnetic storage media such as hard disks, floppy disks, and magnetic tape; optical storage media such as Compact Disc/Digital Video Discs (CD/DVDs), Compact Disc-Read Only Memories (CD-ROMs), and holographic devices; magneto-optical storage media such as optical disks; carrier wave signal processing modules; and hardware devices that are specially configured to store and execute program code, such as Application-Specific Integrated Circuits (ASICs), Programmable Logic Devices (PLDs), Read-Only Memory (ROM) and Random-Access Memory (RAM) devices.

Examples of computer code include, but are not limited to, micro-code or microinstructions, machine instructions, such as produced by a compiler, code used to produce a web service, and files containing higher-level instructions that are executed by a computer using an interpreter. For example, embodiments may be implemented using imperative programming languages (e.g., C, Fortran, etc.), functional programming languages (Haskell, Erlang, etc.), logical programming languages (e.g., Prolog), object-oriented programming languages (e.g., Java, C++, etc.) or other suitable programming languages and/or development tools. Additional examples of computer code include, but are not limited to, control signals, encrypted code, and compressed code.

The processor included within a control module (and any of the processors and/or controllers described herein) can be any processor configured to, for example, write data into and read data from the memory of the controller, and execute the instructions and/or methods stored within the memory. Furthermore, the processor can be configured to control operation of the other modules within the controller (e.g., the temperature feedback module and the flow module). Specifically, the processor can receive a signal including temperature data, current measurements or the like and determine an amount of power and/or current to be supplied to each heater assembly, the desired timing and sequence of the piston pulses and the like. For example, in some embodiments, the controller can be an 8-bit PIC microcontroller, which will control the power delivered to various heating assemblies and components within the amplification module 4600. This microcontroller can also contain code for and/or be configured to minimize the instantaneous power requirements on the power source.

In other embodiments, any of the processors described herein can be, for example, an application-specific integrated circuit (ASIC) or a combination of ASICs, which are designed to perform one or more specific functions. In yet other embodiments, the microprocessor can be an analog or digital circuit, or a combination of multiple circuits.

Any of the memory devices described herein can be any suitable device such as, for example, a read only memory (ROM) component, a random access memory (RAM) component, electronically programmable read only memory (EPROM), erasable electronically programmable read only memory (EEPROM), registers, cache memory, and/or flash memory. Any of the modules (the pressure feedback module and the position feedback module) can be implemented by the processor and/or stored within the memory.

Although various embodiments have been described as having particular features and/or combinations of components, other embodiments are possible having a combination of any features and/or components from any of embodiments as discussed above.

Any of the devices and methods described herein can be utilized to detect the presence or absence of nucleic acids associated with one or more viruses in a biological sample. Non-limiting examples of viruses include the herpes virus (e.g., human cytomegalomous virus (HCMV), herpes simplex virus 1 (HSV-1), herpes simplex virus 2 (HSV-2), varicella zoster virus (VZV), Epstein-Barr virus), influenza A virus and Hepatitis C virus (HCV) or a picomavirus such as Coxsackievirus B3 (CVB3). Other viruses may include, but are not limited to, the hepatitis B virus, HIV, poxvirus, hepadavirus, retrovirus, and RNA viruses such as flavivirus, togavirus, coronavirus, Hepatitis D virus, orthomyxovirus, paramyxovirus, rhabdovirus, bunyavirus, filo virus, Adenovirus, Human herpesvirus, type 8, Human papillomavirus, BK virus, JC virus, Smallpox, Hepatitis B virus, Human bocavirus, Parvovirus B 19, Human astrovirus, Norwalk virus, coxsackievirus, hepatitis A virus, poliovirus, rhinovirus, Severe acute respiratory syndrome virus, Hepatitis C virus, yellow fever virus, dengue virus, West Nile virus, Rubella virus, Hepatitis E virus, and Human immunodeficiency virus (HIV). In some embodiments, the virus is an enveloped virus. Examples of such enveloped viruses include, but are not limited to, viruses that are members of the hepadnavirus family, herpesvirus family, iridovirus family, poxvirus family, flavivirus family, togavirus family, retrovirus family, coronavirus family, filovirus family, rhabdovirus family, bunyavirus family, orthomyxovirus family, paramyxovirus family, and arenavirus family. Other examples include, but are not limited to, Hepadnavirus hepatitis B virus (HBV), woodchuck hepatitis virus, ground squirrel (Hepadnaviridae) hepatitis virus, duck hepatitis B virus, heron hepatitis B virus, Herpesvirus herpes simplex virus (HSV) types 1 and 2, varicellazoster virus, cytomegalovirus (CMV), human cytomegalovirus (HCMV), mouse cytomegalovirus (MCMV), guinea pig cytomegalovirus (GPCMV), Epstein-Barr virus (EBV), human herpes virus 6 (HHV variants A and B), human herpes virus 7 (HHV-7), human herpes virus 8 (HHV-8), Kaposi's sarcoma-associated herpes virus (KSHV), B virus Poxvirus vaccinia virus, variola virus, smallpox virus, monkeypox virus, cowpox virus, camelpox virus, ectromelia virus, mousepox virus, rabbitpox viruses, raccoon pox viruses, molluscum contagiosum virus, orf virus, milker's nodes virus, bovin papullar stomatitis virus, sheeppox virus, goatpox virus, lumpy skin disease virus, fowlpox virus, canarypox virus, pigeonpox virus, sparrowpox virus, myxoma virus, hare fibroma virus, rabbit fibroma virus, squirrel fibroma viruses, swinepox virus, tanapox virus, Yabapox virus, Flavivirus dengue virus, hepatitis C virus (HCV), GB hepatitis viruses (GBV-A, GBV-B and GBV-C), West Nile virus, yellow fever virus, St. Louis encephalitis virus, Japanese encephalitis virus, Powassan virus, tick-borne encephalitis virus, Kyasanur Forest disease virus, Togavirus, Venezuelan equine encephalitis (VEE) virus, chikungunya virus, Ross River virus, Mayaro virus, Sindbis virus, rubella virus, Retrovirus human immunodeficiency virus (HIV) types 1 and 2, human T cell leukemia virus (HTLV) types 1, 2, and 5, mouse mammary tumor virus (MMTV), Rous sarcoma virus (RSV), lentiviruses, Coronavirus, severe acute respiratory syndrome (SARS) virus, middle east respiratory syndrome (MERS), Filovirus Ebola virus, Marburg virus, Metapneumoviruses (MPV) such as human metapneumovirus (HMPV), Rhabdovirus rabies virus, vesicular stomatitis virus, Bunyavirus, Crimean-Congo hemorrhagic fever virus, Rift Valley fever virus, La Crosse virus, Hantaan virus, Orthomyxovirus, influenza virus (types A, B, and C), Paramyxovirus, parainfluenza virus (PIV types 1, 2 and 3), respiratory syncytial virus (types A and B), measles virus, mumps virus, Arenavirus, lymphocytic choriomeningitis virus, Junin virus, Machupo virus, Guanarito virus, Lassa virus, Ampari virus, Flexal virus, Ippy virus, Mobala virus, Mopeia virus, Latino virus, Parana virus, Pichinde virus, Punta torn virus (PTV), Tacaribe virus and Tamiami virus. In some embodiments, the virus is a non-enveloped virus, examples of which include, but are not limited to, viruses that are members of the parvovirus family, circovirus family, polyoma virus family, papillomavirus family, adenovirus family, iridovirus family, reovirus family, birnavirus family, calicivirus family, and picornavirus family. Specific examples include, but are not limited to, canine parvovirus, parvovirus B19, porcine circovirus type 1 and 2, BFDV (Beak and Feather Disease virus, chicken anaemia virus, Polyomavirus, simian virus 40 (SV40), JC virus, BK virus, Budgerigar fledgling disease virus, human papillomavirus, bovine papillomavirus (BPV) type 1, cotton tail rabbit papillomavirus, human adenovirus (HAdV-A, HAdV-B, HAdV-C, HAdV-D, HAdV-E, and HAdV-F), fowl adenovirus A, bovine adenovirus D, frog adenovirus, Reovirus, human orbivirus, human coltivirus, mammalian orthoreovirus, bluetongue virus, rotavirus A, rotaviruses (groups B to G), Colorado tick fever virus, aquareovirus A, cypovirus 1, Fiji disease virus, rice dwarf virus, rice ragged stunt virus, idnoreovirus 1, mycoreovirus 1, Bimavirus, bursal disease virus, pancreatic necrosis virus, Calicivirus, swine vesicular exanthema virus, rabbit hemorrhagic disease virus, Norwalk virus, Sapporo virus, Picornavirus, human polioviruses (1-3), human coxsackieviruses Al-22, 24 (CAl-22 and CA24, CA23 (echovirus 9)), human coxsackieviruses (Bl-6 (CBl-6)), human echoviruses 1-7, 9, 11-27, 29-33, vilyuish virus, simian enteroviruses 1-18 (SEVI-18), porcine enteroviruses 1-11 (PEVl-11), bovine enteroviruses 1-2 (BEVI-2), hepatitis A virus, rhinoviruses, hepatoviruses, cardio viruses, aphthoviruses and echoviruses. The virus may be phage. Examples of phages include, but are not limited to T4, TS, λ phage, T7 phage, G4, Pl, φ6, Thermoproteus tenax virus 1, M13, MS2, Qβ, φ X174, Φ29, PZA, Φ15, BS32, B103, M2Y (M2), Nf, GA-I, FWLBc1, FWLBc2, FWLLm3, B4. The reference database may comprise sequences for phage that are pathogenic, protective, or both. In some cases, the virus is selected from a member of the Flaviviridae family (e.g., a member of the Flavivirus, Pestivirus, and Hepacivirus genera), which includes the hepatitis C virus, Yellow fever virus; Tick-borne viruses, such as the Gadgets Gully virus, Kadam virus, Kyasanur Forest disease virus, Langat virus, Omsk hemorrhagic fever virus, Powassan virus, Royal Farm virus, Karshi virus, tick-borne encephalitis virus, Neudoerfl virus, Sofjin virus, Louping ill virus and the Negishi virus; seabird tick-borne viruses, such as the Meaban virus, Saumarez Reef virus, and the Tyuleniy virus; mosquito-borne viruses, such as the Arna virus, dengue virus, Kedougou virus, Cacipacore virus, Koutango virus, Japanese encephalitis virus, Murray Valley encephalitis virus, St. Louis encephalitis virus, Usutu virus, West Nile virus, Yaounde virus, Kokobera virus, Bagaza virus, Ilheus virus, Israel turkey meningoencephalo-myelitis virus, Ntaya virus, Tembusu virus, Zika virus, Banzi virus, Bouboui virus, Edge Hill virus, Jugra virus, Saboya virus, Sepik virus, Uganda S virus, Wesselsbron virus, yellow fever virus; and viruses with no known arthropod vector, such as the Entebbe bat virus, Yokose virus, Apoi virus, Cowbone Ridge virus, Jutiapa virus, Modoc virus, Sal Vieja virus, San Perlita virus, Bukalasa bat virus, Carey Island virus, Dakar bat virus, Montana myotis leukoencephalitis virus, Phnom Penh bat virus, Rio Bravo virus, Tamana bat virus, and the Cell fusing agent virus. In some cases, the virus is selected from a member of the Arenaviridae family, which includes the Ippy virus, Lassa virus (e.g., the Josiah, LP, or GA391 strain), lymphocytic choriomeningitis virus (LCMV), Mobala virus, Mopeia virus, Amapari virus, Flexal virus, Guanarito virus, Junin virus, Latino virus, Machupo virus, Oliveros virus, Parana virus, Pichinde virus, Pirital virus, Sabia virus, Tacaribe virus, Tamiami virus, Whitewater Arroyo virus, Chapare virus, and Lujo virus. In some cases, the virus is selected from a member of the Bunyaviridae family (e.g., a member of the Hantavirus, Nairovirus, Orthobunyavirus, and Phlebovirus genera), which includes the Hantaan virus, Sin Nombre virus, Dugbe virus, Bunyamwera virus, Rift Valley fever virus, La Crosse virus, Punta Toro virus (PTV), California encephalitis virus, and Crimean-Congo hemorrhagic fever (CCHF) virus. In some cases, the virus is selected from a member of the Filoviridae family, which includes the Ebola virus (e.g., the Zaire, Sudan, Ivory Coast, Reston, and Uganda strains) and the Marburg virus (e.g., the Angola, Ci67, Musoke, Popp, Ravn and Lake Victoria strains); a member of the Togaviridae family (e.g., a member of the Alphavirus genus), which includes the Venezuelan equine encephalitis virus (VEE), Eastern equine encephalitis virus (EEE), Western equine encephalitis virus (WEE), Sindbis virus, rubella virus, Semliki Forest virus, Ross River virus, Barmah Forest virus, O' nyong'nyong virus, and the chikungunya virus; a member of the Poxyiridae family (e.g., a member of the Orthopoxvirus genus), which includes the smallpox virus, monkeypox virus, and vaccinia virus; a member of the Herpesviridae family, which includes the herpes simplex virus (HSV; types 1, 2, and 6), human herpes virus (e.g., types 7 and 8), cytomegalovirus (CMV), Epstein-Barr virus (EBV), Varicella-Zoster virus, and Kaposi's sarcoma associated-herpesvirus (KSHV); a member of the Orthomyxoviridae family, which includes the influenza virus (A, B, and C), such as the H5N1 avian influenza virus or HINI swine flu; a member of the Coronaviridae family, which includes the severe acute respiratory syndrome (SARS) virus; a member of the Rhabdoviridae family, which includes the rabies virus and vesicular stomatitis virus (VSV); a member of the Paramyxoviridae family, which includes the human respiratory syncytial virus (RSV), Newcastle disease virus, hendravirus, nipahvirus, measles virus, rinderpest virus, canine distemper virus, Sendai virus, human parainfluenza virus (e.g., 1, 2, 3, and 4), rhinovirus, and mumps virus; a member of the Picomaviridae family, which includes the poliovirus, human enterovirus (A, B, C, and D), hepatitis A virus, and the coxsackievirus; a member of the Hepadnaviridae family, which includes the hepatitis B virus; a member of the Papillamoviridae family, which includes the human papilloma virus; a member of the Parvoviridae family, which includes the adeno-associated virus; a member of the Astroviridae family, which includes the astrovirus; a member of the Polyomaviridae family, which includes the JC virus, BK virus, and SV40 virus; a member of the Calciviridae family, which includes the Norwalk virus; a member of the Reoviridae family, which includes the rotavirus; and a member of the Retroviridae family, which includes the human immunodeficiency virus (HIV; e.g., types I and 2), and human T-lymphotropic virus Types I and II (HTLV-1 and HTLV-2, respectively).

Any of the devices and methods described herein can be utilized to detect the presence or absence of nucleic acids associated with one or more fungi in a biological sample. Examples of infectious fungal agents include, without limitation Aspergillus, Blastomyces, Coccidioides, Cryptococcus, Histoplasma, Paracoccidioides, Sporothrix, and at least three genera of Zygomycetes. The above fungi, as well as many other fungi, can cause disease in pets and companion animals. The present teaching is inclusive of substrates that contact animals directly or indirectly. Examples of organisms that cause disease in animals include Malassezia furfur, Epidermophyton floccosur, Trichophyton mentagrophytes, Trichophyton rubrum, Trichophyton tonsurans, Trichophyton equinum, Dermatophilus congolensis, Microsporum canis, Microsporu audouinii, Microsporum gypseum, Malassezia ovale, Pseudallescheria, Scopulariopsis, Scedosporium, and Candida albicans. Further examples of fungal infectious agent include, but are not limited to, Aspergillus, Blastomyces dermatitidis, Candida, Coccidioides immitis, Cryptococcus neoformans, Histoplasma capsulatum var. capsulatum, Paracoccidioides brasiliensis, Sporothrix schenckii, Zygomycetes spp., Absidia corymbifera, Rhizomucor pusillus, or Rhizopus arrhizus.

Any of the devices and methods described herein can be utilized to detect the presence or absence of nucleic acids associated with one or more parasites in a biological sample. Non-limiting examples of parasites include Plasmodium, Leishmania, Babesia, Treponema, Borrelia, Trypanosoma, Toxoplasma gondii, Plasmodium falciparum, P. vivax, P. ovale, P. malariae, Trypanosoma spp., or Legionella spp. In some cases, the parasite is Trichomonas vaginalis.

In various embodiments, the limited of detection of the methods and devices disclosed herein is 125 genome copies/milliliter (gc/mL), 250 gc/mL, 500 gc/mL, 750 gc/mL, or 1000 gc/mL.

In various embodiments, the sensitivity of the methods and devices disclosed herein is at least 90%, at least 91%, at least 91%, at least 92%, at least 93%, at least 94%, at least 95%, at least 96%, at least 97%, at least 98%, or at least 99%.

In various embodiments, the specificity of the methods and devices disclosed herein is at least 90%, at least 91%, at least 91%, at least 92%, at least 93%, at least 94%, at least 95%, at least 96%, at least 97%, at least 98%, or at least 99%.

In various embodiments, the sensitivity and specificity of the methods and devices disclosed herein are at least 90% and at least 90%, respectively; at least 90% and at least 91%, respectively; at least 90% and at least 92%, respectively; at least 90% and at least 93%, respectively; at least 90% and at least 94%, respectively; at least 90% and at least 95%, respectively; at least 90% and at least 96%, respectively; at least 97% and at least 98%, respectively; or at least 90% and at least 99%, respectively.

In various embodiments, the sensitivity and specificity of the methods and devices disclosed herein are at least 91% and at least 90%, respectively; at least 91% and at least 91%, respectively; at least 91% and at least 92%, respectively; at least 91% and at least 93%, respectively; at least 91% and at least 94%, respectively; at least 91% and at least 95%, respectively; at least 91% and at least 96%, respectively; at least 97% and at least 98%, respectively; or at least 91% and at least 99%, respectively.

In various embodiments, the sensitivity and specificity of the methods and devices disclosed herein are at least 92% and at least 90%, respectively; at least 92% and at least 91%, respectively; at least 92% and at least 92%, respectively; at least 92% and at least 93%, respectively; at least 92% and at least 94%, respectively; at least 92% and at least 95%, respectively; at least 92% and at least 96%, respectively; at least 97% and at least 98%, respectively; or at least 92% and at least 99%, respectively.

In various embodiments, the sensitivity and specificity of the methods and devices disclosed herein are at least 93% and at least 90%, respectively; at least 93% and at least 91%, respectively; at least 93% and at least 92%, respectively; at least 93% and at least 93%, respectively; at least 93% and at least 94%, respectively; at least 93% and at least 95%, respectively; at least 93% and at least 96%, respectively; at least 97% and at least 98%, respectively; or at least 93% and at least 99%, respectively.

In various embodiments, the sensitivity and specificity of the methods and devices disclosed herein are at least 94% and at least 90%, respectively; at least 94% and at least 91%, respectively; at least 94% and at least 92%, respectively; at least 94% and at least 93%, respectively; at least 94% and at least 94%, respectively; at least 94% and at least 95%, respectively; at least 94% and at least 96%, respectively; at least 97% and at least 98%, respectively; or at least 94% and at least 99%, respectively.

In various embodiments, the sensitivity and specificity of the methods and devices disclosed herein are at least 95% and at least 90%, respectively; at least 95% and at least 91%, respectively; at least 95% and at least 92%, respectively; at least 95% and at least 93%, respectively; at least 95% and at least 94%, respectively; at least 95% and at least 95%, respectively; at least 95% and at least 96%, respectively; at least 97% and at least 98%, respectively; or at least 95% and at least 99%, respectively.

In various embodiments, the sensitivity and specificity of the methods and devices disclosed herein are at least 96% and at least 90%, respectively; at least 96% and at least 91%, respectively; at least 96% and at least 92%, respectively; at least 96% and at least 93%, respectively; at least 96% and at least 94%, respectively; at least 96% and at least 95%, respectively; at least 96% and at least 96%, respectively; at least 97% and at least 98%, respectively; or at least 96% and at least 99%, respectively.

In various embodiments, the sensitivity and specificity of the methods and devices disclosed herein are at least 97% and at least 90%, respectively; at least 97% and at least 91%, respectively; at least 97% and at least 92%, respectively; at least 97% and at least 93%, respectively; at least 97% and at least 94%, respectively; at least 97% and at least 95%, respectively; at least 97% and at least 96%, respectively; at least 97% and at least 98%, respectively; or at least 97% and at least 99%, respectively.

In various embodiments, the sensitivity and specificity of the methods and devices disclosed herein are at least 98% and at least 90%, respectively; at least 98% and at least 91%, respectively; at least 98% and at least 92%, respectively; at least 98% and at least 93%, respectively; at least 98% and at least 94%, respectively; at least 98% and at least 95%, respectively; at least 98% and at least 96%, respectively; at least 97% and at least 98%, respectively; or at least 98% and at least 99%, respectively.

In various embodiments, the sensitivity and specificity of the methods and devices disclosed herein are at least 99% and at least 90%, respectively; at least 99% and at least 91%, respectively; at least 99% and at least 92%, respectively; at least 99% and at least 93%, respectively; at least 99% and at least 94%, respectively; at least 99% and at least 95%, respectively; at least 99% and at least 96%, respectively; at least 97% and at least 98%, respectively; or at least 99% and at least 99%, respectively.

Additional embodiments and details are included the Appendix, which is attached to this specification.

What is claimed is:

1. A method of detecting a target pathogen in a plurality of biological samples using a stand-alone molecular diagnostic test device, comprising:
combining each of the plurality of biological samples to form a combined input sample;
conveying the combined input sample into the stand-alone molecular diagnostic test device via an input opening; and
actuating the stand-alone molecular diagnostic test device to cause the stand-alone molecular diagnostic test device to:
heat the combined input sample within a sample preparation module to produce a complementary DNA (cDNA) associated with the target pathogen;
heat the combined input sample within an amplification module to amplify the cDNA within the combined input sample to produce an output solution containing a target amplicon; and
react within a detection module each of A) the output solution and B) a reagent formulated to produce a signal that indicates a presence of the target amplicon within the output solution, the detection module including one or more probes specific to a polynucleotide sequence of the target pathogen;
wherein the sample preparation module, the amplification module, and the detection module are integrated within the stand-alone molecular diagnostic test device.

2. The method of claim 1, wherein the target pathogen is a virus.

3. The method of claim 2, wherein the virus is a coronavirus, optionally a betacoronavirus.

4. The method of claim 2, wherein the virus is a coronavirus, optionally sudden acute respiratory virus coronavirus 2 (SARS-CoV-2).

5. The method of claim 2, wherein each of the plurality of biological samples is from a swab.

6. The method of claim 5, wherein each of the plurality of biological samples is collected from one of a nasal swab, a mid-turbinate swab, a nasopharyngeal swab, or an oropharyngeal swab.

7. The method of any one of claims 1-6, wherein the plurality of biological samples includes between two biological samples and twenty biological samples.

8. The method of claim 7, wherein the plurality of biological samples includes between three biological samples and ten biological samples.

9. The method of claim 8, wherein the plurality of biological samples includes between four biological samples and eight biological samples.

10. The method of claim 9, wherein the plurality of biological samples includes five biological samples.

11. The method of claim 5, wherein:
the combining includes conveying each of the biological samples into a sample tube containing a buffer or a transport media.

12. The method of claim 5, wherein the combining includes:
conveying each of the biological samples into a corresponding sample tube containing a buffer or a transport media; and
mixing the contents of each of the sample tubes.

13. The method of any one of claims 11-12, wherein the reducing agent is applied via a dry reagent coating process to a surface of the sample preparation module.

14. The method of any one of claim 11 or 12, wherein at least one of the sample tube or the sample preparation module includes a reducing agent.

15. The method of claim 14, wherein the reducing agent is selected from the group consisting of lithium, sodium, potassium, aluminum amalgam, lithium aluminum hydride, sodium borohydride, sodium cyanoborohydride, lithium tri-butoxy aluminum hydride, dibutoxy aluminum hydride, lithium triethyl borohydride, tris(2-carboxyethyl)phosphine (TCEP), dithiothreitol (DTT), mercaptoethanol, cysteine, thioglycerol, thioglycolic acid, cysteamine, glutathione, N-acetylcysteine (NAC), β-mercaptoethanol, sodium dodecyl sulfate (SDS), sodium sulfite, ammonium sulfamate, sodium bisulfite, dithionite metabisulfite sulfur dioxide, vitamin C, ascorbic acid, and dimethylsulfoxide (DMSO).

16. The method of claim 15, wherein the reducing agent is N-acetylcysteine (NAC).

17. The method of claim 16, wherein the N-acetylcysteine is N-acetyl-L-cysteine.

18. The method of claim 14, wherein the reducing agent is a pellet stored within the sample preparation module.

19. The method of claim 18, wherein the pellet comprises lyophilized particles.

20. The method of claim 19, wherein the pellet comprises enzymes.

21. The method of claim 19, wherein the pellet comprises nucleic acids.

22. The method of claim 19, wherein the pellet comprises NAC and an oligonucleotide.

23. The method of claim 19, wherein the pellet comprises NAC, an oligonucleotide, and a buffer, and optionally a surfactant.

24. The method of any claim 11 or 12, wherein the cDNA is amplified within the amplification module by polymerase chain reaction (PCR).

25. The method of claim 24, wherein:
the amplification module comprises a serpentine flow channel and a heater coupled to the serpentine flow channel; and
the actuating the stand-alone molecular diagnostic test device causes the stand-alone molecular diagnostic test device to produce a flow of the combined input sample within the amplification module to amplify the cDNA.

26. The method of any one of claim 11 or 12, further comprising:
storing the stand-alone molecular diagnostic test device for at least six months.

27. The method of any one of claim 11 or 12, further comprising:
reading a result associated with the signal.

28. The method of claim 27, further comprising:
discarding, after the reading, the stand-alone molecular diagnostic test device.

29. The method of claim 27, further comprising:
returning, after the reading, the stand-alone molecular diagnostic test device to a centralized facility.

30. The method of any one of claim 11 or 12, wherein the signal is produced within about 30 minutes after the actuating the stand-alone molecular diagnostic test device.

31. A kit for detecting a target RNA virus in a plurality of biological samples, the kit comprising:
a sample tube containing a buffer, the sample tube configured to receive the plurality of biological samples to form a combined input sample;
a sample transfer device; and
a stand-alone molecular diagnostic test device, the stand-alone molecular diagnostic test device comprising:
a housing defining an input opening through which the combined input sample can be conveyed from the sample tube into the stand-alone molecular diagnostic test device using the sample transfer device;
a sample preparation module within the housing, the sample preparation module configured to heat the combined input sample to produce complementary DNA (cDNA) associated with the RNA virus thereby producing an amplification solution;
an amplification module within the housing, the amplification module defining a reaction volume configured to receive the amplification solution and amplify the cDNA molecule within the amplification solution to produce an output; and
a detection module within the housing, the detection module configured to receive the output from the amplification module, the detection module including one or more probes specific to a polynucleotide sequence of the RNA virus, the one or more probes designed to facilitate production of a signal indicating the presence of the RNA virus in the combined input sample.

32. The kit of claim 31, wherein the reducing agent is applied via a dry reagent coating process to a surface of the sample preparation module.

33. The kit of claim 31, further comprising:
a reducing agent, the reducing agent stored within one of the sample tube or the sample preparation module.

34. The kit of claim 33, wherein the reducing agent is selected from the group consisting of lithium, sodium, potassium, aluminum amalgam, lithium aluminum hydride, sodium borohydride, sodium cyanoborohydride, lithium tri-butoxy aluminum hydride, dibutoxy aluminum hydride, lithium triethyl borohydride, tris(2-carboxyethyl)phosphine (TCEP), dithiothreitol (DTT), mercaptoethanol, cysteine, thioglycerol, thioglycolic acid, cysteamine, glutathione, N-acetylcysteine (NAC), β-mercaptoethanol, sodium dodecyl sulfate (SDS), sodium sulfite, ammonium sulfamate, sodium bisulfite, dithionite metabisulfite sulfur dioxide, vitamin C, ascorbic acid, and dimethylsulfoxide (DMSO).

35. The kit of claim 34, wherein the reducing agent is N-acetylcysteine (NAC).

36. The kit of claim 35, wherein the N-acetylcysteine is N-acetyl-L-cysteine.

37. The kit of claim 35, wherein the reducing agent is a pellet stored within the sample preparation module.

38. The kit of claim 37, wherein the pellet comprises lyophilized particles.

39. The kit of claim 38, wherein the pellet comprises enzymes.

40. The kit of claim 38, wherein the pellet comprises nucleic acids.

41. The kit of claim 38, wherein the pellet comprises NAC and an oligonucleotide.

42. The kit of claim 38, wherein the pellet comprises NAC, an oligonucleotide, and a buffer.

43. A method of detecting a coronavirus from a plurality of biological samples using a stand-alone molecular diagnostic test device, comprising:
combining a biological sample from each of the plurality of patients to form a combined input sample;
conveying the input sample to a reverse transcription module within a housing of the stand-alone molecular diagnostic test device;
heating the input sample within the reverse transcription module to produce a target DNA molecule associated with the coronavirus;
conveying the input sample from the reverse transcription module to an amplification module within the housing, the amplification module defining a reaction volume;
heating the input sample within at least a portion of the reaction volume via the heater to amplify the target DNA molecule within the input sample thereby producing an output solution; and
conveying into a detection module each of A) the output solution and B) a reagent formulated to produce a signal that indicates a presence of the coronavirus within the output solution, the detection module including one or more probes specific to a coronavirus polynucleotide sequence.

44. The method of claim 43, further comprising:
detecting a magnitude of the signal; and
producing an output providing a range of the plurality of patients having the coronavirus.

45. The method of claim 43, wherein the plurality of patients is between five and twenty.

46. The method of claim 43, wherein the stand-alone molecular diagnostic test device is a first stand-alone molecular diagnostic test device, the method further comprising:
testing a patient from the plurality of patients using a second stand-alone molecular diagnostic test device when the signal indicates the presence of the coronavirus.

47. A stand-alone molecular diagnostic test device, comprising:
a housing;
a reverse transcription module within the housing, the reverse transcription module configured to heat a biological sample to produce a target cDNA molecule associated with an RNA virus thereby producing an amplification solution;
an amplification module within the housing, the amplification module defining a reaction volume configured to receive the amplification solution and amplify the target cDNA molecule within the amplification solution to produce an output; and
a detection module within the housing, the detection module configured to receive the output from the amplification module, the detection module including one or more probes specific to a polynucleotide sequence of the RNA virus, the one or more probes designed to facilitate production of a signal indicating the presence of the RNA virus in the biological sample.

48. The stand-alone molecular diagnostic test device of claim 47, wherein the RNA virus is influenza, optionally influenza A or influenza B.

49. The stand-alone molecular diagnostic test device of claim 47, wherein the RNA virus is a paramyxovirus, optionally respiratory syncytial virus.

50. The stand-alone molecular diagnostic test device of claim 47, wherein the RNA virus is a coronavirus, optionally a betacoronavirus.

51. The stand-alone molecular diagnostic test device of claim 47, wherein the RNA virus is a coronavirus, optionally SARS-CoV-2.

52. The stand-alone molecular diagnostic test device of claim 47, wherein:
the one or more probes includes a first probe and a second probe, the first probe specific to a first polynucleotide sequence of one of an influenza virus, a paramyxovirus, or a coronavirus, the second probe specific to a second polynucleotide sequence of another of the influenza virus, the paramyxovirus, or the coronavirus;
the detection module includes a first detection surface to which the first probe is adhered and a second detection surface to which the second probe is adhered; and
a first signal is produced from the first detection surface when the first polynucleotide sequence is present and a second signal is produced from the second detection surface when the second polynucleotide sequence is present.

53. The stand-alone molecular diagnostic test device of claim 52, further comprising:
a reagent module within the housing, the reagent module including a reagent formulated to produce a first color product from the first detection surface and a second color product from the second detection surface when the reagent is conveyed into the detection module.

54. The stand-alone molecular diagnostic test device of claim 53, wherein the reverse transcription module, the amplification module, the detection module, and the reagent module are integrated within the housing, the detection module being positioned within the housing such that the first color product and the second color product are viewable via a detection opening of the housing.

55. The stand-alone molecular diagnostic test device of claim 54, further comprising:
an electronic system including a digital read module implemented in at least one of a memory or a processing device, the digital read module configured to: A) detect the presence of the first signal and the second signal and B) produce an electronic output associated with the presence of at least one of the first signal and the second signal.

56. A stand-alone molecular diagnostic test device, comprising:
a housing;
a reverse transcription module within the housing, the reverse transcription module configured to heat a biological sample to produce a target cDNA molecule associated with a coronavirus thereby producing an amplification solution;
an amplification module within the housing, the amplification module defining a reaction volume configured to receive the amplification solution and amplify the target cDNA molecule within the amplification solution to produce an output; and
a detection module within the housing, the detection module configured to receive the output from the amplification module, the detection module including one or more probes specific to a coronavirus polynucleotide sequence, the one or more probes designed to facilitate production of a signal indicating the presence of the coronavirus in the biological sample.

57. A method of detecting a coronavirus using a disposable molecular diagnostic test device, comprising:
conveying a biological sample to a reverse transcription module within a housing of the disposable molecular diagnostic test device;
heating the biological sample within the reverse transcription module to produce a target DNA molecule associated with the coronavirus;
conveying the biological sample from the reverse transcription module to an amplification module within the housing, the amplification module defining a reaction volume;
heating the biological sample within at least a portion of the reaction volume via a heater to amplify the target DNA molecule within the input sample thereby producing an output solution; and
conveying into a detection module each of A) the output solution and B) a reagent formulated to produce a signal that indicates a presence of the coronavirus within the output solution, the detection module including one or more probes specific to a coronavirus polynucleotide sequence, the one or more probes adhered to a detection surface within the detection module.

58. The method of claim 57, wherein the heating the biological sample within the reverse transcription module includes transcribing the genome of the coronavirus.

59. The method of claim 57, wherein the biological sample is from a nasal swab.

60. The method of claim 59, wherein the nasal swab is one of mid-turbinate or nasopharyngeal.

61. A probe set for detecting the presence of at least any of an influenza virus, a paramyxovirus, or a coronavirus, the probe set comprising:
a first probe specific to a first polynucleotide sequence of one of the influenza virus, the paramyxovirus, or the coronavirus; and
a second probe specific to a second polynucleotide sequence of another of the influenza virus, the paramyxovirus, or the coronavirus,
wherein each of the first probe and the second probe has a melting temperature within about 5 degrees C. of a reference melting temperature.

62. The probe set of claim 61, wherein the first probe is specific to the first polynucleotide sequence of the influenza virus and the second probe is specific to the second polynucleotide sequence of the paramyxovirus, the probe set further comprising:
a third probe specific to a third polynucleotide sequence of the coronavirus.


SEQUENCE LISTING (9 sequences)

SEQ ID NO: 1 - DNA, 20 bp, Betacoronavirus Severe acute respiratory syndrome coronavirus 2
gaccccaaaatcagcgaaat

SEQ ID NO: 2 - DNA, 24 bp, Betacoronavirus Severe acute respiratory syndrome coronavirus 2
  misc_feature (1)..(1): biotinylated
tctggttactgccagttgaatctg

SEQ ID NO: 3 - DNA, 20 bp, Betacoronavirus Severe acute respiratory syndrome coronavirus 2
ttacaaacattggccgcaaa

SEQ ID NO: 4 - DNA, 18 bp, Betacoronavirus Severe acute respiratory syndrome coronavirus 2
  misc_feature (1)..(1): biotinylated
gcgcgacattccgaagaa

SEQ ID NO: 5 - DNA, 19 bp, Homo sapiens
agatttggacctgcgagcg

SEQ ID NO: 6 - DNA, 20 bp, Homo sapiens
  misc_feature (1)..(1): biotinylated
gagcggctgtctccacaagt

SEQ ID NO: 7 - DNA, 21 bp, Betacoronavirus Severe acute respiratory syndrome coronavirus 2
  misc_feature (1)..(1): amino linker
accccgcattacgtttggacc

SEQ ID NO: 8 - DNA, 23 bp, Betacoronavirus Severe acute respiratory syndrome coronavirus 2
  misc_feature (1)..(1): amino linker
acaatttgcccccagcgcttcag

SEQ ID NO: 9 - DNA, 23 bp, Homo sapiens
  misc_feature (1)..(1): amino linker
ttctgacctgaaggctctgcgcg